(19) United States
(12) Patent Application Publication
(10) Pub. No.: US 2012/0142623 A1
(43) Pub. Date: Jun. 7, 2012

(54) Compositions And Methods For Predicting Inhibitors Of Protein Targets

(76) Inventors: Michael Lagunoff, Seattle, WA (US); Wesley C. Van Voorhis, Seattle, WA (US); Ekachai Jenwitheesuk, Ratchaburi (TH); Ram Samudrala, Mukilteo, WA (US)

(21) Appl. No.: 12/309,143

(22) PCT Filed: Jul. 6, 2007
(86) PCT No.: PCT/US2007/072985

§ 371 Date: Sep. 14, 2009

Related U.S. Application Data
(60) Provisional application No. 60/819,426, filed on Jul. 7, 2006.

(51) Int. Cl. A61K 31/4439; A61K 31/497; A61K 31/404; A61K 31/426; A61K 31/415; G06G 7/60; A61K 31/603; A61P 31/22; A61P 31/18; A61P 33/02; C40B 30/02; A61K 31/704; A61K 31/702

(52) U.S. Cl. 514/34; 514/333; 514/252.14; 514/414; 514/370; 514/407; 514/61; 514/160; 506/8; 703/11

(57) ABSTRACT

Compositions and methods are provided for predicting inhibitors of protein targets related to treatment of infectious disease, for example, bacterial, viral, or parasitic diseases. Methods are provided for predicting inhibitors of protein targets related to treatment infectious disease, for example, microbial disease, utilizing a docking with dynamics protocol to identify inhibitors, or utilizing a protein structure energy function to identify peptide or peptidomimetic inhibitors.

### CROSS REFERENCE TO RELATED APPLICATION

This application claims the benefit of U.S. Provisional Application No. 60/819,426, filed Jul. 7, 2006, which is hereby incorporated by reference in its entirety.

### STATEMENT OF GOVERNMENT SUPPORT

This invention was made by government support by Grant No. GM 068152 from National Institutes of Health, Grant Nos. DBI-0217241 and IIS 0448502 from the National Science Foundation. The Government has certain rights in this invention.

### FIELD

The present invention relates generally to methods for predicting inhibitors of protein targets related to treatment of disease, for example, infectious disease, bacterial, viral, or parasitic diseases, or neoplastic disease. The invention further relates to methods for treatment of disease and to methods for predicting inhibitors of protein targets related to treatment of disease, for example, infectious disease, bacterial, viral, or parasitic diseases, or neoplastic disease, utilizing a docking with dynamics protocol to identify inhibitors, or utilizing a protein structure energy function to identify peptide or peptidomimetic inhibitors.

### BACKGROUND

Current therapeutic strategies for several diseases, such as Human Immunodeficiency Virus Type 1 (HIV-1) infection, have evolved from initial single target therapies to multitarget ones [1]. Single antiretroviral drug regimens against HIV-1 are no longer recommended for clinical use due to the rapid emergence of drug resistant strains after initiation of therapy [2, 3]. A combination of antiretroviral drugs targeting different viral proteins is more effective at suppressing viral growth [4]. However, for many patients, these regimens are expensive, result in greater toxicity, and poor patient adherence [5-7]. New paradigms in multitarget drug discovery have emerged [8-11], particularly for the treatment of HIV-1 infection [12, 13]. An example of a new multitarget antiretroviral drug is Cosalane, developed to inhibit multiple HIV-1 proteins (gp120, integrase, protease, and reverse transcriptase) simultaneously [14-19].

Computational screening of small molecule chemical compounds against protein targets implicated in disease has been widely used to discover lead inhibitors against diseases of interest. This process typically involves “docking” chemical compounds into the active site of the three dimensional (3D) structure of a protein target by computer simulation to identify putative leads based on calculated binding affinities of the compounds to the target. Since the number of high resolution protein structures and computer processing capabilities have increased exponentially in recent years, computational screening methods have complemented experimental high throughput screening (HTS) methods to improve the efficiency and efficacy of discovering lead inhibitors.

Malaria is one of the deadliest tropical diseases, causing more than 300 million infections yearly. Successful clearance of the malarial parasites, Plasmodium sp, from a patient's body by antimalarial drugs is impeded by the emergence of drug resistant strains. Drugs that effectively eliminate Plasmodium require short exposure durations, which reduce risk of treatment failure and emergence of drug resistant strains. Baird, N Engl J Med 352: 1565-1577, 2005.

Antimalarial drugs currently target single Plasmodium proteins. Effective therapeutic regimens require a combination of drugs that have different mechanisms of action during the same stage of the parasite's life cycle. Baird, N Engl J Med 352: 1565-1577, 2005. However, malaria is a disease that occurs mostly in tropical and subtropical areas where patients have limited access to drugs, and combination drug regimens may not succeed due to poor adherence. Fungladda, et al., Bull World Health Organ 76 Suppl 1: 59-66, 1998. New antimalarial therapies that include multi-target drugs, which are currently being used extensively to treat both infectious and inherited diseases, may have higher efficacy than single target drugs and provide a simpler regimen for antimalarial therapy. Csermely et al., Trends Pharmacol Sci 26:178-182, 2005; Ravi Chandra et al., Protein Eng Des Sel 17: 175-182, 2004.

Drug resistance is a major concern in patients treated for HIV infection and a major reason for treatment failure. There is no evidence that people infected with HIV can be cured by the currently available therapies. In fact, individuals who are treated for up to three years and are repeatedly found to have no virus in their blood experience a prompt rebound increase in the number of viral particles when therapy is discontinued. This resistance then limits the options for future treatment. A major reason that resistance develops is the patient's failure to correctly follow the prescribed treatment, such as not taking the medications at the correct time. In addition, the likelihood of suppressing the virus to undetectable levels is not as good for patients with lower CD4 cell counts and higher viral loads. Finally, if virus remains detectable on any given regimen, resistance eventually will develop.

Glycoprotein 41 (gp41) is a crucial molecule in the human immunodeficiency virus type 1 (HIV-1) envelope and is a drug target for treatment of HIV disease. Gp41 consists of four major parts: a N-terminal hydrophobic fusion peptide, a cysteine loop, and heptad repeats 1 & 2 (HR1 & 2). Enfuvirtide is the first approved peptide-based HIV-1 fusion inhibitor. It corresponds to amino acid residues 127-162 of HIV-1 gp41 (part of the HR2 domain) or residues 643-678 in the gp160 precursor of the HIV envelope glycoprotein. The inhibitor competes with the viral HR2 in binding to the HR1 trimeric coiled-coil hydrophobic groove, thereby blocking viral HR1/HR2 association. Wild et al., Proc Natl Acad Sci USA 89: 10537-10541, 1992; Jiang et al., Nature 365: 113, 1993; Wild et al., AIDS Res Hum Retroviruses 9: 1051-1053, 1993; Wild et al., Proc Natl Acad Sci USA 91: 9770-9774, 1994. Mutations of HR1 residues at the hydrophobic groove (G36, V38, Q40, N42, N43 and L45) have been reported to cause enfuvirtide resistance. Roman et al., J Acquir Immune Defic Syndr 33: 134-139, 2003; Marcelin et al., AIDS 18: 1340-1342, 2004; Wei et al., Antimicrob Agents Chemother 46: 1896-1905, 2002. Although HIV-1 strains with these HR1 mutations can escape from enfuvirtide, these strains are significantly less fit than the wild-type. Wei et al., Antimicrob Agents Chemother 46: 1896-1905, 2002; Lu et al., J Virol 78: 4628-4637, 2004; Menzo et al., Antimicrob Agents Chemother 48: 3253-3259, 2004. It is unclear how viral HR1 and HR2 mutations reduce the effectiveness of the enfuvirtide and whether these mutations subsequently restore viral fitness.

A need exists in the art for more effective treatments for microbial infectious disease, e.g., bacterial, viral, or parasitic disease, such as malaria, HIV, and herpesvirus infection. Single drug treatment against multiple targets within a disease-causing organism would improve compliance with drug treatment protocols, decrease the appearance of drug resistant strains, and decrease morbidity and mortality as a result of the bacterial, viral, or parasitic disease.

### SUMMARY

The invention provides a method for predicting inhibitors of one or more protein targets for treatment of disease, such as infectious disease or neoplastic disease, utilizing a docking with dynamics protocol to identify multi-target inhibitors useful in the treatment of infectious disease. The disease states can be, for example, infectious disease, bacterial, viral, or parasitic diseases, or neoplastic disease. The invention further provides a method for identifying candidate peptide inhibitors or candidate peptidomimetic inhibitors utilizing a peptide-based inhibitor discovery method using protein structure energy function analysis. The invention further provides a method for predicting inhibitors of one or more protein targets for treatment of one or more diseases, by calculating a binding affinity, for example, using a docking with dynamics protocol, for one or more compounds against one or more protein targets, and predicting high-ranking binding compounds as inhibitors of the one or more protein target for treatment of the one or more diseases. Pharmaceutical compositions are provided for the treatment of a broad spectrum of disease states, for example, infectious disease, bacterial, viral, or parasitic diseases, or neoplastic disease, wherein the therapeutic applications for these compositions have not been previously identified.

A method for predicting inhibitors of one or more protein targets for treatment of one or more diseases is provided which comprises providing a set of experimentally-synthesized or naturally-occurring compounds, clustering the compounds by structural similarity, calculating a binding affinity for one or more compounds representing each structurally similar cluster against one or more protein targets, ranking each representative compound by inhibitory concentration based upon calculation of binding affinity against each of the one or more protein targets for the disease or the disease-causing organism, selecting one or more high-ranking clusters of compounds, ranking compounds within the one or more high-ranking clusters based upon calculation of binding affinity against each of the one or more protein targets for the disease, and predicting high-ranking compounds as inhibitors of one or more protein target for treatment of the one or more diseases. The set of experimentally-synthesized or naturally-occurring compounds can be compounds that have been screened for one or more of toxicity, absorption, distribution, metabolism excretion, or pharmacokinetics. The set of experimentally-synthesized or naturally-occurring compounds that have been approved by the U.S. Food and Drug Administration. In a further aspect, the method comprises calculating the binding affinity using a docking with dynamics protocol. In a further aspect, the method comprises comprising ranking each compound by inhibitory concentration against two or more protein targets for treatment of the disease. In a further aspect, the method comprises ranking each compound by inhibitory concentration against the protein target for treatment of two or more diseases. In a further aspect, the method comprises predicting the inhibitor for treatment of disease using the lowest inhibitory concentration to calculate the highest binding affinity. In a further aspect, the method comprises reducing the screening time to predict inhibitors of one or more protein target for treatment of disease. The disease includes, but is not limited to, bacterial disease, viral disease, parasitic disease, or neoplastic disease.

A method for predicting inhibitors of two or more protein targets for treatment of one or more disease in a mammalian subject is provided which comprises providing a set of experimentally-synthesized or naturally-occurring drug or drug-like compounds, calculating a binding affinity using a docking with dynamics protocol for one or more compounds against two or more protein targets, ranking compounds by inhibitory concentration based upon calculation of binding affinity against each of the two or more protein targets for the disease or a disease-causing organism, and predicting high-ranking compounds as inhibitors of two or more protein target for treatment of the one or more diseases. The set of experimentally-synthesized or naturally-occurring compounds can be compounds that have been screened for one or more of toxicity, absorption, distribution, metabolism excretion, or pharmacokinetics. The method can further comprise predicting the inhibitor for treatment of disease using the lowest inhibitory concentration to calculate the highest binding affinity. The set of experimentally-synthesized or naturally-occurring compounds that have been approved by the U.S. Food and Drug Administration. The disease includes, but is not limited to, bacterial disease, viral disease, parasitic disease, or neoplastic disease.

A computer readable medium bearing computer executable instructions is provided for carrying out the method for predicting inhibitors of one or more protein targets for treatment of one or more diseases. A modulated data signal carrying computer executable instructions for performing the method is provided. At least one computing device comprising means for performing the method is provided.

A method for treating herpesvirus infection in a mammalian subject is provided which comprises administering to the mammalian subject a pharmaceutical composition in an amount effective to reduce or eliminate infection by two or more classes or species of herpesvirus or to prevent its occurrence or recurrence in the mammalian subject. The class of herpesvirus includes, but is not limited to, α-herpesvirus, β-herpesvirus, or γ-herpesvirus. In a further aspect, the species of herpesvirus is herpes simplex virus, cytomegalovirus, Kaposi's sarcoma virus, varicella zoster virus, or Epstein Barr virus.

In an embodiment, the composition is an inhibitor of a herpesvirus protease. In a detailed aspect, the composition comprises meso-5,10,15,20-Tetrakis-(N-methyl-4-pyridyl)porphine tetratosylate (TMPyP4).

A method for treating Plasmodium falciparum infection in a mammalian subject is provided which comprises administering to the mammalian subject a pharmaceutical composition capable of inhibiting two or more Plasmodium falciparum target proteins, in an amount effective to reduce or eliminate the Plasmodium falciparum infection or to prevent its occurrence or recurrence in the mammalian subject. In one aspect, the pharmaceutical composition includes, but is not limited to, KN62 (ID 274), u-74389g 2321), daunorubicin (ID 1989), nitrotetrazolium bt (ID 2174), STI-571/Imatinib (ID 637), or TMPyP4 (ID 2303). In a further aspect, the pharmaceutical composition is a telomerase inhibitor including, but is not limited to, v (ID 2288), bisindolylmaleimide iii (ID 546), methylgene_{—}05 (ID 463), remiszewski_{—}013 (ID 449), remiszewski_{—}010 (ID 448), phthalylsulfathiazole (ID 1576), or sulfaphenazole (ID 916).

A method for treating human immunodeficiency virus infection in a mammalian subject is provided which comprises administering to the mammalian subject a pharmaceutical composition comprising an inhibitor of HIV integrase in an amount effective to reduce or eliminate infection by human immunodeficiency virus or to prevent its occurrence or recurrence in the mammalian subject. The HIV integrase inhibitor includes, but is not limited to, TMPyP4, calmidazolium chloride, paromomycin, aurintricarboxylic acid, ro 31-8220 (548), dichlorobenzamil (36), catenulin (1198), kanamycin (670), or capreomycin (893).

A method for treating microbial infection in a mammalian subject is provided which comprises administering to the mammalian subject a pharmaceutical composition comprising meso-5,10,15,20-Tetrakis-(N-methyl-4-pyridyl)porphine tetratosylate (TMPyP4) in an amount effective to reduce or eliminate the microbial infection or to prevent its occurrence or recurrence in the mammalian subject. The method treats microbial infection including, but not limited to, a viral infection, bacterial infection, or parasitic infection. In a further aspect, the method treats microbial infection including, but not limited to, herpesvirus, human immunodeficiency virus, or Plasmodium falciparum.

A method for identifying a candidate peptide inhibitor or candidate peptidomimetic inhibitor of a protein target for treatment of disease is provided which comprises performing a stability analysis using a protein structure energy function to identify highly stable, partially surface-exposed elements of the protein target, designing peptide inhibitors or peptidomimetic inhibitors having the same amino acid sequence as the highly stable elements or having amino acid sequences that interacts with the highly stable element, designing derivative inhibitors by computationally mutating side chains of the peptide inhibitors or peptidomimetic inhibitors and evaluating the protein structure energy of the derivative inhibitors, and identifying the derivative inhibitor with a lower protein structure energy as the candidate peptide inhibitor of the protein target or the candidate peptidomimetic inhibitor of the protein target for treatment of disease. In one aspect, the method further comprises identifying derivative inhibitors as candidate peptide inhibitors or candidate peptidomimetic inhibitors of two or more highly stable elements in one protein target. In a further aspect, the candidate peptide inhibitors or candidate peptidomimetic inhibitors target one or more diseases. In another aspect, the method further comprises identifying the candidate peptide inhibitor or the candidate peptidomimetic inhibitor of homologous highly stable elements in two or more protein targets. In a further aspect, the candidate peptide inhibitor or the candidate peptidomimetic inhibitor target one or more diseases. In a detailed aspect, the highly stable element is a secondary structure element, a tertiary structure element, or a quaternary structure element. In a further aspect, the disease is bacterial disease, viral disease, parasitic disease, or neoplastic disease. A computer readable medium bearing computer executable instructions is provided for carrying out the method for identifying a candidate peptide inhibitor or candidate peptidomimetic inhibitor of a protein target for treatment of disease. A modulated data signal carrying computer executable instructions for performing the method is provided. At least one computing device comprising means for performing the method is provided.

A method for predicting inhibitors of two or more protein targets for treatment of one or more diseases is provided which comprises providing a set of experimentally-synthesized or naturally-occurring compounds, calculating a binding affinity for each compound against a multiplicity of protein targets, and ranking each compound by inhibitory concentration based upon calculation of binding affinity against each of the one or more protein targets for treatment of disease. In one aspect, the set of experimentally-synthesized or naturally-occurring compounds are FDA approved compounds. In a further aspect, the method comprises calculating the binding affinity using a docking with dynamics protocol. In a further aspect, the method comprises ranking each compound by inhibitory concentration against two or more protein targets for treatment of disease. In a further aspect, the method comprises ranking each compound by inhibitory concentration against the protein target for treatment of two or more diseases. In a further aspect, the method comprises predicting the inhibitor for treatment of disease by calculating the highest binding affinity. In a detailed aspect, the disease includes, but is not limited to, bacterial disease, viral disease, parasitic disease, or neoplastic disease. A computer readable medium bearing computer executable instructions is provided for carrying out the method for predicting inhibitors of two or more protein targets for treatment of one or more diseases. A modulated data signal carrying computer executable instructions for performing the method is provided. At least one computing device comprising means for performing the method is provided.

### BRIEF DESCRIPTION OF THE DRAWINGS

FIG. 6 shows a method for discovery of therapeutic drugs utilizing a multi-target and multi-disease approach.

FIG. 7 shows a method for discovery of therapeutic drugs utilizing a multi-target and multi-disease with clustering approach.

FIG. 8 shows binding patterns of 4 approved and 16 experimental multitarget drugs to 13 Plasmodium falciparum proteins.

FIG. 9 shows the HIV-1 gp41 structure used in this study is a six-helical bundle hairpin complex consisting of three chains (A, B and C).

FIG. 10 shows the list of enfuvirtide-resistant HR1 mutants and the corresponding HR2 residues.

FIGS. 11A, 11B, 11C, 11D, 11E and 11F show the surface structures of the hydrophobic groove formed by the HR1 domains of chain A and chain C.

FIG. 12 shows three dimensional molecular modeling of the inhibitor, TMPyP4, bound to herpesvirus protease.

### DETAILED DESCRIPTION

The invention provides a method for predicting inhibitors of one or more protein targets for treatment of disease, for example, infectious disease, bacterial, viral, or parasitic diseases, or neoplastic disease, utilizing a docking with dynamics protocol to identify multi-target inhibitors useful in the treatment of infectious disease. The invention further provides a method for identifying candidate peptide inhibitors or candidate peptidomimetic inhibitors utilizing a peptide-based inhibitor discovery method using protein structure energy function analysis. The candidate small chemical molecule inhibitors, peptide inhibitors, or peptidomimetic inhibitors can be further tested by in vitro cell assay or in an animal model and further determined to be effective to inhibit virus, bacteria, or parasite replication, and useful to reduce or eliminate infectious disease or to prevent its occurrence or recurrence in the vertebrate or mammalian subject. The candidate small chemical molecule inhibitors, peptide inhibitors, or peptidomimetic inhibitors can be further tested by in vitro cell assay or in an animal model and further determined to be effective against replication of neoplastic cells, and useful to reduce or eliminate neoplastic disease or to prevent its occurrence or recurrence in the vertebrate or mammalian subject.

Studies have shown that the success rates of HTS are increased by several fold when compounds were prefiltered by computational screening [20-22]. Here, we present a new computational paradigm for inhibitor discovery that is based on a combination of three factors: (1) Incorporation of dynamics of protein side chains and main chain during the docking process to more accurately evaluate binding affinities. (2) Selection of single inhibitors that bind to multiple protein targets simultaneously. (3) Using a screening library consisting of drug and drug-like compounds.

We compare the efficacy of lead compound identification by our multitarget computational screening approach to the traditional experimental HTS and single target screening approaches (FIG. 1), using HIV-1 and associated opportunistic pathogen infections, and the malarial parasite Plasmodium falciparum infection, as examples. We argue that our multitarget approach is likely to result in higher success rates, as well as reduce costs and time expended, in identifying new lead inhibitors. Our proposed approach may also be used to help minimize side effects and toxicity, thereby reducing risk in the drug discovery pipeline and increasing the likelihood of developing successful therapies against diseases of interest.

FIG. 1 shows the advantages of using a novel broad spectrum multitarget inhibitor discovery protocol against key pathogens and diseases are contrasted against traditional approaches. The major differences, corresponding to reasons why our protocol, is more effective are: (1) The use of a docking with molecular dynamics algorithm to completely take both protein and inhibitor flexibility into account. This algorithm is effective since all molecules in biology undergo dynamic/thermal motion. Traditional rigid-docking approaches do not account for this phenomenon, resulting in poor accuracy of predicting binding energies or inhibitory constants compared to our approach that takes both protein and inhibitor flexibility into account <http://compbio.washington.edu/papers/therapeutics.html>. (2) The use of compounds that bind to multiple targets simultaneously. The most effective drugs in humans (including aspirin, Gleevec) inevitably interact and bind to multiple proteins that traditional models of focusing on single target drugs fail to take into account, leading to serious side effects even after final clinical trials. The multitarget approach is a necessary one since every drug has to be effective at its site of action (for example, HIV-1 protease inhibitors have to bind and inhibit the protease molecule) and has to be effectively metabolized by body (for example, the Cytochrome P450 (CYP450) enzymes which consist of dozens of alleles). Computational screening for multitarget binding and inhibition is effective since it exploits the evolutionary fact that protein structure is vastly more conserved than nature. (3) The use of FDA approved and experimentally-synthesized drug and drug-like compounds in the computational screening process. Screening known drugs developed for other conditions against infectious diseases is likely to lead to less side effects since the toxicity, absorption, distribution, metabolism, and excretion (ADME), pharmacokinetics (PK), is typically well established in human and animal models. To our knowledge, this is the first time that these three elements have been combined to create an effective inhibitor and drug discovery protocol with predictions that have been experimentally verified to yield highly promising lead inhibitors for further drug development. The computational aspects of our protocol are fully automated and completely parallelizable and requires only a fixed initial investment in the number of CPUs purchased (i.e., the greater the number of CPUs, the more targets and compounds that can be screened; we currently use a farm of 400 CPUs which enables screening of 3200 compounds for one target in 24 hours). Our novel protocol is extremely effective and increases success rates downstream in preclinical and clinical use with a significant reduction in time, effort, and cost expended.

Computational Multitarget Screening for Diseases Caused by Multiple Microorganisms: HIV-1 and Opportunistic Pathogen Infections

Traditionally, treatment of complex diseases involving several microorganisms, especially those with a high mutation rate, requires the use multiple drugs in the therapeutic regimen, where each drug inhibits a single target in a particular microorganism. Multiple drug regimens have successfully been used in several studies to treat complex diseases and to control emergence of drug resistant strains of infectious agents [1, 4]. However, since several drugs are used in treatment regimens, this typically causes serious adverse effects and is associated with low patient adherence due to toxicity and high costs [5-7].

HIV-1, first discovered in 1981, is a pandemic human pathogen that has resulted in more than 25 million deaths caused by the Acquired Immune Deficiency Syndrome (AIDS) where the immune system ceases to function, leading to life threatening opportunistic infections. HIV-1 infected patients need to take a regimen consisting of drugs to treat both HIV-1 and opportunistic infections that arise due to immunosuppression. These patients thus present a therapeutic challenge where multitarget computational screening can provide an effective solution, since a therapeutic regimen consisting of a single drug that could simultaneously inhibit targets from multiple microorganisms would be ideal for the treatment and control of complex infectious disease combinations present these patients.

There are several HIV-related opportunistic pathogens (Table 1) that are inhibited using prophylactics [23]. Cotrimoxazole is a broad spectrum antibiotic that is effective at preventing a number of opportunistic infections. This drug is both cheap and widely available [24]. However, cotrimoxazole does not inhibit HIV-1 replication. Since HIV-1 infection is a chronic disease that requires life long antiretroviral treatment, a new generation of antiretroviral drugs simultaneously control HIV-1 and opportunistic pathogens would benefit HIV-1 patients, especially those with limited access to antiretroviral and prophylactic drugs.

TABLE 1
Partial list of opportunistic infections that occur in HIV-1 infected patients.

Bacterial infections
Mycobacterium avium complex
Salmonellosis
Syphilis and Neurosyphilis
Turberculosis
Bacillary angiomatosis
Fungal infections
Aspergillosis
Candidiasis
Coccidioidomycosis
Cryptococcal meningitis
Histoplasmosis
Pneumocystis pneumonia
Protozoal infections
Cryptosporidiosis
Isosporiasis
Microsporidiosis
Toxoplasmosis
Viral infections
Cytomegalovirus
Hepatitis
Genital herpes
Shingles
Kaposi's Sarcoma
Human papiloma virus infection
Molluscum Contagiosum
Oral hairy leukoplakia

Several drugs approved for treatment of human diseases other than HIV-1 infection have been shown to inhibit HIV-1 proteins (Table 2). These include drugs Alzheimer's disease, cancer, and infectious diseases caused by bacteria, fungi, protozoa, and viruses including HIV-1. The multitargeting features of these drugs against HIV-1 and its opportunistic pathogens were largely identified by HTS through serendipity. However, computational multitarget screening using the x-ray diffraction structures of HIV-1 protein targets from the Protein Data Bank (PDB; http://www.pdb.org) would have helped enable rational identification of these multitarget drugs.

TABLE 2
Drugs approved for treatment of infectious diseases that show inhibitory activity against HIV-1.

| | HIV-1 | | | | |
|---|---|---|---|---|---|
| Drug | Target | Inhibitory effect (uM) | Reference | Other microorganisms | Reference |
| Amphotericin B | gp41 | IC50 > 10 | [42] | Aspergillus fumigatus | [43] |
| | | | | Candida albicans | [43] |
| | | | | Candida krusei | [44] |
| | | | | Candida parapsilosis | [44] |
| | | | | Candida tropicalis | [45] |
| | | | | Cryptococcus neoformans | [43] |
| | | | | Fusarium species | [46] |
| | | | | Hepatitis B virus | [47] |
| | | | | Histoplasma capsulatum | [48] |
| Chloroquine | Integrase | IC50 = 5.14 | [49] | Cryptococcus neoformans | [50] |
| | Reverse trancriptase | IC50 > 300 | [51] | Mycobacterium tuberculosis | [52] |
| | Tat | IC50 < 50 | [53] | Plasmodium berghei | [54] |
| | | | | Plasmodium falciparum | [55] |
| Curcumin | Integrase | IC50 = 30 | [56] | Anti-Alzheimer | [57] |
| | Reverse transcriptase | NA | [58] | Anti-cancer | [59] |
| | Tat | IC50 < 30 | [53] | Anti-inflammation | [60] |
| Cyclosporin A | gag | IC50 < 1 | [61] | Candida albicans | [62] |
| | | | | Cryptococcus neoformans | [63] |
| | | | | Cryptosporidium parvum | [64] |
| | | | | Hepatitis C virus | [65] |
| | | | | Toxoplasma gondii | [66] |
| | | | | Vaccinia virus | [67] |
| Durhamycin A | Tat | IC50 = 0.0048 | [68] | Aspergillus fumigatus | [69] |
| | | | | Candida albicans | [69] |
| | | | | Cryptococcus neoformans | [69] |
| | | | | Histoplasma capsulatum | [69] |
| Enviroxime | Unknown | EC50 > 36.5 | [70] | Coxsackie virus | [71] |
| | | | | Human rhinovirus | [72] |
| | | | | Polio virus | [73] |
| Fumagillin | Vpr | EC50 = 0.015 | [74] | Encephalitozoon cuniculi | [75] |
| | Unknown | EC > 0.2 | [70] | Encephalitozoon intestinalis | [76] |
| | | | | Enterocytozoon bieneusi | [77] |
| | | | | Plasmodium falciparum | [78] |
| | | | | Vittaforma corneae | [76] |
| Hydroxychloroquine | Integrase | IC50 > 100 | [49] | Plasmodium falciparum | [79] |
| | Reverse transcriptase | NA | [80] | | |
| KNI-764 | Protease | IC50 > 0.05 | [81] | Plasmodium falciparum | [82] |
| Minocycline | Reverse transcriptase | IC50 = 1200 | [83] | Cryptosporidium parvum | [84] |
| | Unknown | EC50 < 22 | [85] | Enterococcus faecalis | [86] |
| | | | | Enterococcus faecium | [86] |
| | | | | Mycobacterium fortuitum | [87] |
| | | | | Mycobacterium tuberculosis | [88] |
| | | | | Mycoplasma pneumoniae | [89] |
| | | | | Staphylococcus aureus | [86] |
| | | | | Staphylococcus pyogenes | [86] |
| | | | | Streptococcus pneumoniae | [86] |
| | | | | Toxoplasma gondii | [90] |
| Suramin | gp120 | ED50 = 7.7 | [91] | Cytomegalovirus | [92] |
| | Integrase | IC50 = 2.4 | [93] | Herpes simplex virus | [94] |
| | Reverse transcriptase | IC50 = 1.4 | [95] | Influenza A virus | [96] |
| | | | | Rhinovirus | [70] |
| | | | | Sandfly fever virus | [97] |

The data presented in Table 2 provides evidence for single drugs (or a combination of 2-3 drugs) that can inhibit infection by multiple bacteria, fungi, protozoa, and viruses, including HIV-1, simultaneously. A striking example is the inhibitor KNI-764/JE-2164 (row 9, Table 2) that inhibits both HIV-1 protease and the plasmepsin enzyme target from the malarial parasite Plasmodium malariae, the complexes of which have both been solved by x-ray diffraction (PDB identifiers 1msm and 2anl, respectively). Another example is minocycline (row 9, Table 2), a broad spectrum antibiotic that has been shown to possess inhibitory activity against HIV-1 in vitro. Our docking simulations predict that it inhibits HIV-1 integrase (Jenwitheesuk and Samudrala, manuscript submitted). The former example provides strong evidence for the existence and utility of multitarget drugs (since the binding mode of a single inhibitor bound to targets from two very different and destructive pathogens has been elucidated to atomic detail). The latter illustrates how computational screening methods can be used to identify targets and binding modes of multitarget inhibitors discovered fortuitously.

Table 2 focuses on drugs for which there is published evidence supporting their simultaneous effectiveness against HIV-1 and associated opportunistic pathogen infections. Below, we illustrate how our computational multitarget screening approach can be used to discover effective inhibitors against the malarial parasite P. falciparum.

Computational Multitarget Screening for Diseases Caused by a Single Microorganism: Plasmodium falciparum

Previous studies using structure-based single target computational screening of two different large compound libraries against two cysteine proteases (falcipain-2 and falcipain-3) of the malarial parasite P. falciparum have shown low success rates [25, 26]. A computational screen using 355,000 compounds from the Available Chemical Directory (ACD) database predicted 100 putative inhibitors, of which only seven demonstrated experimental inhibitory activity in vitro [25]. A second experiment on the same targets using 241,000 compounds from the ChemBridge database predicted 100 putative inhibitors, of which eleven demonstrated experimental inhibitory activity in vitro [26]. The results of these single target computational screening studies not only indicated a low success rate of approximately 10% at identifying P. falciparum inhibitors, but also that this approach was not able to identify potential targets of a given compound since some of the predicted compounds inhibited P. falciparum growth but failed to inhibit the expected targets.

Two recent experimental HTS studies yielded an even lower success rate of approximately 3% for P. falciparum growth inhibition: Chong et al. screened 2,687 drug and drug-like compounds for P. falciparum growth inhibition and found 87 antimalarial compounds with activity ≦10 μM [27]. Weisman et al. similarly screened 2,160 compounds and found 72 antimalarial compounds with >70% growth inhibition relative to control at 1 μM [28].

We previously screened a library of 2,344 drug and drug-like compounds against fourteen P. falciparum proteins [29] (FIG. 2) using a computational docking with dynamics protocol that predicts inhibitors of target protein structures by simultaneously considering protein-inhibitor flexibility and dynamics [30, 31]. The screened compounds were ranked according to the consensus weighted rank (the average of the ranks of the compound observed in all simulations divided by the number of proteins predicted to be inhibited by that compound; the lower the rank, the better the predicted efficacy), which is a measure of the multitargeting capability of a compound. Sixteen of the top ranking compounds based on their predicted multitargeting capability were experimentally evaluated for P. falciparum growth inhibition, and five compounds predicted to have no inhibitory activity were used as a negative control.

FIG. 2 shows nineteen compounds with antimalarial activity were selected from multitarget computational screening study (top seven rows) [29] and the HTS studies performed by Chong et al. (middle) [27] and Weisman et al. (bottom twelve rows) [28]. Shown for each compound are their predicted inhibitory constants against each of fourteen P. falciparum proteins (shaded boxes; dark brown indicates highest inhibition) and the total number of proteins predicted to be inhibited. Some proteins have inhibitors in the mid-picomolar range (for example, Dihydrofolate reductase) but others have predicted inhibitors that are in the micromolar range (for example, 1-Cys peridoxin). Our predictions indicate that a compound such as U-74389G is more likely to inhibit Glutathione reductase and Lactate dehydrogenase (all picomolar inhibitory constants) than 1-Cys peridoxin, Dihydrofolate reductase, Glutathione-s-transferase, Protein kinase-5, S-Adenosyl-L-homocysteine hydrolase, and Thymidylate synthase (micromolar to nanomolar inhibitory constants). We experimentally evaluated sixteen of our top ranking compounds based on their predicted multitargeting capability to inhibit P. falciparum growth in cell culture. In addition, we compared the multitarget computational screening predictions to two experimental HTS studies evaluating more than 2,000 compounds to discover inhibitors of P. falciparum growth [27,28]. Many of the compounds experimentally demonstrated to inhibit P. falciparum growth in cell culture are predicted to inhibit multiple proteins. By experimentally screening only sixteen predictions from a computational library of 2,344 compounds, six sub-micromolar antimalarial lead candidates were obtained at a fraction of the time, effort, and cost that would have been required to perform experimental HTS. Overall, the success rate of approximately 38% of multitarget computational screening is significantly higher than the rates of approximately 3% produced by the two experimental HTS studies for identifying antimalarial inhibitors.

Experimental verification was performed by adding compounds in serial dilutions to the chloroquine-sensitive strain 3D7 and the chloroquine-resistant strain K1 of P. falciparum cultures. The mean ED50 was determined from at least two or more measurements for each compound. Six of sixteen top predictions had ED50s (≦1 μM against either the 3D7 or K1 strains, and all five negative control compounds did not inhibit 3D7 P. falciparum growth. The overall prediction accuracy was 52% ( 11/21), with a success rate of 38% ( 6/16) at identifying promising lead candidate compounds against chloroquine-sensitive and chloroquine-resistant strains of P. falciparum (Table 3). The success rate (38%) of our multitarget screening approach is a significant improvement over the previous single target computational screening (10%) [25, 26] and HTS (3%) rates [27, 28].

TABLE 3
Analysis and comparison of antimalarial compounds predicted by our multitarget
approach and those obtained by experimental high throughput screening.

| | Computational | | |
|---|---|---|---|
| | prediction | | Experimental verification |
| | No. of | Consensus | ED50 in 3D7 |
| Compound | targets | weighted rank | (μM) |

A. Computational multitarget

| | | | | |
|---|---|---|---|---|
| | | | | ED50 in K1 |
| | | | | (μM) |
| U-74389G | 8 | 4.67 | 0.83 | <1 |
| Daunorubicin | 4 | 4.70 | 0.13 | <1 |
| Bisindolylmaleimide X | 8 | 4.92 | 1-10 | 1-10 |
| Nitrotetrazolium | 7 | 5.43 | 0.50 | <1 |
| KN62 | 7 | 5.47 | 0.69 | <1 |
| Sulfasalazine | 7 | 5.93 | >40 | >40 |
| TMPyP4 | 6 | 5.99 | 1 | 1 |
| STI-571 | 10 | 6.23 | 6 | 7.50 |
| GW8510 | 11 | 6.44 | 1-10 | 1 |
| PIPER | 10 | 7.04 | 10-40 | 10-40 |
| Succinylsulfathiazole | 4 | 8.05 | >40 | >40 |
| Bisindolylmaleimide II | 10 | 9.02 | 1-10 | 1-10 |
| Protoporphrin | 5 | 9.18 | 10-40 | 10-40 |
| Coelenterazine | 11 | 10.42 | 10-40 | 1-10 |
| Bisindolylmaleimide VII | 11 | 12.54 | 1-10 | 1-10 |
| R-(+)-WIN55212-2 | 7 | 14.61 | 10-40 | 10-40 |
| SU5402 (negative control) | 1 | 20.00 | >40 | Not tested |
| SU6656 (negative control) | 7 | 26.56 | >40 | Not tested |
| SU4984 (negative control) | 2 | 44.00 | >40 | Not tested |
| Roscovitine (negative control) | 0 | NA | 10 | Not tested |
| SU5614 (negative control) | 0 | NA | 10 | Not tested |

B. High throughput screen
[27, 28]

| | | | | |
|---|---|---|---|---|
| | | | | % Inhibition at |
| | | | | 10 μM in |
| Daunorubicin | 4 | 4.70 | Not tested | 99.6 |
| Epirubicin | 5 | 5.96 | Not tested | 98.4 |
| Metergoline | 4 | 11.22 | 5.40 | 85.9 |
| Topotecan | 4 | 12.42 | Not tested | 80.1 |
| Aminopterin | 6 | 12.76 | 1.60 | 95.7 |
| Risperidone | 5 | 14.17 | 9.90 | 67.5 |
| Dihydroergotamine [27] | 4 | 14.79 | 4.00 | 88.1 |
| Dihydroergotamine [28] | 4 | 14.79 | 3.00 | Not tested |
| Methotrexate | 6 | 17.11 | 0.05 | 98.4 |
| Vindesine | 2 | 20.17 | Not tested | 81.9 |
| Azlocillin | 2 | 21.50 | 1.50 | Not tested |
| Raloxifene | 4 | 24.58 | <0.50 | 97.1 |
| Puromycin | 3 | 26.67 | Not tested | 98.9 |
| Astemizole | 4 | 28.83 | 0.23 | 97.6 |

Table 3 footnote. For each compound, the number of proteins predicted to be inhibited, a consensus weighted rank (the average of the ranks of the compound observed in all our simulations divided by the number of proteins predicted to be inhibited by that compound; the lower the rank, the better the predicted efficacy), which is a measure of the multitargeting capability of a compound, and the experimental result for P. falciparum growth inhibition are given. (A) Experimental verification of multitarget compounds predicted to inhibit P. falciparum growth. Of the 21 compounds tested, sixteen compounds were predicted to have high inhibition based on their multitargeting capability, and five compounds were used as a negative control. All computational predictions were repeated in triplicate with randomized starting positions for the simulations. Experimental verification was performed by adding compounds in serial dilutions to the chloroquine-sensitive strain 3D7 and the chloroquine-resistant strain K1 of P. falciparum cultures. The mean effective dose where 50% of P. falciparum growth was inhibited (ED50) was determined from at least two or more measurements for each compound. Six of sixteen top predictions had ED50s ≦1 μM against either the 3D7 or K1 strains, and all five negative control compounds did not inhibit 3D7 P. falciparum growth. Our overall prediction accuracy is 52% ( 11/21), with a success rate of 38% ( 6/16) at identifying promising lead inhibitors against chloroquine-sensitive and chloroquine-resistant strains of P. falciparum. (B) Experimental verification of compounds predicted to inhibit P. falciparum growth based on the experimental high throughput screening studies. Chong et al. screened 2,687 compounds for P. falciparum growth inhibition and found 87 antimalarial compounds with activity ≦10 μM [27]. Weisman et al. tested 2,160 compounds and found 72 antimalarial compounds with >70% growth inhibition relative to control at 1 μM [28]. The thirteen unique compounds listed from the two sets (dihydroergotamine is repeated) were selected from 73 and 15 overlapping compounds that we had screened computationally for which experimental data were provided. These compounds would have been predicted by us to inhibit P. falciparum growth based on their multitargeting capability (i.e., low consensus weighted rank). All the other compounds that overlapped with the HTS libraries have high consensus weighted ranks, and we hypothesize that any inhibitory activities of these other compounds result from other mechanisms and not by inhibition of the fourteen proteins screened by us. All the thirteen compounds listed are considered antimalarial inhibitors using the criteria of the HTS studies. Using our more stringent criteria of ED50 of ≦1 μM or 95% inhibition at 10 μM would have resulted in a success rate of 54% ( 7/13). The success rates of the multitarget computational screening of 38% (A) and 54% (B) are thus significantly higher than the HTS success rates (3%), and are achieved by screening only sixteen compounds experimentally, at a fraction of the time, effort, and cost that would have been required to perform experimental HTS.

Multitarget computational screening may also be applied to predict potential targets of a given inhibitor identified by HTS. This is illustrated in FIG. 2 which shows the predicted targets of thirteen unique overlapping compounds between our computational library and the experimental libraries of the two HTS studies [27, 28], which would have been predicted to inhibit P. falciparum growth based on their multitargeting capability (i.e., low consensus weighted rank) and for which experimental inhibition values were provided. Some targets have inhibitors in the mid-picomolar range (for example, Dihydrofolate reductase) but others have predicted inhibitors that are in the micromolar range (for example, 1-Cys peridoxin). Our predictions indicate that a compound such as U-74389G is more likely to inhibit Glutathione reductase and Lactate dehydrogenase (all picomolar inhibitory constants) than 1-Cys peridoxin, Dihydrofolate reductase, Glutathione-s-transferase, Protein kinase-5, S-Adenosyl-L-homocysteine hydrolase, and Thymidylate synthase (micromolar to nanomolar inhibitory constants). This application of multitarget computational screening is therefore useful in prioritizing targets for further study of compounds with unknown inhibitory mechanisms.

Toxicity Minimization

Although a multitarget inhibitor is expected to bind to multiple disease protein targets with high affinity, it may undesirably inhibit other human proteins, leading to toxicity. Strategies to identify and predict side effects such as acute toxicity, mutagenicity, and carcinogenicity have been extensively studied and reviewed [32-38].

In terms of computational screening, a library of approved drug and drug-like compounds being evaluated in clinical trials or those with known toxicity profiles may be used to identify initial lead inhibitors, thereby reducing the likelihood of deleterious side effects. Additional compounds may be selected from larger libraries containing synthetic and natural compounds, where the entire library is filtered and categorized into groups according to their onset and severity of toxicity. This can be accomplished by using data in the TOXNET database (http://toxnet.nlm.nih.gov) [39] or examining their Absorption Distribution Metabolism Elimination Toxicity (ADME-Tox) profiles [40]. Focusing on infectious disease targets that are not similar to essential proteins in humans also reduces the likelihood of a toxic reaction.

Toxicity filtering may also be done by structural similarity comparison or SMILES strings similarity search [41] between successful lead candidates and compounds with known toxicity profiles. The purpose of categorizing compounds is to prioritize the experimental verification of the computational screening results for a given set of targets or diseases. Compounds with moderate toxicity may be included in our screening library for diseases that require short courses of treatment. On the other hand, compounds with a moderate degree of toxicity may be eliminated from our library for chronic diseases.

Potential side effects may also be predicted using computational multitarget screening lead inhibitors against essential human proteins with known structure. Lead inhibitors can also be screened against proteins involved in human drug metabolism (such as the Cytochrome P450 family of enzymes) to ensure their proper metabolism and minimize the risk of producing toxic metabolites.

Efficacy and Efficiency of Multitarget Computational Screening

Multitarget computational screening using a docking with dynamics protocol and drug-like compound library has the promise to significantly enhance the identification of lead inhibitors for drug development. This protocol identifies inhibitors that simultaneously and selectively bind to multiple targets with high affinity, in contrast to most drug development strategies that focus only on single target inhibition. The efficacy and efficiency of multitarget computational screening has the potential to significantly reduce time, effort, and cost to obtain promising lead candidates for drug development.

The present study provides evidence that multitarget inhibitors exist for complex diseases involving several microorganisms such as HIV-1 and associated opportunistic pathogen infections, and that these lead compounds are excellent starting points for further chemical modification to improve potency and specificity against targets of interest. We also demonstrate that computationally predicted multitarget antimalarial inhibitors show high potency at inhibiting P. falciparum growth in vitro, with a higher success rate than single-target computational screening and experimental HTS. Onset of drug resistance, a significant problem with both HIV-1 and P. falciparum infection, may be significantly delayed by inhibiting multiple targets simultaneously.

An important application of multitarget computational screening is that it may be used to identify potential targets for a drug whose inhibitory mechanism is unknown. Since we start with drug and drug-like compounds that are well characterized in terms of their pharmacological properties, the probability of success as a drug further down the development pipeline is increased. Modification of lead chemical compounds using medicinal chemistry rules can be performed in silico. Side effect screening against essential human proteins can also be performed computationally to refine these candidates, and screening against important human enzymes involved in eliminating drugs from the body may help ensure proper metabolism with nontoxic metabolite buildup. The opinion and evidence presented here is largely in the context of infectious disease targets. However, our computational multitarget approach can be readily extended to other complex human diseases such as cancer, which require inhibition of multiple proteins in developmental pathways to be effective.

Developing a comprehensive computational pipeline that integrates the concepts presented here will not only lead to the discovery of new inhibitors but also has the potential to enable significant advances in the efficacy and efficiency of the entire process of drug discovery and development, from in vitro and in vivo preclinial studies to clinical trials.

The present methods have identified small molecule compounds, small chemical molecule inhibitors, peptide inhibitors, or peptidomimetic inhibitors useful as broad spectrum antimicrobial treatments. For example, the compound TMPyP4 has been identified by methods of the present invention as a candidate inhibitor of herpesvirus replication, and as a candidate therapeutic composition for treatment of a broad spectrum of herpesvirus infectious disease. This compound has been shown by molecular modeling studies to bind to herpesvirus protease and by in vitro studies to inhibit infection and replication of several classes of herpesvirus, e.g., α-herpesvirus, β-herpesvirus, or γ-herpesvirus. The compound TMPyP4 is also a candidate inhibitor of HIV-1 integrase and a therapeutic composition for treatment of human HIV-1 infection. The compound TMPyP4 is a candidate multitarget inhibitor of Plasmodium falciparum proteins and a therapeutic for treatment of Plasmodium falciparum infection.

A method for identifying a candidate peptide inhibitor or candidate peptidomimetic inhibitor of a protein target for treatment of disease is provided which comprises performing a stability analysis using a protein structure energy function to identify highly stable, partially surface-exposed elements of the protein target, designing peptide inhibitors or peptidomimetic inhibitors having the same amino acid sequence as the highly stable elements or having amino acid sequences that interacts with the highly stable element, designing derivative inhibitors by computationally mutating side chains of the peptide inhibitors or peptidomimetic inhibitors and evaluating the protein structure energy of the derivative inhibitors, and identifying the derivative inhibitor with a lower protein structure energy as the candidate peptide inhibitor of the protein target or the candidate peptidomimetic inhibitor of the protein target for treatment of disease.

A method for predicting inhibitors of two or more protein targets for treatment of one or more diseases is provided which comprises providing a set of experimentally-synthesized or naturally-occurring compounds, calculating a binding affinity for each compound against a multiplicity of protein targets, and ranking each compound by inhibitory concentration based upon calculation of binding affinity against each of the one or more protein targets for treatment of disease.

A method for predicting inhibitors of one or more protein targets for treatment of one or more diseases is provided which comprises providing a set of experimentally-synthesized or naturally-occurring compounds, clustering the compounds by structural similarity, calculating a binding affinity for one or more compounds representing each structurally similar cluster against one or more protein targets, ranking each representative compound by inhibitory concentration based upon calculation of binding affinity against each of the one or more protein targets for the disease or the disease-causing organism, selecting one or more high-ranking clusters of compounds, ranking compounds within the one or more high-ranking clusters based upon calculation of binding affinity against each of the one or more protein targets for the disease, and predicting high-ranking compounds as inhibitors of one or more protein target for treatment of the one or more diseases.

“Docking with dynamics” is a computational protocol that predicts the binding mode (configuration) and energy of a small molecule (chemical compound) to a protein structure. This is essentially a combination of two techniques: molecular docking (implemented by the software AutoDock; http://www.scripps.edu/mb/olson/doc/autodock/; Morris, et al., J. Computational Chemistry, 19: 1639-1662, 1998.) and molecular dynamics (implemented by the software NAMD; http://www.ks.uiuc.edu/Research/namd/; Phillips, et al., Journal of Computational Chemistry, 26: 1781-1802, 2005).

Docking with dynamics or docking with dynamics and clustering has been used to predict susceptibility of infectious viruses to drug treatment. Docking with dynamics has been used to predict HIV protease mutant drug binding affinities and to predict HIV protease drug resistance/susceptibility. Jenwitheesuk E, and Samudrala R., Bioorg Med Chem Structural Biology, 3: 2-10, 2003. Jenwitheesuk E, and Samudrala R., Antiviral Therapy 10: 157-166, 2005. Docking with dynamics has been used to predict inhibitors against the SARS coronavirus proteinase. Jenwitheesuk E, and Samudrala R., Bioorg Med Chem Lett. 13: 3989-3992, 2003.

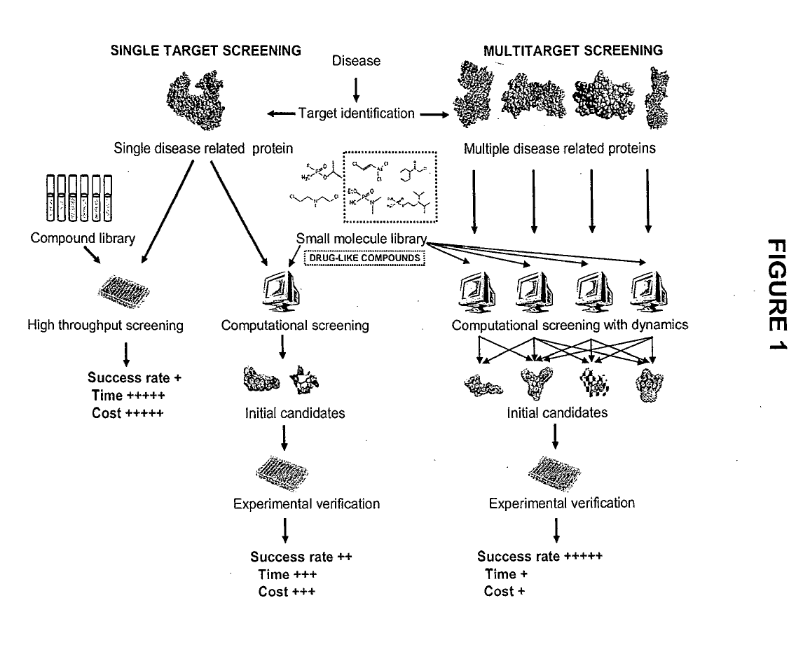

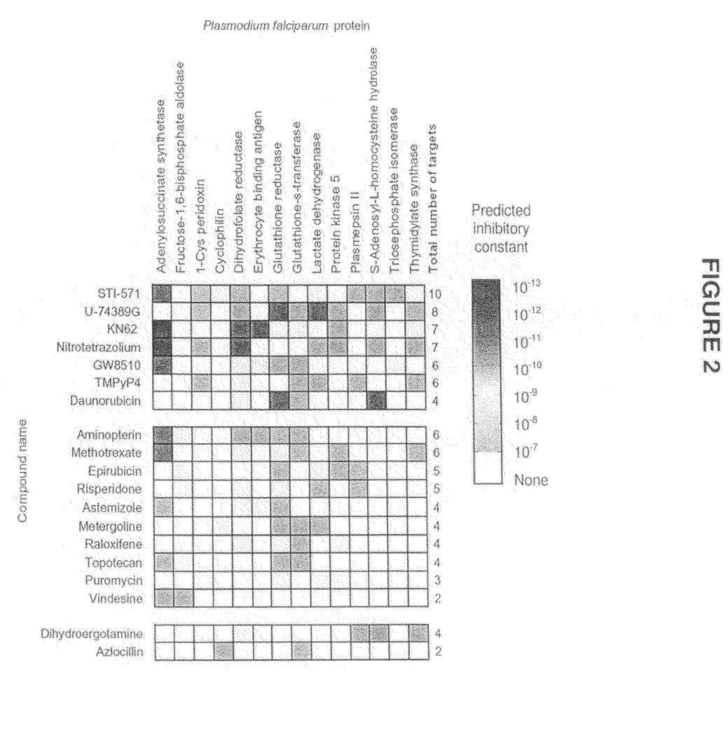

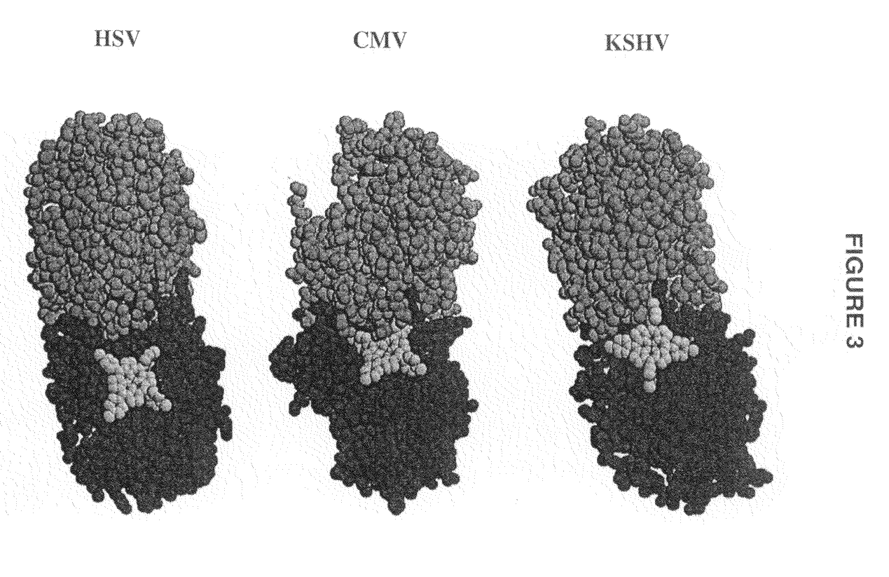

Multi-Target Inhibitor Discovery Using Docking with Dynamics Protocol
Docking with dynamics protocol has been used to predict effectiveness of HIV protease inhibitors against CMV protease. See FIG. 6.
Docking with dynamics protocol has been used as a general protocol to predict inhibitors (from a pool of FDA experimental and approved drugs) against single targets in different herpesviruses.
The clustering of a large database of drugs based on conformational similarity using SMILES strings has been used to aid multi-target inhibitor discovery. See FIG. 7.
Docking with dynamics protocol and application has been used to identify potential multitarget antimalarial drugs from a pool of FDA experimental and approved drugs. See FIG. 8 and Table 4.
The use of docking with dynamics can be used to screen for side effects.

### Peptide-Based Inhibitor Discovery Using Protein Structure Energy Functions

Peptide based inhibitor discovery using protein structure energy functions has been used to identify mutations in the heptad repeat 2 (HR2) region of HIV-1 glycoprotein 41 (gp41) that enhance the stability of enfuvirtide-resistant HIV-1 gp41 hairpin structure.
The use of an all-atom scoring function for protein structure prediction (RAPDF) has been developed to identify derivatives of the HIV gp41 peptidomimetic fusion inhibitor.
RAPDF can be used to identify hyperstable regions in a protein or protein complex (on the surface, or in the interacting partner) as potential peptidomimetic inhibitors, and using RAPDF to design variants as potential peptide-based inhibitors.
The RAPDF methodology can be extended to multiple targets using the same peptides.

### Combination Therapies

Using the above methodology, identify small-molecule inhibitors and peptide-based multi-target inhibitors against infectious disease and inherited disease.

### Application of Method to Predict Inhibitors

Broad spectrum small molecule inhibitors of herpesvirus proteases from α-, β-, and γ-herpesviruses, e.g., HSV-1, HSV-2, VZV, EBV, CMV, or KSHV.
   Best predictions showed inhibitory activity against all three classes of herpesviruses (α, β, and γ) in cell culture. The viruses tested were KSHV, HSV-1, and CMV.
   Inhibition of viral growth is comparable or better than known anti-herpes drugs in the market, e.g., acyclovir, gancylovir, foscarnet.
   Inhibitor is unique in that it inhibits all three viruses/viral classes.
   All three classes of herpesvirus cause life-threatening diseases in immunocompromised patients.
   HSV drugs alone represent greater than a $2 billion dollar yearly market and growing at a 10% rate. Nearly 90 million people worldwide are infected with the genital herpes virus, and about 25 million of them suffer frequent outbreaks of painful blisters and sores.
   Acylovir and its analogues are nucleoside analogues/inhibitors. The herpesvirus protease inhibitors identified herein are a novel type of anti-herpes agent that may be used in combination therapy with acyclovir, gancylovir, foscarnet and analogues thereof.
   The herpesvirus protease inhibitor has been evaluated in mouse models of cancer and found to very nontoxic.
   Topical applications are therefore possible with a high likelihood of success.
Further examples:
Approximately 2300 FDA-approved and experimental compounds are typically screened using the docking with dynamics protocol.
von Grotthuss, et al., “Ligand.Info Small-Molecule Meta-Database,” Comb Chem High Throughput Screen, 8: 757-761, 2004.
Peptide-based inhibitors of HIV-1 gp41 fusion. See Table 7.
Multi-target small molecule inhibitors of HIV-1 (targeting Integrase and TAR). See Table 12.
Peptide-based inhibitors influenza hemagglutinin.
Multi-target small molecule inhibitors of cancer.
Multi-target small molecule inhibitors of fourteen targets from Plasmodium falciparum. See FIG. 8 and Table 4.
Multi-target small molecule inhibitors of Trypanosoma brucei.
Multi-target small molecule inhibitors of Trypanosoma cruzi.
Multi-target small molecule inhibitors of Leishmania major.
Peptide-based inhibitors of Dengue virus envelope.
Multitarget small molecule inhibitors of HIV-1; Predicted inhibitors of HIV-1 capsid. See Table 14.
Multitarget small molecule inhibitors of Mycobacterium tuberculosis. See Table 15;

“Highly stable element” refers to a secondary, tertiary, or quaternary structural element that is highly stable as measured by techniques known in the art, for example, by RAPDF stability scores of protein structure as described herein.

“Highly stable surface exposed element” refers to highly stable elements that are exposed on the protein surface.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., a microbial infectious disease). “Treating” further refers to any indicia of success in the treatment or amelioration or prevention of the disease, condition, or disorder (e.g., a microbial infectious disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with an autoimmune disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of a microbial infectious disease but does not yet experience or exhibit symptoms, inhibiting the symptoms of a microbial infectious disease (slowing or arresting its development), providing relief from the symptoms or side-effects of microbial infectious disease (including palliative treatment), and relieving the symptoms of microbial infectious disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Experimentally-synthesized or naturally-occurring drug or drug-like compounds” refers to compounds that have been screened or tested for one or more of toxicity, absorption, distribution, metabolism excretion, or pharmacokinetics. The experimentally-synthesized or naturally-occurring drug or drug-like compounds may also be approved for use by the U.S. Food and Drug Administration.

The term “modulator” includes inhibitors and activators. Inhibitors are agents that, e.g., bind to, partially or totally block stimulation, decrease, prevent, delay activation, inactivate, desensitize, or block replication of the infectious virus, bacteria, or parasite, e.g., antagonists. Activators are agents that, e.g., bind to, stimulate, increase, open, activate, facilitate, enhance activation, sensitize a receptor or factor that will block replication of the infectious virus, bacteria, or parasite, e.g., agonists. Modulators include agents that, e.g., alter the interaction of the infectious virus, bacteria, or parasite with proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring receptor ligands, e.g., with altered activity, as well as naturally occurring and synthetic ligands, antagonists, agonists, small chemical molecules and the like. Such assays for inhibitors and activators include, e.g., applying putative modulator compounds to a cell infected with a virus, bacteria, or parasite and then determining the functional effects on virus, bacteria, or parasite replication, as described herein. Samples or assays comprising cells with infectious virus, bacteria, or parasite can be treated with a potential activator, inhibitor, or modulator are compared to control samples without the inhibitor, activator, or modulator to examine the extent of inhibition of replication by the infectious virus, bacteria, or parasite. Control samples (untreated with inhibitors) can be assigned a relative activity value of 100%. Inhibition of replication by the infectious virus, bacteria, or parasite is achieved when the activity value relative to the control is about 80%, optionally 50% or 25-0%.

“Inhibitors,” “activators,” and “modulators” of infectious microbial disease, e.g., an infectious disease caused by a herpesvirus, a human immunodeficiency virus, or Plasmodium falciparium. in cells are used to refer to inhibitory, activating, or modulating molecules, respectively, identified using in vitro and in vivo assays for compounds that block replication of the infectious virus, bacteria, or parasite.

“ED₅₀” means the dose of a drug which produces 50% of its maximum response or effect.

“Effective amount” refers to concentrations of components such as drugs or small molecule inhibitors, or compositions effective for producing an intended result including a method for treating a disease or condition in a mammalian subject, e.g., herpesvirus infection, malaria, cancer or neoplastic disease, with compounds or therapeutic compositions of the invention. An effective amount of compounds or therapeutic compositions reduces or eliminates infectious disease or cancer, or prevents it's occurrence or recurrence in the mammalian subject.

“Administering” or “administration” refers to the process by which compounds or therapeutic compositions of the invention are delivered to a patient for treatment purposes for a disease or condition in the patient, e.g., herpesvirus infection, malaria, cancer or neoplastic disease. Compounds or therapeutic compositions can be administered a number of ways including parenteral (e.g. intravenous and intraarterial as well as other appropriate parenteral routes), oral subcutaneous, inhalation, or transdermal. compounds or therapeutic compositions of the invention are administered in accordance with good medical practices taking into account the patient's clinical condition, the site and method of administration, dosage, patient age, sex, body weight, and other factors known to physicians.

“Animal” or “mammalian subject” refers to mammals, preferably mammals such as humans, primates, rats, or mice. Likewise, a “patient” or “subject” to be treated by the method of the invention can mean either a human or non-human animal, to whom treatment, including prophylactic treatment, with the compounds or therapeutic compositions of the present invention, is provided. For treatment of those conditions or disease states that are specific for a specific animal such as a human patient, the term refers to that specific animal.

Therapeutic Applications

The compounds and modulators identified by the methods of the present invention can be used in a variety of methods of treatment. Thus, the present invention provides compositions and methods for treating an infectious microbial disease, e.g., an infectious disease caused by a herpesvirus, a human immunodeficiency virus, or Plasmodium falciparium.

Exemplary infectious disease, include but are not limited to, viral, bacterial, fungal, or parasitic diseases. The polypeptide or polynucleotide of the present invention can be used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. Alternatively, the polypeptide or polynucleotide of the present invention can also directly inhibit the infectious agent, without necessarily eliciting an immune response.

Viruses are one example of an infectious agent that can cause disease or symptoms that can be treated or detected by a polynucleotide or polypeptide of the present invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but not limited to, the following Gram-Negative and Gram-positive bacterial families and fingi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a small chemical molecule inhibitor, a polypeptide inhibitor, or a peptidomimetic inhibitor of viral, bacterial, or parasite replication of the present invention could either be by administering an effective amount of the small chemical molecule inhibitor, the polypeptide inhibitor, or the peptidomimetic inhibitor to the patient, or by removing cells from the patient, supplying the cells with a polynucleotide of the present invention, and returning the engineered cells to the patient (ex vivo therapy). Moreover, the polypeptide or peptidomimetic of the present invention can be used as an antigen in a vaccine to raise an immune response against infectious disease.

Formulation and Administration of Pharmaceutical Compositions

The invention provides pharmaceutical compositions comprising small chemical molecule inhibitors, a polypeptide inhibitors, or a peptidomimetic inhibitors of the invention. As discussed above, the inhibitors of the invention can be used to inhibit expression or activity of viral, bacterial, or parasitic proteins involved in infection or replication. Such inhibition in a cell or a non-human animal can generate a screening modality for identifying compounds to treat or ameliorate a microbial infectious disease. Administration of a pharmaceutical composition of the invention to a subject is used to generate a toleragenic immunological environment in the subject. This can be used to tolerize the subject to an antigen.

The small chemical molecule inhibitor, polypeptide inhibitor, or peptidomimetic inhibitor of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of a small chemical molecule inhibitor, a polypeptide inhibitor, or a peptidomimetic inhibitor of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of small chemical molecule, polypeptide, or peptidomimetic in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Small chemical molecule inhibitor, polypeptide inhibitor, or peptidomimetic inhibitor of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

The small chemical molecule inhibitor, polypeptide inhibitor, or peptidomimetic inhibitor of the invention can also be administered in sustained delivery or sustained release mechanisms, which can deliver the formulation internally. For example, biodegradable microspheres or capsules or other biodegradable polymer configurations capable of sustained delivery of a peptide can be included in the formulations of the invention (see, e.g., Putney, Nat. Biotechnol. 16: 153-157, 1998).

For inhalation, the small chemical molecule inhibitor, polypeptide inhibitor, or peptidomimetic inhibitor of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The small chemical molecule inhibitor, polypeptide inhibitor, or peptidomimetic inhibitor of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186: 507-515, 1997.

In one aspect, the pharmaceutical formulations comprising a small chemical molecule inhibitor, a polypeptide inhibitor, or a peptidomimetic inhibitor of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble small chemical molecule inhibitors, polypeptide inhibitors, or peptidomimetic inhibitors of the invention have been attached to the surface of the monolayer or bilayer. For example, peptides can be attached to hydrazide-PEG-(distearoylphosphatidyl)ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the small chemical molecule inhibitors, polypeptide inhibitors, or peptidomimetic inhibitors of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.

The pharmaceutical compositions are generally formulated as sterile, substantially isotonic and in full compliance with all Good Manufacturing Practice (GMP) regulations of the U.S. Food and Drug Administration.

Treatment Regimens: Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of small chemical molecule inhibitors, polypeptide inhibitors, or peptidomimetic inhibitors adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., Gennaro, (ed), Remington's Pharmaceutical Sciences, 20th edition, Mack Publishing Company, pp. 1127-1144, 2000; Egleton, Peptides 18: 1431-1439, 1997; Langer, Science 249: 1527-1533, 1990.

In therapeutic applications, compositions are administered to a patient suffering from an infectious disease in an amount sufficient to at least partially arrest the condition or a disease and/or its complications. For example, in one aspect, a soluble small chemical molecule, polypeptide, or peptidomimetic pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The invention will be further described with reference to the following examples; however, it is to be understood that the invention is not limited to such examples.

### EXEMPLARY EMBODIMENTS

### Example 1

### Effectiveness of Our Drug Discovery Protocol Against Herpesviruses

The first experimental verification of our computational predictions were with TMPyP4, the top ranking inhibitor predicted to bind to all known herpesvirus proteases (see FIG. 3). This is also the inhibitor that we have characterized most computationally and experimentally, so we focus primarily on this inhibitor. There currently do not exist any known herpes protease inhibitors in clinical trials (or use). Traditional approaches to design an inhibitor against this molecule is likely to fail since it has an extremely shallow active site pocket. However, protease dimerisation is essential for protein function and our top ranking inhibitor not only fit around the active site (covering it) but also intercolated itself in the dimer interface. Our predicted mode of action was therefore two fold: covering the surface of the active site, and inhibition of dimerisation in a reversible manner (i.e., as dimers formed, our inhibitor would bind which would disrupt the dimer, resulting in the inhibitor binding weakly and likely released, which would lead to reformation of the dimer, and so on). FIG. 3 illustrates this mode of action. FIG. 4 shows the experimentally determined disassociation constants which is completely consistent with our prediction. FIG. 5 shows the effectiveness of our top ranking inhibitor against the representative members of the three major classes of herpesviruses, demonstrating that our predictions, Kd measurements, and in vitro studies are completely consistent with each other. TMPyP4 is the only known compound that is capable of inhibiting all three classes of herpesviruses.

FIG. 3 shows binding modes of our top prediction to the structures of HSV (left), CMV (middle), and KSHV (right) proteases. The protease dimer structure is shown as a space-fill view in green and blue. Our top ranking (highest affinity) inhibitor (yellow) is predicted to bind to sites around the shallow active site pocket and in the dimer interface, thereby disabling the function of the protease molecule

FIG. 4 shows disassociation constants (Kd) of our top ranking inhibitors for HSV, CMV, and KSHV experimentally determined using Surface Plasmon Resonance (SPR). Four inhibitors were evaluated; our top ranking one (TMPyP4) clearly binds to all three proteases as predicted. Our second ranking inhibitor (Bisindolylmaleimide) binds with lower affinity than our top one, but also targets all three protease molecules. A third inhibitor, GW8510, predicted to bind fails (i.e., a misprediction). A fourth inhibitor, SU6566, predicted and verified to not bind was used as a negative control. The Kd of TMPyP4 is in the micromolar range which is consistent with our prediction of the inhibitor binding to herpes protease monomers. The dimerisation constant of herpes proteases is also in the micromolar range. Our prediction of the Kd of TMPyP4 binding to the protease dimer is in the nanomolar range, but since we predict it disrupt dimerisation, this nanomolar binding is observed only transiently. Our inhibitor prediction is completely consistent with the observed experimental data which is verified further by cell culture studies in FIG. 4 and compared to existing antiherpes drugs.

FIG. 5 shows inhibition of TMPyP4, our top predicted inhibitor, against HSV, CMV, and KSHV (top three panels). Cells in the absence or presence of TMPyP4, and antiherpes drugs acylovir or gancyclovir. Virus from the infected cells were then titered. The titers of each infection from 2 or 3 separate experiments are shown. Vero cells were infected with HSV-1 strain F. HFF cells were infected with CMV. Our inhibitor works as comparably or better than existing antiherpes drugs against HSV and CMV (top left two panels). Using a different assay, TMPyP4 also inhibited KSHV, a gamma herpesvirus (top right most panel). Note that all panels except the top right most panel display the amount of virus on a log scale whereas the KSHV shows the fold reduction. Our computationally predicted broad spectrum human herpesvirus protease inhibitors is effective in vitro against members from all three classes and is comparable or better than antiherpes drugs. Our protease inhibitor acts synergistically with acylovir (a nucleoside analogue that inhibits replication) so much that it almost completely eliminates all virus when used together (bottom left panel). After passage for several cycles with both acylovir and TMPyP4, complete resistance to acylovir occurs, whereas our inhibitor still continues to be effective (bottom right panel).

### Example 2

### Identification of Drugs with High Affinity Binding to Multiple Plasmodium falciparum Proteins

Antimalarial drugs currently target single Plasmodium proteins. Effective therapeutic regimens require a combination of drugs that have different mechanisms of action during the same stage of the parasite's life cycle. Baird, N Engl J Med 352: 1565-1577, 2005. However, malaria is a disease that occurs mostly in tropical and subtropical areas where patients have limited access to drugs, and combination drug regimens may not succeed due to poor adherence. Fungladda, et al., Bull World Health Organ 76 Suppl 1: 59-66, 1998. New antimalarial therapies that include multi-target drugs, which are currently being used extensively to treat both infectious and inherited diseases, may have higher efficacy than single target drugs and provide a simpler regimen for antimalarial therapy. Csermely et al., Trends Pharmacol Sci 26:178-182, 2005; Ravi Chandra et al., Protein Eng Des Sel 17: 175-182, 2004. This study predicted a list of drugs that bind to the active site of multiple Plasmodium falciparum proteins with high affinity.

Protein-inhibitor docking with dynamics has been used as a general protocol to predict inhibitors from a pool of FDA experimental and approved drugs against multiple targets in malaria. A computational protein-inhibitor docking with dynamics protocol was used to calculate the binding affinities of 1105 approved and 1239 experimental drugs (obtained from ChemBank) against thirteen Plasmodium proteins whose structures have been determined by X-ray crystallography. <http://ligand.info/ligand_info_subset_{—}1.sdf.gz>, accessed May 1, 2005. Binding affinity calculations were carried out using AutoDock version 3.0.5 with a Lamarckian genetic algorithm. Each drug was first placed into the active site of the protein to find the most stable binding mode. The protein-drug complexes were consequently solvated in a water shell with sodium and chloride ions. One hundred steps of energy minimization were applied, followed by 0.1 picoseconds (ps) of molecular dynamics simulation to each complex using the XPLOR software version 3.851. The conformations at 0.1 ps were used for the protein-drug binding affinity calculations.

For each protein, a given drug was docked into the active site and allowed to move in an exhaustive manner to find the most stable binding conformation. The protein-drug binding affinity in terms of the inhibitory constant (Kᵢ) was calculated every time the drug molecule was moved. After repeating this procedure for all the drugs for each protein, the twenty drugs with the lowest Kᵢ values were considered high affinity drug candidates. See FIG. 8. Further details of the molecular dynamics simulation and docking protocols are given elsewhere. Jenwitheesuk et al., Antivir Ther 10:157-166, 2005; Jenwitheesuk et al., Bioorg Med Chem Lett 13: 3989-3992, 2003; Jenwitheesuk et al., AIDS 19: 529-531, 2005.

Twenty multi-target drugs that showed high affinity across two or more proteins were predicted. Four are approved drugs; KN62 (targeting three proteins), Protoporphyrin IX, Phthalylsulfathiazole, and Sulfaphenazole (targeting two proteins each), and the other sixteen are experimental, each targeting up to six proteins. The best drugs in terms of multi-target functionality were STI-571 (targeting six proteins), and Bisindolylmaleimide x, GW8510, and Piper (targeting five proteins each). The best combination of two drugs was Bisindolylmaleimide x and GW8510, which together target ten Plasmodium proteins. An analysis of five known single-target antimalarial drugs against these proteins showed that our calculated Kᵢs for these drugs match well with experimentally determined values (when available), and usually rank within the top 5^{th} percentile compared to all our drugs. See Table 4.

Vaccines attacking multiple Plasmodium proteins have been proposed with promising results. Nussenzweig et al., Science 265: 1381-1383, 1994. In a similar fashion, designing new antimalarial drugs that target multiple Plasmodium proteins simultaneously is proposed. Our computational drug screening protocol provides evidence for twenty approved and/or experimental drugs targeting thirteen Plasmodium proteins. The drug candidates listed here may be experimentally tested for inhibition of Plasmodium growth, and used as a starting point for further design of a high efficacy multi-target antimalarial drug.

TABLE 4
Comparison of the calculated Kᵢs of five known antimalarial drugs and the drugs predicted to have the highest binding [symbol]
ffinity. The calculated Kᵢ values for these drugs are similar to the experimentally determined ones
and rank within the top 5th [symbol] ercentile in comparison to all our drugs.

| | | Experimental | Calculated Kᵢ of drug |
|---|---|---|---|
| Protein (PDB identifier) | Calculated Kᵢ (rank) | inhibitory activity (M) | with highest affinity |
| [symbol] ihydrofolate reductase with pyrimethamine | 2.80 × 10⁻⁸ (102) | Kᵢ = 2.00 × 10⁻¹⁰ | 7.16 × 10⁻¹² |
| [symbol] ihydrofolate reductase (1J3I) | 1.10 × 10⁻⁸ (48) | Kᵢ = 1.10 × 10⁻¹¹ | 7.16 × 10⁻¹² |
| [symbol] asmepsin II (1LF3) | 1.58 × 10⁻⁹ (7) | Kᵢ = 1.00 × 10⁻⁷ | 1.01 × 10⁻¹⁰ |
| [symbol] Adenosyl-L-homocysteine hydrolase (1V8B) | 3.83 × 10⁻⁸ (222) | IC₅₀ = 3.10 × 10⁻⁶ | 4.88 × 10⁻¹² |
| [symbol] ymidylate synthase (1J3I) | 2.75 × 10⁻⁷ (131) | Data not available | 1.02 × 10⁻⁹ |

[symbol]

indicates data missing or illegible when filed

### Example 3

### Identification of Drugs with High Affinity Binding to Multiple Plasmodium falciparum Proteins

Table 5 shows the results for the 16 inhibitors that were tested. Four inhibitors were found to have very strong binding (e.g., KN62, U-74389G, Daunorubicin, and Nitrotetrazolium) Two inhibitors have moderate binding (e.g., Imatinib (Gleevec) and TmPyP4). Based on this study, these six drugs work against malaria.

TABLE 5

| | | | | | | | ED₅₀ |
|---|---|---|---|---|---|---|---|
| | | | | Drug | | ED₅₀ (μM) | (μM) |
| 1 | 2 | 3 | 4 | ID | Drug name | in 3D7 | in K1 |
| Y | 12 | Y | Y | 274 | KN62 | 0.690 | <1 |
| Y | 12 | Y | Y | 2321 | U-74389G | 0.825 | <1 |
| Y | 6 | Y | Y | 1989 | Daunorubicin | 0.125 | <1 |
| Y | 12 | Y | Y | 2174 | Nitrotetrazolium | 0.500 | <1 |
| Y | 13 | Y | M | 637 | Imatinib (Gleevec) | 0.6 | 7.5 |
| Y | 12 | M | M | 2303 | TMPyP4 | 1 | 1 |
| Y | 11 | N | N | 577 | GW8510 | 1-10 | 1 |
| Y | 4 | N | N | 551 | Bisindolylmaleimide VII | 1-10 | 1-10 |
| Y | 6 | N | N | 545 | Bisindolylmaleimide II | 1-10 | 1-10 |
| Y | 10 | N | N | 553 | Bisindolylmaleimide X | 1-10 | 1-10 |
| Y | 7 | Y | N | 1973 | Coelenterazine | 10-40 | 1-10 |
| Y | 4 | N | N | 17 | R-(+)-WIN55 212-2 | 10-40 | 10-40 |
| Y | 4 | Y | N | 711 | Protoporphrin | 10-40 | 10-40 |
| Y | 14 | Y | N | 2216 | Piper | 10-40 | 10-40 |
| Y | 10 | N | N | 1420 | Sulfasalazine | >40 | >40 |
| Y | 2 | N | N | 1575 | Succinylsulfathiazole | >40 | >40 |

NEGATIVE CONTROL

| | | | | | | |
|---|---|---|---|---|---|---|
| N | 0 | N | N | 281 | Roscovitine | 10 |
| N | 0 | N | N | 639 | SU4984 | N/A |
| N | 0 | N | N | 640 | SU5402 | N/A |
| N | 0 | N | N | 641 | SU5614 | N/A |
| N | 1 | N | N | 642 | SU6656 | 10 |

column 1 - predicted to work (using original filter with computational protein-inhibitor docking with dynamics protocol)
column 2 - predicted to work (consensus/rank 10 filter − higher the better)
column 3 - predicted to work (important target filter)
column 4 - experimentally determined to work (Y—yes (<=1uM ED₅₀)|N—no|M—moderate/uncertain)

### Example 4

### Mechanism of Drug Resistance and Design of Peptidomimetic Inhibitors Against Drug Resistant Strains of Human Immunodeficiency Virus

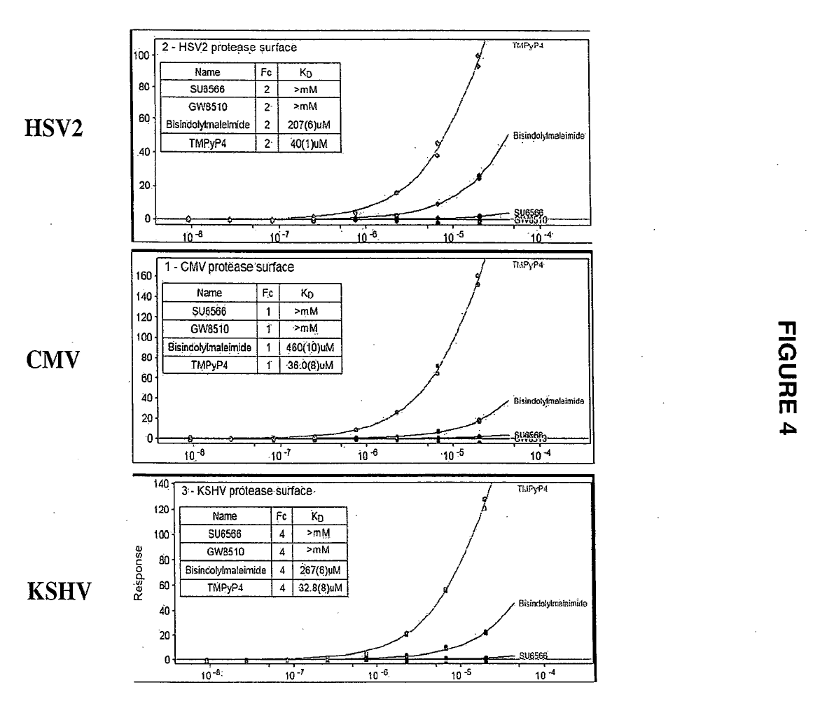

Glycoprotein 41 (gp41) is a crucial molecule in the human immunodeficiency virus type 1 (HIV-1) envelope and is a drug target for treatment of HIV disease. Gp41 consists of four major parts: a N-terminal hydrophobic fusion peptide, a cysteine loop, and heptad repeats 1 & 2 (HR1 & 2). gp41 mediates fusion of viral and target-cell membranes by inserting its fusion peptide into the target-cell membrane after formation of CD4/gp120/chemokine-receptor complex. The HR1 trimer is a three-stranded coiled-coil structure that associates with HR2 in an antiparallel orientation to form a six-helical bundle hairpin complex. Formation of the HR1/HR2 hairpin complex brings viral and target-cell membranes in close proximity to enable membrane fusion and viral entry. See FIG. 9. Wyatt et al., Science 280: 1884-1888, 1998; Chan et al., Cell 93: 681-684, 1998; Weissenhorn et al., Mol Membr Biol 16: 3-9, 1999.

FIG. 9 shows the HIV-1 gp41 structure used in this study is a six-helical bundle hairpin complex consisting of three chains (A, B and C). Each chain consists of three parts: HR1, HR2 and cysteine loop. (I) The HR1 domains in chain C (HR1-C) and chain A (HR1-A) form a coiled-coil structure that allows HR2 of chain A (HR2-A) to bind in an antiparallel orientation. Enfuvirtide, a synthetic peptide that structurally mimics HR2, inhibits viral and target cell membrane fusion by competitively binding with the HR1 and blocking HR1/HR2 association. (II) Mapping of residue-residue interactions between HR1 and HR2 was carried out by defining HR2 residues with Cα-Cα distances <7.5 Å from the following HR1 residues: G36, V38, Q40, N42, N43 and L45. The mapping diagrams show the top view of HR1-A/HR2-A/HR1-C complex. The residue number and the wild-type amino acid code of each residue are labeled in circle connected by lines that illustrate the sequence order. The shortest Cα-Cα distance between interacting residues is given.

Enfuvirtide is the first approved peptide-based HIV-1 fusion inhibitor. It corresponds to amino acid residues 127-162 of HIV-1 gp41 (part of the HR2 domain) or residues 643-678 in the gp160 precursor of the HIV envelope glycoprotein. The inhibitor competes with the viral HR2 in binding to the HR1 trimeric coiled-coil hydrophobic groove, thereby blocking viral HR1/HR2 association. Wild et al., Proc Natl Acad Sci USA 89: 10537-10541, 1992; Jiang et al., Nature 365: 113, 1993; Wild et al., AIDS Res Hum Retroviruses 9: 1051-1053, 1993; Wild et al., Proc Natl Acad Sci USA 91: 9770-9774, 1994. Mutations of HR1 residues at the hydrophobic groove (G36, V38, Q40, N42, N43 and L45) have been reported to cause enfuvirtide resistance. Roman et al., J Acquir Immune Defic Syndr 33: 134-139, 2003; Marcelin et al., AIDS 18: 1340-1342, 2004; Wei et al., Antimicrob Agents Chemother 46: 1896-1905, 2002. Although HIV-1 strains with these HR1 mutations can escape from enfuvirtide, these strains are significantly less fit than the wild-type. Wei et al., Antimicrob Agents Chemother 46: 1896-1905, 2002; Lu et al., J Virol 78: 4628-4637, 2004; Menzo et al., Antimicrob Agents Chemother 48: 3253-3259, 2004. It is unclear how viral HR1 and HR2 mutations reduce the effectiveness of the enfuvirtide and whether these mutations subsequently restore viral fitness.

In this study, a computational protein modeling approach was used to investigate the effects of amino acid changes in HR2 at positions that directly interact with the enfuvirtide-resistant HR1 residues. Such changes in HR2 were shown to improve the structural stability of the HR1/HR2 hairpin complex, thereby enhancing drug resistance level and viral fitness of the enfuvirtide-resistant strains.

Generation of mutant theoretical structures. The theoretical structure of a six-helical bundle HIV-1 gp41 hairpin complex consisting of HR1, HR2 and the cysteine loop (Protein Data Bank identifier 1IF3) was used as a template for creating mutant structures. This structure was previously modeled using NMR restraints from the simian immunodeficiency virus (SIV) gp41 ectodomain as a template. Caffrey, Biochim Biophys Acta 1536: 116-122, 2001. Wild-type side chains were substituted with the mutant side chains based on a backbone-dependent side chain rotamer library and a linear repulsive steric energy term provided by SCWRL version 3.0. Bower et al., J Mol Biol 267: 1268-1282, 1997. The resulting all-atom models were energy minimized for 200 steps using the Energy Calculation and Dynamics (ENCAD) program. Levitt et al., J Mol Biol 46: 269-279, 1969; Levitt, J Mol Biol 82: 393-420, 1974; Levitt, J Mol Biol 168: 595-620, 1983; Levitt et al., Comp Phys Comm 91: 215-231, 1995.

Prediction of the stability of the hairpin complex structures. A residue-specific all-atom probability discriminatory function (RAPDF) score was used as an indicator of the structural stability of a given hairpin complex. Samudrala et al., J Mol Biol 275: 895-916, 1997. This function has been used as a key component of protein structure prediction methods that work well in the CASP blind prediction experiments. Hung et al., Nucleic Acids Res 33: W77-80, 2005.

A residue-specific all-atom probability discriminatory function (RAPDF) score was used as a proxy for the structural stability of a given hairpin complex. The RAPDF score is calculated based on the conditional probability of a conformation being native-like given a set of inter-atomic distances. The conditional probabilities are compiled by counting frequencies of distances between pairs of atom types in a database of protein structures. The distances observed are divided into 1.0 Å bins ranging from 3.0 Å to 20.0 Å. Contacts between atom types in the 0-3 Å range are placed in a separate bin, resulting in a total of 18 distance bins. Distances within a single residue are not included in the counts. Tables of scores were compiled proportional to the negative log conditional probability that one is observing a native conformation given an interatomic distance for all possible pairs of the 167 atom types for the 18 distance ranges from a database of known structures. Given a set of distances in a conformation, the probability that the conformation represents a correct fold is evaluated by summing the scores for all distances and the corresponding atom pairs. A complete description of this formalism has been published elsewhere. Samudrala et al., J Mol Biol 275: 895-916, 1997.

Comparison of the RAPDF stability scores with experimentally-determined inching temperatures. A set of ten gp41 mutant structures were generated for which the melting temperatures (Tm) are available. Sanders et al., J Virol 76: 8875-8889, 2002; Markosyan et al., Virology 10: 302:174-184, 2002. The RAPDF scores for these structures and the wild-type structure was calculated and compared to the melting temperatures. See Table 6. The goal was to determine how well the predicted RAPDF scores correlate with experimentally-determined gp41 stability.

TABLE 6
Correlation of the RAPDF scores and the corresponding melting
temperatures (Tm) for 10 HR1 or HR2 single mutants, as well as
the wild-type, from two sources [21-22]. The correlation
coefficient is 0.82 (0.86 when the single outlier (I62P) is removed)
showing that as the RAPDF score increases (i.e., indicating lower
stability), the melting temperature decreases.

| Mutation | Melting temperature (C.°) | RAPDF score | Source |
|---|---|---|---|
| Wild-type | 76 | −35.12 | [21] |
| I48G | 46 | −33.14 | [21] |
| I48P | 34 | −33.26 | [21] |
| L55V | 72 | −34.40 | [21] |
| T58P | 44 | −34.26 | [21] |
| Wild-type | 78 | −35.12 | [22] |
| I62A | 55 | −34.14 | [22] |
| I62P | 40 | −34.03 | [22] |
| I62S | 51 | −34.21 | [22] |
| I62V | 71 | −34.46 | [22] |
| I131A | 71 | −34.38 | [22] |
| I131S | 67 | −34.47 | [22] |

Comparison of the RAPDF stability scores with the EC₅₀ values and the viral fitness levels. A set of seven HR1-mutant/HR2-wild-type structures were generated for which the EC₅₀ values (the molar concentrations of enfuvirtide that inhibits viral-target cell membrane fusion by 50%) and the viral fitness levels are available. See Table 7. Greenberg et al., J Antimicrob Chemother 54: 333-340, 2004; Lu et al., J Virol 78: 4628-4637, 2004. The second set of these mutants was duplicated from the first set with additional HR2 compensatory mutations (S138Y and Q139R). The RAPDF scores were calculated for the structures in both sets and compared the scores with the experimental enfuvirtide EC₅₀ values and the viral fitness levels. The goal was to determine how well the RAPDF scores (and by inference, protein stability) predict EC₅₀ and viral fitness.

TABLE 7
Correlation of the RAPDF scores, the enfuvirtide EC₅₀ values,
and the viral fitness levels.

| | | | RAPDF score | |
|---|---|---|---|---|
| | Viral fitness | EC₅₀ | HR2 | With HR2 |
| HR1 mutation | level | (mg/L) | wild-type | mutation |
| Wild-type | +++++ | 0.012 | −35.12 | — |
| N42T | ++++ | 0.045 | −34.66 | −35.13 (Q139R) |
| V38A | +++ | 0.188 | −34.98 | −35.36 (Q139R) |
| N42T + N43K | ++ | 0.388 | −34.69 | −36.21 (S138Y, |
| | | | | Q139R) |
| N42T + N43S | ++ | 0.727 | −34.65 | −35.66 (S138Y, |
| | | | | Q139R) |
| V38A + N42D | + | 1.685 | −34.89 | −35.42 (Q139R) |
| V38A + N42T | + | 1.782 | −34.49 | −35.13 (Q139R) |
| V38E + N42S | data not | 6.156 | −33.73 | −35.02 (Q139R) |
| | available | | | |

The RAPDF scores of the HR1 mutant structures range from −34.98 to −33.78, which are higher than that of the wild-type (−35.12). The scores directly correlate with the previously published EC₅₀ values and inversely correlate with the viral fitness levels (represented by the + symbol). The correlation coefficient between the RAPDF scores and the EC₅₀ values is 0.9. After amino acid substitutions at positions 138 and 139 in HR2, the RAPDF scores of the HR1 mutant structures range from −36.21 to −35.02 indicating that compensatory HR2 mutations improve the structural stability of the HR1/HR2 hairpin complexes.

Mapping of HR1/HR2 residue-residue interactions. The HR1/HR2 residue-residue interactions were mapped by finding the corresponding HR2 residues that had Cα-Cα distances within 7.5 Å from the following enfuvirtide-resistant HR1 residues: G36, I37, V38, Q40, N42, N43, L44 and L45. See FIG. 9. Roman et al., J Acquir Immune Defic Syndr 33: 134-139, 2003; Marcelin et al., AIDS 18: 1340-1342, 2004; Wei et al., Antimicrob Agents Chemother 46: 1896-1905, 2002.

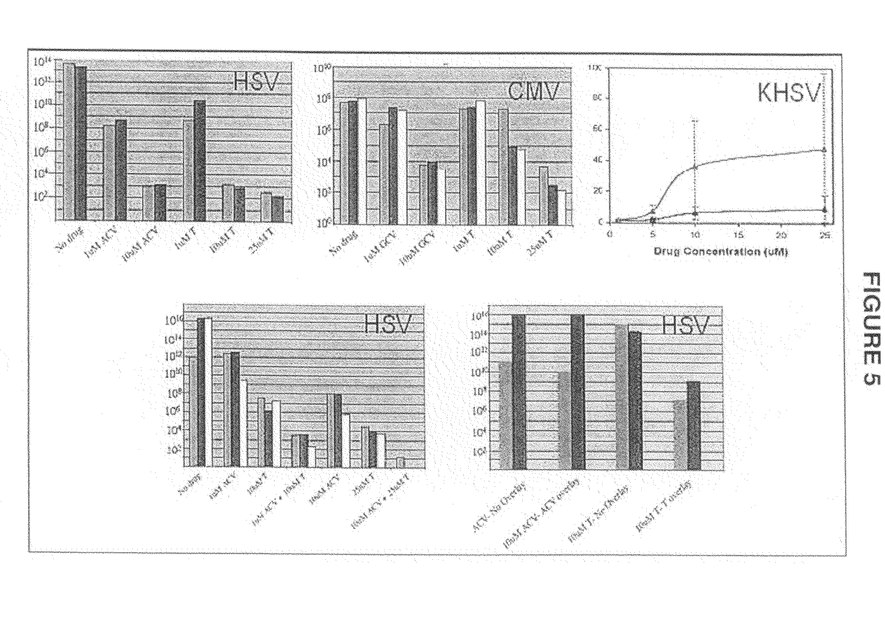

Generation of enfuvirtide-resistant HR1/HR2 hairpin structures. An initial set of 28 mutant structures of the HR1/HR2 hairpin complex were generated such that each structure consisted of one enfuvirtide-resistant mutation on HR1 and a wild-type amino acid at the corresponding HR2 residue. This initial set was used to generate nineteen other sets of HR1/HR2 double mutants such that the corresponding HR2 wild-type residue was changed to each of the remaining nineteen amino acids. At the end of this step, a total of 560 structures of the enfuvirtide-resistant HR1/HR2 mutant complex were obtained. The mutation patterns of the generated hairpin structures are shown in FIG. 10.

FIG. 10 shows the list of enfuvirtide-resistant HR1 mutants and the corresponding HR2 residues. The amino acid codes in each bar are the compensatory amino acids at the corresponding HR2 positions predicted to improve structural stability of the hairpin complex. The height of the amino acid code represents the RAPDF score of the HR1/HR2 hairpin complex. The lower the RAPDF score the higher the structural stability of the hairpin complex.

Identification of the amino acids at the corresponding HR2 positions that improve structural stability of the hairpin complex. The RAPDF scores of 560 HR1/HR2 mutant structures calculated from the previous step were compiled in a 28×20 table. Each row of this table contains 20 RAPDF scores calculated from 20 hairpin structures. Each of these 20 hairpin structures consisted of one enfuvirtide-resistant mutation on HR1 and one of the 20 amino acids at the corresponding HR2 residue. The amino acid at the corresponding HR2 residue was identified that improved structural stability of the hairpin complex by calculating the mean and standard deviation of the RAPDF scores on each row. The cutoff was set at one standard deviation under the mean. The hairpin structure that had the RAPDF score lower than the cutoff was defined as having improved structural stability. The amino acid at the corresponding HR2 residue of this structure was defined as a “compensatory amino acid” that improved the structural stability of the hairpin complex. See FIG. 10.

Designing enfuvirtide derivatives against enfuvirtide-resistant strains. From the mapping of residue-residue interaction and the identification of HR2 compensatory amino acid studies, six corresponding HR2 residues (134, 138, 139, 141, 142 and 145) were identified that were in close contact with the enfuvirtide-resistant HR1 residues. The HR2 compensatory amino acids identified based on the RAPDF scores were: D, H, (L), N, Q, S, Y for residue 134; H, N, Q, (S), T, W, Y for residue 138; K, N, (Q), R for residue 139; H, K, R, (Q) for residue 141; H, K, M, N, (Q), R, Y for residue 142 and F, (N), R, W, Y for residue 145. (Wild-type amino acids are indicated by parenthesis.)

An initial set of eighteen HR1 mutant structures reported to cause enfuvirtide resistance in patients were generated. Roman et al., J Acquir Immune Defic Syndr 33: 134-139, 2003; Marcelin et al., AIDS 18: 1340-1342, 2004; Wei et al., Antimicrob Agents Chemother 46: 1896-1905, 2002. The list of the HR1 mutations is shown in Table 7. For each HR1 mutant structure, mutations were introduced at residues 134, 138, 139, 141, 142 and 145 of the HR2. The wild-type amino acids of these six HR2 residues were randomly replaced by the compensatory amino acids. This yielded a total of 27,440 HR1/HR2 mutant structures, each of which had different HR2 mutation patterns. The same procedure was applied to all eighteen HR1 mutant structures so that a total of 493,920 HR1/HR2 mutant structures were obtained in this step.

All structures were constructed as previously described scored using the RAPDF function. The RAPDF scores were categorized into eighteen groups according to eighteen HR1 mutation patterns. The scores were ranked in ascending order to find the general patterns of HR2 mutations that could stabilize hairpin complexes of all enfuvirtide-resistant mutants. See Table 8.

TABLE 8
List of enfuvirtide-resistant mutations and amino acid
substitutions at six HR2 residues that improve structural
stability of the HR1/HR2 hairpin complexes.

| | HR2 mutation (residues | | | | | | |
|---|---|---|---|---|---|---|---|
| HR1 mutation | 134, 138, 139, 141, 142, 145) | | | | | | RAPDF score |
| Wild-type | Wild-type | | | | | | −35.12 |
| Wild-type | H | Y | R | R | R | Y | −37.92 |
| Wild-type | — | — | — | — | — | F | −37.01 |
| Wild-type | — | — | — | — | — | R | −37.28 |
| G36D | — | — | — | — | — | F | −37.85 |
| G36S | — | — | — | — | — | F | −38.01 |
| V38A | — | — | — | — | — | F | −37.97 |
| V38A + N42D | — | — | — | — | — | F | −38.44 |
| V38A + N42T | — | — | — | — | — | F | −37.81 |
| V38E + N42S | — | — | — | — | — | R | −36.71 |
| V38E | — | N | — | — | — | R | −37.16 |
| V38M | — | — | — | — | — | — | −37.86 |
| Q40H | — | — | — | — | — | — | −37.88 |
| N42D | — | — | — | — | — | — | −38.50 |
| N42E | — | — | — | — | — | — | −38.69 |
| N42S | — | — | — | — | — | — | −37.75 |
| N42T | — | — | — | — | — | — | −37.76 |
| N42T + N43S | — | — | — | — | — | — | −37.55 |
| N43D | — | — | — | — | — | — | −38.11 |
| N43K | — | — | K | — | — | — | −37.94 |
| N43S | — | — | — | — | — | — | −37.65 |
| L45M | — | — | — | — | — | — | −37.92 |

Enfuvirtide derivative designed according to these HR2 mutation patterns may have high structural stability against both wild-type and enfuvirtide-resistant strains. The amino acid code in a column that is identical to the first sequence is represented by the (—) symbol.

### Example 5

### Comparison of the RAPDF Stability Scores with Experimentally-Determined Melting Temperatures

Table 6 shows the RAPDF stability scores and the corresponding melting temperatures for 10 HR1 or HR2 single mutants, as well as the wild-type, from two sources. Sanders et al., J Virol 76: 8875-8889, 2002; Markosyan et al., Virology 10: 302:174-184, 2002. The correlation coefficient is 0.82 (0.86 when the single outlier (I62P) is removed) showing that as the RAPDF score increases (i.e., indicating lower stability), the melting temperature decreases. The best score is obtained for the wild-type (which also has the highest melting temperature reported in both sources). This result indicates that the RAPDF score, a key component of protein structure prediction methods that work well, may be used as a predictor of structural stability. Hung et al., Nucleic Acids Res 33: W77-80, 2005.

### Example 6

### Comparison of the RAPDF Stability Scores with the EC₅₀ Values and the Viral Fitness Levels

The RAPDF scores of seven HR1 mutants were compared with the EC₅₀ values of enfuvirtide and the viral fitness levels. Table 7 shows that the wild-type structure had the best RAPDF score (−35.12). The scores increased to range from −34.98 to −33.78 for all seven HR1 mutant structures and were directly correlated with the EC₅₀ values and inversely correlated with the viral fitness levels. The correlation coefficient between the RAPDF scores and the EC₅₀ values was 0.9. See Table 7. This result indicates that the structural stability scores may be used to accurately estimate the viral fitness levels and the inhibitory activity of the enfuvirtide and its derivatives.

### Example 7

### Mapping of HR1/HR2 Residue-Residue Interactions

Structural arrangement of eight HR1 residues (G36, I37, V38, Q40, N42, N43, L44 and L45) that have been associated with enfuvirtide resistance were considered. Roman et al., J Acquir Immune Defic Syndr 33: 134-139, 2003; Marcelin et al., AIDS 18: 1340-1342, 2004; Wei et al., Antimicrob Agents Chemother 46: 1896-1905, 2002. FIG. 9 illustrates that all eight residues are in the hydrophobic groove of the HR1, where I37 and L44 form the bottom of the groove and G36, V38, Q40, N42, N43 and L45 form the binding surface for the HR2 domain. Mapping of HR1/HR2 residue-residue interaction revealed six corresponding HR2 residues (L134, S138, Q139, Q141, Q142 and N145) that interact with the binding surface of the HR1 with Cα-Cα distances <7.5 Å. The side chains of these corresponding HR2 residues are in close contact with the eight HR1 residues except those between L45 and I135. Their side chains do not interact with each other, though the Cβ-Cα distance was 6.77 Å. The side chain of L45 is in between S138 and Q139, whereas the side chain of I135 points toward Q52. See FIG. 9.

### Example 8

### Identification of the Amino Acids at the Corresponding HR2 Positions that Improve Structural Stability of the Hairpin Complex

The RAPDF scores of the enfuvirtide-resistant HR1 mutant structures (G36D/S, V38A/E/M, Q40H, N42D/E/S/T, N43D/K/S and L45M) were calculated and compared to the wild-type structure score. The RAPDF scores for these mutant structures ranged from −35.06 to −34.09, which were higher than the score of the wild-type structure (−35.12). This indicates that the enfuvirtide-resistant mutants have lower hairpin structural stability than the wild-type.

The residue-residue mapping revealed six corresponding HR2 residues that interact with eight enfuvirtide-resistant HR1 residues. It was then hypothesized that the compensatory amino acid substitutions at these corresponding HR2 residues may improve structural stability of the HR1/HR2 hairpin complex. To identify these compensatory amino acids, the wild-type amino acid was replaced at the corresponding HR2 positions with each of the other nineteen amino acids.

The RAPDF scores of these HR1/HR2 mutant structures indicated that the HR2 compensatory amino acids were: D, H, (L), N, Q, S, Y for residue 134; H, N, Q, (S), T, W, Y for residue 138; K, N, (Q), R for residue 139; H, K, R, (Q) for residue 141; H, K, M, N, (Q), R, Y for residue 142 and F, (N), R, W, Y for residue 145. (Wild-type amino acids are indicated by parenthesis.) The RAPDF scores of the HR1 mutants possessing one of these HR2 compensatory amino acids ranged from −36.01 to −34.11 indicating enhancement of the structural stability of the hairpin complex after introducing the compensatory amino acids at the corresponding HR2 positions. See FIG. 10.

It was further tested whether the HR2 compensatory mutations improve the structural stability of these seven hairpin complexes. The results from residue-residue interaction and compensatory amino acid identification studies suggest that Q139R and/or S138Y mutations in the HR2 are likely to improve the RAPDF scores for these HR1 mutants. Therefore, the Q139R mutation was introduced in all the HR1 mutant structures with an additional S138Y mutation for the N42T+N43K and N42T+N43S mutant structures. It was found that these two HR2 compensatory mutations improved the RAPDF scores of all seven HR1 mutants (ranging from −36.21 to −35.02).

### Example 9

### Mechanism of Enfuvirtide Resistance

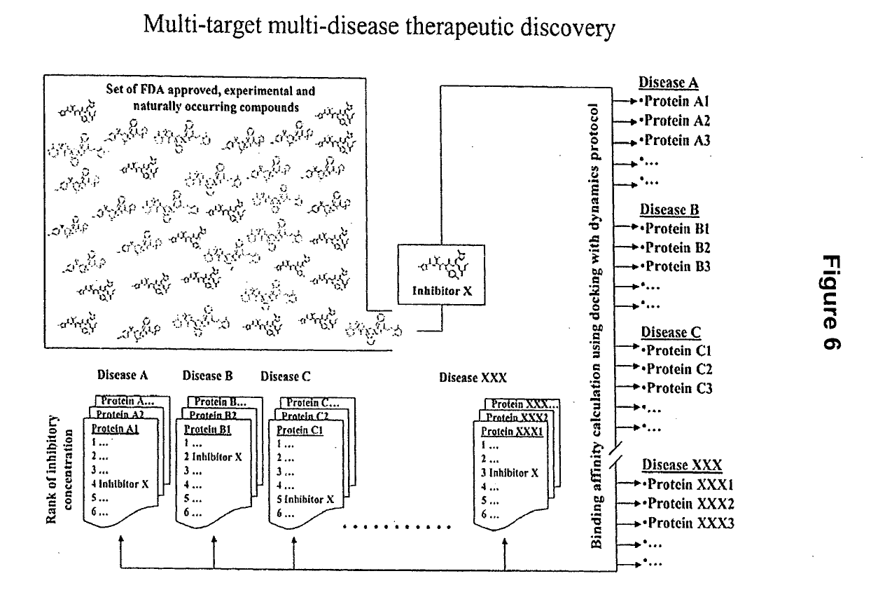

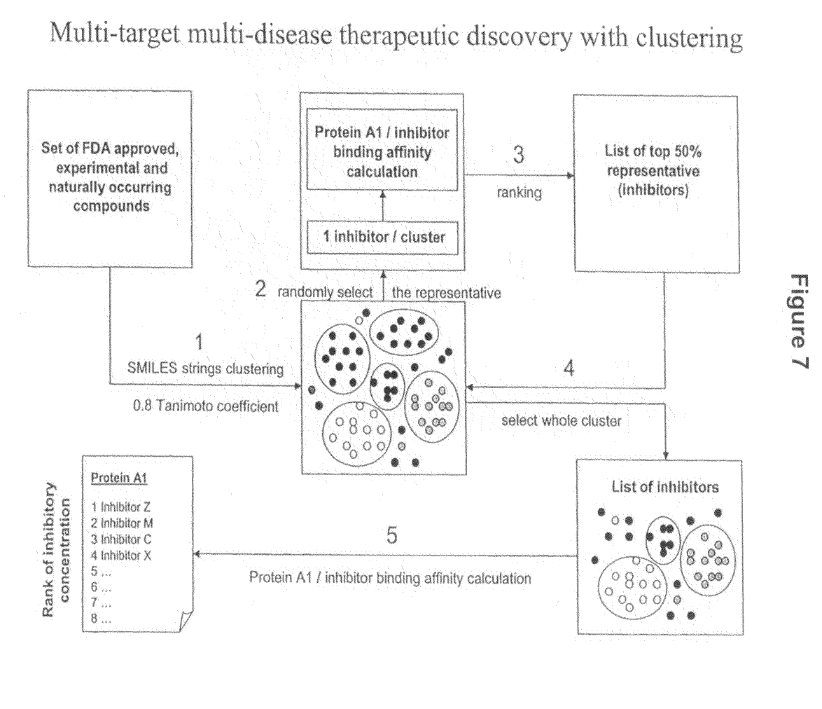

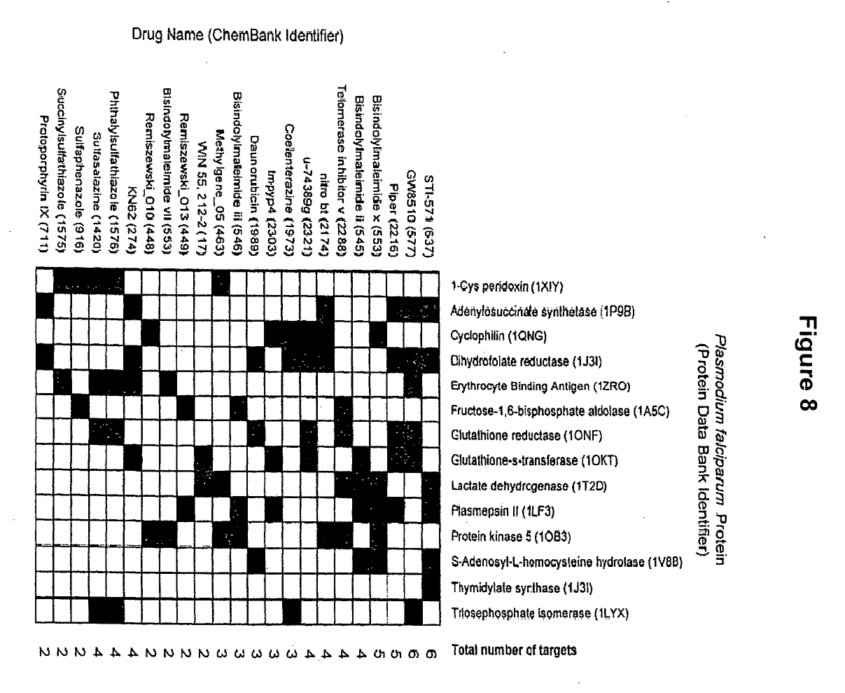

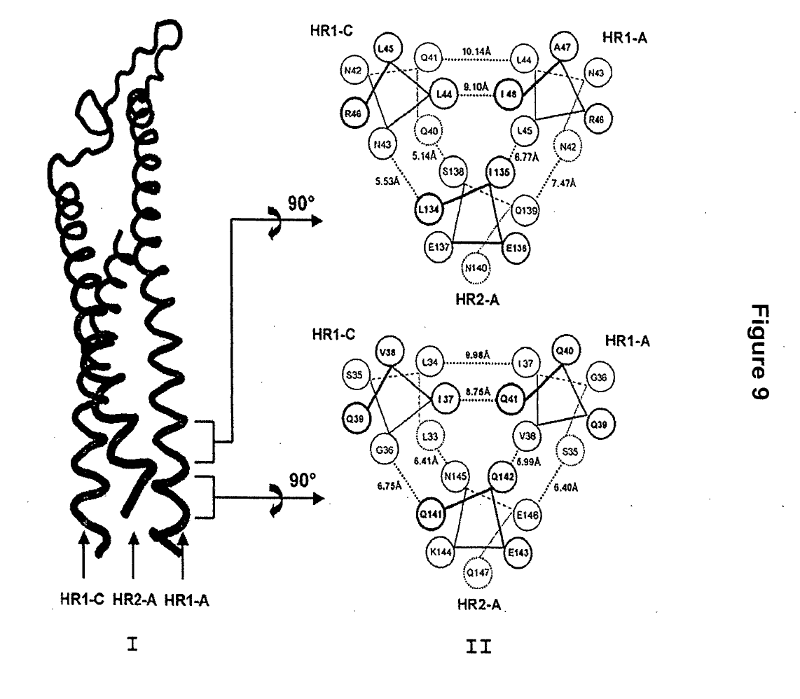

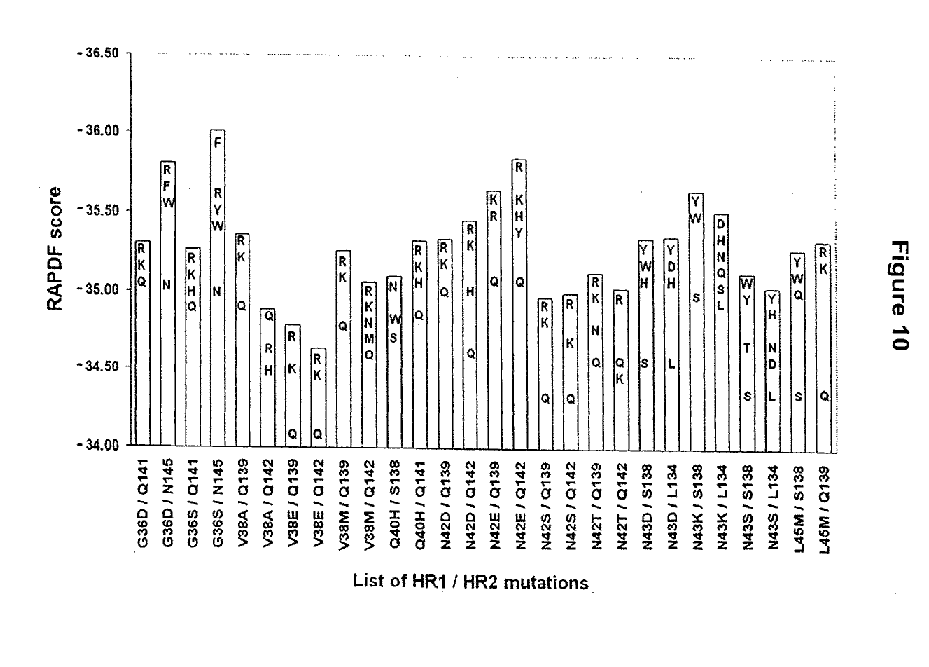

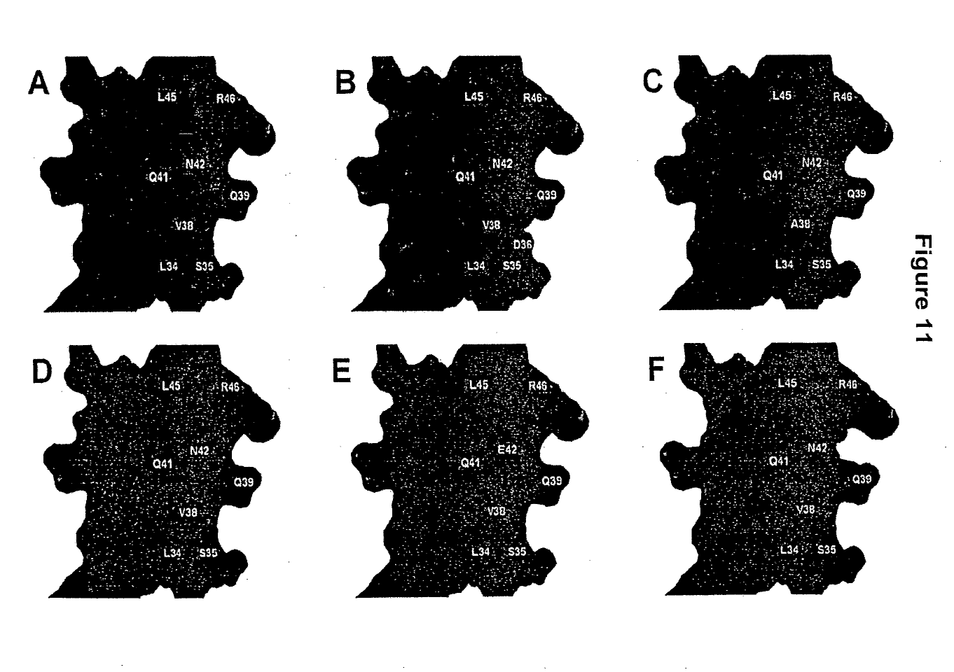

The theoretical structural stability scoring suggests a possible enfuvirtide-resistance mechanism: Initially, mutations in HR1 may be selected to reduce structural stability of HR1/enfuvirtide complex. These mutations alter the biochemical properties (for example, polarity and hydrophobicity) and modify the conformation of the HR1 coiled-coil hydrophobic groove. Comparison of the side chain arrangement at the binding surface of wild-type and mutant HR1 grooves shows that conformational changes are prominent in G36D, Q40H and N43K mutations. See FIG. 11. These changes limit binding site access of enfuvirtide resulting in increased EC₅₀ values. However, these mutants have low viral fitness because the mutations of the HR1 also reduce structural stability of the HR1/HR2 hairpin complex. Compensatory mutations at the corresponding HR2 residues are then selected to enhance structural stability of HR1/HR2 complex, thereby improving viral fitness and destabilizing the HR1/enfuvirtide complex.

FIG. 11 shows the surface structures of the hydrophobic groove formed by the HR1 domains of chain A and chain C. Comparison of the surface structures of the wild-type (A), G36D (B), V38A (C), Q40H (D), N42E (E) and N43K (F) mutants shows prominent changes at the HR1 grooves of G36D, Q40H and N43K mutants. The amino acids and numbers of HR1 chain A and chain C are labeled in yellow and red, respectively.

Even though we are unable to make a direct comparison between the increased theoretical stability of compensatory HR2 mutations and a corresponding increase in experimentally determined melting temperatures, we have demonstrated that the RAPDF score is a reliable indicator of melting temperatures for an independent set of mutations See Table 6. In addition, we have also shown that the RAPDF score is a reliable indicator of the EC₅₀ and viral fitness levels. See Table 7. Taken together, these results indicate that decreased stability of HR1 mutants (predicted by RAPDF scores that correlate well with experimentally-determined melting temperatures) is reversed by the compensatory HR2 mutants (as predicted by RAPDF), which in turn results in lower fusion inhibition and increased viral fitness (RAPDF scores also correlate well with these experimental measures of enfuvirtide activity). Our predictions and results are consistent with previously observed correlations between melting temperatures of the HR1/HR2 complex and fusion inhibition. Gallo et al., J Mol Biol 340: 9-14, 2004.

### Example 10

### Designing Enfuvirtide Derivatives Against Enfuvirtide-Resistant Strains

Previous experimental and clinical studies have identified eighteen mutation patterns in HR1 that are implicated in enfuvirtide resistance in patients [8-10]. In this study, six corresponding HR2 residues and the compensatory amino acids that play a role in improving structural stability of the enfuvirtide-resistant hairpin complex were identified. This suggests the possibility of designing enfuvirtide derivatives to inhibit these resistant strains.

To find the best derivative, eighteen enfuvirtide-resistant HR1 mutant structures were generated. For each HR1 mutant structure, the wild-type amino acids at six corresponding HR2 residues were randomly replaced by the compensatory amino acids yielding 27,440 HR1/HR2 mutant structures, each with a different HR2 mutation pattern. Finally, a total of 493,920 HR1/HR2 mutant structures were obtained after applying this procedure to all eighteen HR1 mutants.

Of the 493,920 mutant structures generated, a common HR2 mutation pattern was found that improved the RAPDF scores of all eighteen enfuvirtide-resistant strains, that is: L134H, S138Y, Q139R, Q141R, Q142R, N145Y/F/R. The scores ranged from −38.69 to −36.71, which were better than that of the HR1/HR2 wild-type strain. See Table 8. This finding suggests a list of the amino acids that may be used to design enfuvirtide derivatives. Modification of the enfuvirtide molecule should focus at the six corresponding residues with amino acid side chains replaced by the ones suggested in Table 8. Such a modification enhances interaction of the derivatives against the wild-type strain and the enfuvirtide-resistant mutants.

### Example 11

### Design of Peptidomimetic Inhibitors Against Drug Resistant Strains of Human Immunodeficiency Virus

In this study, a residue-specific all-atom probability discriminatory function (RAPDF) was applied to score theoretical models of HIV-1 gp41 to demonstrate that the mutant enfuvirtide-resistant strains have low structural stability. The RAPDF scores of these resistant mutants improved after amino acid substitutions at the corresponding residues of HR2 that interact with the HR1 mutant. The findings suggest an enfuvirtide resistance mechanism: Mutations of HR1 modify the hydrophobic groove that limits the likelihood of enfuvirtide accessing its binding site. Additional mutations at the corresponding HR2 residues improve structural stability of the HR1/HR2 hairpin complex, thereby enhancing viral fitness of the mutant strains. This resistance mechanism leads to the idea of designing novel enfuvirtide derivatives that may compete with the viral HR2 for binding in the modified HR1 hydrophobic groove. A combination of such enfuvirtide derivatives along with the HR1-derived enfuvirtide-complement peptide (and its derivatives) may have high potency in reducing viral load and have a wide spectrum effect in controlling HIV-1 wild-type as well as fusion inhibitor resistant strains.

### Example 12

### Broad Spectrum Inhibitors Against Infectious Disease

Binding energy and drug regimen prediction for protease inhibitors and HIV mutants has been completed. PIRSpred (protein inhibitor resistance/susceptibility prediction) software is available at http://protinfo.compbio.washington.edu/pirspred. The accuracy of the method is approximately 80% when used standalone and approximately 95% in combination with a knowledge based method when backtested. Jenwitheesuk E, Wang K, Mittler J, Samudrala R. Trends in Microbiology 13: 150-151, 2005; Jenwitheesuk E, Samudrala R., Antiviral Therapy 10: 157-166, 2005; Jenwitheesuk E, Wang K, Mittler J, Samudrala R., AIDS 18: 1858-1859, 2004; Wang K, Jenwitheesuk E, Samudrala R, Mittler J., Antiviral Therapy 9: 343-352, 2004.

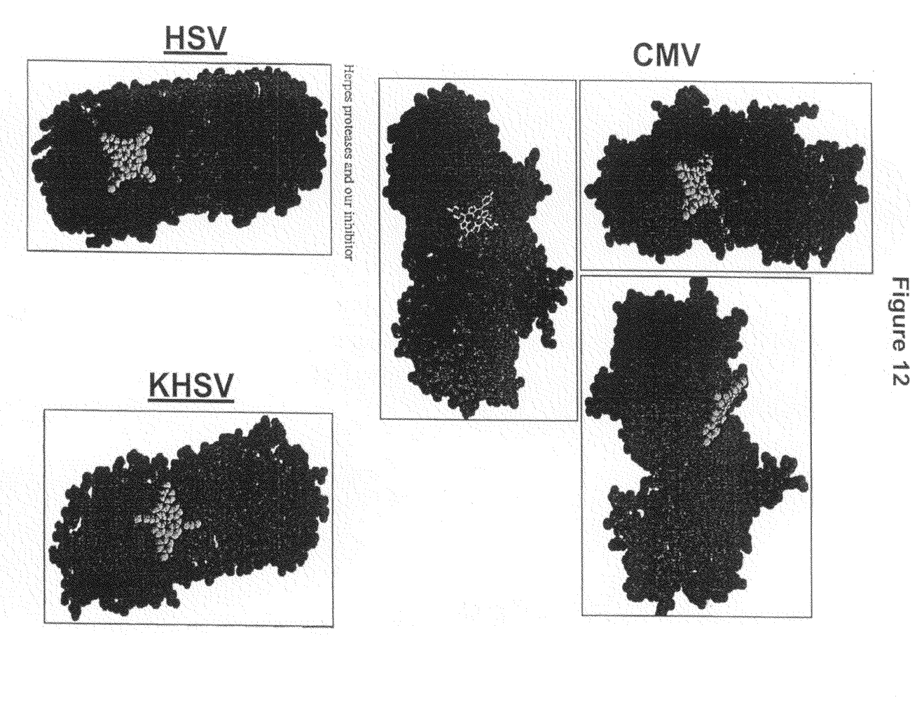

Broad-spectrum inhibitors against herpes simplex virus (HSV) proteases (including HSV2, VZV, EBV, CMV, and KSHV) have been studied. Docking with dynamics has been used as a general protocol to predict inhibitors from a pool of FDA experimental and approved drugs) against single targets in different herpesviruses. First round of predictions have been completed, resulting in up to 100 predicted leads. Two predicted inhibitors have been experimentally tested in vitro, and one inhibitor, TMPyP4, has demonstrated 90% inhibition of lytic phase viral growth in a non-toxic dose-dependent manner starting at micromolar concentrations. See Tables 9, 10, and 11. Three dimensional molecular modeling of the inhibitor, TMPyP4, bound to herpesvirus protease is shown in FIG. 12. Reference to “Drug ID” is in von Grotthuss, et al., “Ligand.Info Small-Molecule Meta-Database,” Comb Chem High Throughput Screen, 8: 757-761, 2004.

With regard to testing inhibitors predicted to be effective against herpesvirus infection, TMPyP4 was tested as the top prediction. The procedure for choosing the top prediction was as follows. The top 50 or top 100 list of drugs were chosen for all seeds against all proteins. One can then count how frequently each drug occurs and can rank each drug by its frequency. This final rank is used to suggest which inhibitors to test. For a candidate herpesvirus therapeutic composition, for example, TMPyP4 was the top ranking drug using this procedure.

Similarly, broad-spectrum inhibitors against HIV integrase have been studied. See Table 13. Reference to “Drug ID” is in von Grotthuss, et al., “Ligand.Info Small-Molecule Meta-Database,” Comb Chem High Throughput Screen, 8: 757-761, 2004.

Multi-target multi-condition inhibitors against multiple pathogens, such as inhibitors that target both HIV proteins as well as opportunistic infections that arise from HIV infection, for example, HIV and herpesviruses, or HIV and Pseudomonas. Predictions are in progress against targets from Pseudomonas aeruginosa, Pneumocystis carinii, Toxoplasma gondii, and Cryptosporidium. Natural outcome of other predictions (i.e., prediction of inhibitors against HIV integrase, protease, and herpesviruses). A study has shown predictions that HIV protease inhibitors also inhibit human cytomegalovirus protease. Jenwitheesuk E, and Samudrala R., AIDS 19: 529-533, 2005.

Predictions for broad-spectrum inhibitors are being studied against avian and dog influenza targets, e.g., protease.

HIV gp41 peptidomimetic inhibitors have been studied. Studies have shown that heptad-repeat-2 mutations enhance the stability of the enfuvirtide-resistant HIV-1 gp41 hairpin structure. Jenwitheesuk E, and Samudrala R., Antiviral Therapy 10: 893-900, 2005.

Predictions for broad-spectrum inhibitors are being studied against hepatitis virus.

Predictions for broad-spectrum inhibitors are being studied for inhibitors against proteins involved in cell cycle proliferation in brain cancer, e.g., medulloblastomas.

Predictions for multi-target Plasmodium falciparium inhibitors has been completed against 13 targets. Second round predictions including a 14th target are in progress. More than 20 FDA approved and experimental multi-target inhibitors have been identified against the 14 targets. Of the Plasmodium falciparium targets, Five to six known anti-malarial inhibitors rank in the top 5% in our lists of predictions of binding affinity.

Multi-target Trypanosoma and Leishmania inhibitor predictions are in progress for targets in Trypanosoma brucei, Trypanosoma cruzi, and Leishmania major (causing sleeping sickness, Chagas disease, and Leishmaniasis). A variety of human and animal diseases are caused by pathogens in these two genus, so drugs against these pathogens can be broadly effectively against these diseases.

SARS CoV protease inhibitors predicted that HIV protease inhibitors are effective against SARS CoV. It has been experimentally determined that HIV protease inhibitors are effective against SARS.

TABLE 9
Herpesvirus predicted inhibitors 1

| | Seed 1 | | | | | | | | | |
|---|---|---|---|---|---|---|---|---|---|---|
| | HSV2 | | VZV | | EBV | | CMV | | KSHV | |
| | Drug | Calculated | Drug | Calculated | Drug | Calculated | Drug | Calculated | Drug | Calculated |
| Rank | ID | Ki | ID | Ki | ID | Ki | ID | Ki | ID | Ki |
| 1 | 2216 | 1.20E−09 | 2216 | 3.40E−09 | 2287 | 3.38E−10 | 1607 | 1.93E−10 | 463 | 8.06E−09 |
| 2 | 845 | 9.47E−09 | 577 | 6.72E−09 | 2303 | 1.98E−09 | 2321 | 5.48E−10 | 759 | 1.04E−08 |
| 3 | 2303 | 1.25E−08 | 71 | 7.84E−09 | 577 | 4.65E−09 | 1089 | 7.07E−10 | 642 | 1.05E−08 |
| 4 | 759 | 1.30E−08 | 462 | 9.18E−09 | 429 | 7.30E−09 | 2303 | 8.19E−10 | 2321 | 2.23E−08 |
| 5 | 1576 | 1.37E−08 | 2321 | 1.25E−08 | 2263 | 8.44E−09 | 553 | 1.55E−09 | 1453 | 2.52E−08 |
| 6 | 1679 | 1.37E−08 | 1420 | 2.22E−08 | 2216 | 1.05E−08 | 436 | 3.15E−09 | 1984 | 3.98E−08 |
| 7 | 642 | 1.47E−08 | 1103 | 2.34E−08 | 1727 | 1.24E−08 | 1989 | 3.39E−09 | 2303 | 4.17E−08 |
| 8 | 1908 | 1.73E−08 | 463 | 2.35E−08 | 550 | 1.53E−08 | 2216 | 3.63E−09 | 1839 | 4.60E−08 |
| 9 | 1184 | 2.61E−08 | 1450 | 2.54E−08 | 27 | 1.63E−08 | 1570 | 7.00E−09 | 1721 | 4.94E−08 |
| 10 | 273 | 2.98E−08 | 1801 | 2.54E−08 | 1154 | 1.64E−08 | 1154 | 7.68E−09 | 845 | 5.04E−08 |
| 11 | 2277 | 3.00E−08 | 593 | 2.91E−08 | 350 | 1.82E−08 | 1 | 7.99E−09 | 535 | 6.23E−08 |
| 12 | 678 | 3.46E−08 | 1607 | 3.51E−08 | 2289 | 1.86E−08 | 2289 | 8.20E−09 | 581 | 6.68E−08 |
| 13 | 593 | 3.58E−08 | 545 | 3.57E−08 | 517 | 2.20E−08 | 550 | 8.94E−09 | 639 | 7.17E−08 |
| 14 | 552 | 3.72E−08 | 2288 | 4.64E−08 | 2232 | 2.20E−08 | 2009 | 9.26E−09 | 1731 | 7.18E−08 |
| 15 | 1648 | 3.81E−08 | 1184 | 5.14E−08 | 499 | 2.22E−08 | 2287 | 1.01E−08 | 553 | 7.41E−08 |
| 16 | 2135 | 4.51E−08 | 553 | 5.25E−08 | 590 | 2.32E−08 | 546 | 1.37E−08 | 2131 | 7.46E−08 |
| 17 | 702 | 4.80E−08 | 637 | 7.40E−08 | 1287 | 2.58E−08 | 151 | 1.38E−08 | 2289 | 7.81E−08 |
| 18 | 350 | 4.85E−08 | 1596 | 7.81E−08 | 639 | 2.60E−08 | 247 | 1.40E−08 | 534 | 9.34E−08 |
| 19 | 2009 | 5.03E−08 | 550 | 7.87E−08 | 637 | 2.65E−08 | 1585 | 1.46E−08 | 2232 | 9.38E−08 |
| 20 | 637 | 5.06E−08 | 1306 | 8.62E−08 | 463 | 2.69E−08 | 470 | 1.74E−08 | 551 | 9.73E−08 |
| 21 | 2134 | 5.22E−08 | 1585 | 8.72E−08 | 1973 | 2.71E−08 | 462 | 1.77E−08 | 637 | 1.06E−07 |
| 22 | 1338 | 5.37E−08 | 2289 | 8.72E−08 | 733 | 3.05E−08 | 702 | 1.87E−08 | 723 | 1.16E−07 |
| 23 | 1946 | 5.87E−08 | 1576 | 9.09E−08 | 1910 | 3.25E−08 | 2277 | 1.90E−08 | 2322 | 1.20E−07 |
| 24 | 663 | 6.37E−08 | 2308 | 9.12E−08 | 1917 | 3.34E−08 | 463 | 1.91E−08 | 549 | 1.21E−07 |
| 25 | 2141 | 6.60E−08 | 1795 | 9.56E−08 | 1680 | 3.45E−08 | 1721 | 1.99E−08 | 1727 | 1.28E−07 |
| 26 | 1834 | 7.84E−08 | 845 | 9.57E−08 | 488 | 3.49E−08 | 935 | 2.20E−08 | 469 | 1.38E−07 |
| 27 | 1944 | 8.29E−08 | 551 | 9.90E−08 | 624 | 3.51E−08 | 450 | 2.32E−08 | 2166 | 1.80E−07 |
| 28 | 553 | 8.61E−08 | 547 | 9.96E−08 | 586 | 3.61E−08 | 684 | 2.34E−08 | 232 | 1.86E−07 |
| 29 | 646 | 9.02E−08 | 546 | 1.07E−07 | 2288 | 3.61E−08 | 1910 | 2.57E−08 | 1801 | 1.86E−07 |
| 30 | 1569 | 9.26E−08 | 2174 | 1.09E−07 | 2174 | 3.66E−08 | 1921 | 2.79E−08 | 1629 | 1.94E−07 |
| 31 | 1596 | 9.52E−08 | 725 | 1.12E−07 | 1175 | 3.67E−08 | 1973 | 2.79E−08 | 586 | 1.95E−07 |
| 32 | 447 | 9.53E−08 | 1910 | 1.16E−07 | 702 | 3.90E−08 | 2232 | 2.87E−08 | 346 | 2.14E−07 |
| 33 | 872 | 9.56E−08 | 1164 | 1.17E−07 | 2329 | 4.01E−08 | 1666 | 3.16E−08 | 546 | 2.18E−07 |
| 34 | 1158 | 1.03E−07 | 468 | 1.18E−07 | 274 | 4.14E−08 | 2288 | 3.25E−08 | 1627 | 2.21E−07 |
| 35 | 2289 | 1.03E−07 | 639 | 1.24E−07 | 538 | 4.15E−08 | 548 | 3.32E−08 | 1582 | 2.27E−07 |
| 36 | 1154 | 1.07E−07 | 1672 | 1.24E−07 | 2009 | 4.35E−08 | 2136 | 3.33E−08 | 1778 | 2.31E−07 |
| 37 | 1420 | 1.09E−07 | 1679 | 1.34E−07 | 553 | 4.36E−08 | 1393 | 3.45E−08 | 1974 | 2.34E−07 |
| 38 | 151 | 1.25E−07 | 2303 | 1.39E−07 | 133 | 4.75E−08 | 577 | 3.67E−08 | 273 | 2.47E−07 |
| 39 | 2287 | 1.37E−07 | 131 | 1.43E−07 | 2321 | 4.79E−08 | 889 | 3.70E−08 | 550 | 2.57E−07 |
| 40 | 1164 | 1.39E−07 | 1418 | 1.60E−07 | 119 | 4.84E−08 | 567 | 3.95E−08 | 1785 | 2.57E−07 |
| 41 | 2174 | 1.46E−07 | 906 | 1.62E−07 | 1755 | 5.09E−08 | 591 | 4.11E−08 | 554 | 2.62E−07 |
| 42 | 445 | 1.57E−07 | 544 | 1.63E−07 | 606 | 5.17E−08 | 499 | 4.17E−08 | 1946 | 2.68E−07 |
| 43 | 468 | 1.57E−07 | 1705 | 1.63E−07 | 2092 | 5.18E−08 | 1287 | 4.30E−08 | 2092 | 2.72E−07 |
| 44 | 1240 | 1.60E−07 | 1629 | 1.76E−07 | 481 | 5.46E−08 | 574 | 4.58E−08 | 545 | 2.81E−07 |
| 45 | 2334 | 1.60E−07 | 460 | 1.85E−07 | 1075 | 5.89E−08 | 1450 | 4.63E−08 | 1261 | 2.83E−07 |
| 46 | 574 | 1.62E−07 | 552 | 2.03E−07 | 545 | 5.91E−08 | 238 | 4.67E−08 | 682 | 2.93E−07 |
| 47 | 1934 | 1.64E−07 | 119 | 2.07E−07 | 2065 | 5.94E−08 | 544 | 4.67E−08 | 593 | 2.96E−07 |
| 48 | 545 | 1.67E−07 | 273 | 2.12E−07 | 426 | 5.95E−08 | 2018 | 4.84E−08 | 1954 | 2.97E−07 |
| 49 | 461 | 1.70E−07 | 2092 | 2.15E−07 | 551 | 6.18E−08 | 447 | 4.95E−08 | 1156 | 2.99E−07 |
| 50 | 592 | 1.84E−07 | 503 | 2.18E−07 | 1596 | 6.47E−08 | 586 | 5.08E−08 | 621 | 3.03E−07 |

TABLE 10
Herpesvirus predicted inhibitors 2

| | Seed 2 | | | | | | | | | |
|---|---|---|---|---|---|---|---|---|---|---|
| | HSV2 | | VZV | | EBV | | CMV | | KSHV | |
| | Drug | Calculated | Drug | Calculated | Drug | Calculated | Drug | Calculated | Drug | Calculated |
| Rank | ID | Ki | ID | Ki | ID | Ki | ID | Ki | ID | Ki |
| 1 | 2322 | 2.98E−09 | 2216 | 1.84E−09 | 2303 | 2.43E−09 | 1607 | 2.15E−10 | 463 | 2.69E−09 |
| 2 | 2216 | 4.43E−09 | 577 | 5.95E−09 | 1585 | 3.23E−09 | 2303 | 3.66E−10 | 642 | 9.83E−09 |
| 3 | 577 | 6.55E−09 | 702 | 7.96E−09 | 2287 | 3.33E−09 | 2287 | 3.82E−10 | 1576 | 1.79E−08 |
| 4 | 642 | 8.72E−09 | 2321 | 1.24E−08 | 274 | 3.61E−09 | 1596 | 1.10E−09 | 1839 | 2.53E−08 |
| 5 | 2303 | 1.21E−08 | 1420 | 1.33E−08 | 681 | 3.71E−09 | 2216 | 5.17E−09 | 551 | 4.89E−08 |
| 6 | 1627 | 1.26E−08 | 463 | 1.44E−08 | 429 | 6.15E−09 | 151 | 5.23E−09 | 1629 | 5.09E−08 |
| 7 | 678 | 1.61E−08 | 1103 | 1.65E−08 | 2288 | 6.92E−09 | 247 | 5.36E−09 | 682 | 6.01E−08 |
| 8 | 759 | 1.62E−08 | 545 | 2.04E−08 | 577 | 7.48E−09 | 553 | 5.53E−09 | 639 | 6.52E−08 |
| 9 | 447 | 2.18E−08 | 1306 | 2.17E−08 | 2336 | 1.01E−08 | 1973 | 5.55E−09 | 1627 | 6.52E−08 |
| 10 | 1648 | 2.37E−08 | 2303 | 2.30E−08 | 462 | 1.20E−08 | 1154 | 6.09E−09 | 1984 | 6.81E−08 |
| 11 | 702 | 2.56E−08 | 1450 | 3.16E−08 | 1917 | 1.29E−08 | 448 | 6.27E−09 | 346 | 7.13E−08 |
| 12 | 2134 | 2.59E−08 | 551 | 3.51E−08 | 27 | 1.37E−08 | 2289 | 6.29E−09 | 553 | 8.26E−08 |
| 13 | 448 | 3.04E−08 | 2288 | 3.86E−08 | 1576 | 1.40E−08 | 546 | 6.93E−09 | 2322 | 8.57E−08 |
| 14 | 1946 | 3.61E−08 | 449 | 4.10E−08 | 544 | 1.56E−08 | 2009 | 7.11E−09 | 2232 | 1.04E−07 |
| 15 | 2141 | 4.00E−08 | 462 | 4.37E−08 | 2216 | 1.66E−08 | 1065 | 9.39E−09 | 2321 | 1.05E−07 |
| 16 | 350 | 4.28E−08 | 1596 | 4.50E−08 | 517 | 2.00E−08 | 1585 | 1.32E−08 | 71 | 1.07E−07 |
| 17 | 1184 | 4.59E−08 | 1585 | 4.54E−08 | 1658 | 2.00E−08 | 1910 | 1.37E−08 | 550 | 1.10E−07 |
| 18 | 273 | 4.76E−08 | 1801 | 4.85E−08 | 350 | 2.48E−08 | 342 | 1.45E−08 | 637 | 1.16E−07 |
| 19 | 2336 | 4.94E−08 | 546 | 4.91E−08 | 661 | 2.55E−08 | 2288 | 1.45E−08 | 554 | 1.18E−07 |
| 20 | 1596 | 5.09E−08 | 1834 | 4.98E−08 | 1727 | 2.78E−08 | 893 | 1.46E−08 | 586 | 1.18E−07 |
| 21 | 1834 | 5.50E−08 | 593 | 5.19E−08 | 2289 | 2.84E−08 | 1089 | 1.57E−08 | 2289 | 1.18E−07 |
| 22 | 593 | 5.99E−08 | 1576 | 5.31E−08 | 1607 | 3.16E−08 | 1658 | 1.67E−08 | 549 | 1.22E−07 |
| 23 | 1785 | 6.48E−08 | 1089 | 5.32E−08 | 1973 | 3.17E−08 | 2321 | 1.69E−08 | 334 | 1.32E−07 |
| 24 | 2277 | 6.55E−08 | 548 | 6.02E−08 | 1075 | 3.22E−08 | 550 | 1.73E−08 | 546 | 1.34E−07 |
| 25 | 1576 | 6.73E−08 | 1672 | 6.73E−08 | 2172 | 3.41E−08 | 499 | 1.75E−08 | 390 | 1.41E−07 |
| 26 | 538 | 7.43E−08 | 1607 | 6.76E−08 | 1 | 3.43E−08 | 1569 | 1.82E−08 | 17 | 1.46E−07 |
| 27 | 551 | 7.83E−08 | 550 | 6.81E−08 | 639 | 3.66E−08 | 2232 | 1.87E−08 | 484 | 1.48E−07 |
| 28 | 553 | 8.01E−08 | 1184 | 7.17E−08 | 1524 | 3.74E−08 | 1989 | 1.98E−08 | 1974 | 1.68E−07 |
| 29 | 1270 | 8.02E−08 | 639 | 7.58E−08 | 1989 | 3.75E−08 | 577 | 2.01E−08 | 1075 | 1.87E−07 |
| 30 | 637 | 8.63E−08 | 469 | 8.36E−08 | 590 | 3.85E−08 | 591 | 2.03E−08 | 3 | 1.88E−07 |
| 31 | 1585 | 8.74E−08 | 131 | 9.06E−08 | 1963 | 3.92E−08 | 1721 | 2.17E−08 | 2205 | 1.92E−07 |
| 32 | 468 | 9.04E−08 | 1807 | 9.34E−08 | 1910 | 3.93E−08 | 2018 | 2.20E−08 | 1453 | 1.96E−07 |
| 33 | 501 | 9.08E−08 | 544 | 9.36E−08 | 2135 | 3.93E−08 | 1450 | 2.21E−08 | 1727 | 2.09E−07 |
| 34 | 872 | 9.50E−08 | 484 | 9.62E−08 | 889 | 4.04E−08 | 447 | 2.41E−08 | 545 | 2.15E−07 |
| 35 | 1164 | 9.61E−08 | 845 | 1.03E−07 | 2329 | 4.15E−08 | 725 | 2.53E−08 | 604 | 2.18E−07 |
| 36 | 463 | 1.08E−07 | 606 | 1.11E−07 | 591 | 4.52E−08 | 462 | 2.65E−08 | 1156 | 2.27E−07 |
| 37 | 552 | 1.12E−07 | 1164 | 1.18E−07 | 471 | 4.91E−08 | 586 | 2.87E−08 | 1731 | 2.33E−07 |
| 38 | 893 | 1.12E−07 | 1584 | 1.19E−07 | 310 | 4.92E−08 | 2058 | 2.87E−08 | 759 | 2.36E−07 |
| 39 | 462 | 1.15E−07 | 1909 | 1.20E−07 | 9 | 5.04E−08 | 2277 | 2.91E−08 | 1946 | 2.37E−07 |
| 40 | 479 | 1.30E−07 | 1598 | 1.23E−07 | 1710 | 5.25E−08 | 574 | 2.92E−08 | 1705 | 2.41E−07 |
| 41 | 248 | 1.39E−07 | 2287 | 1.25E−07 | 492 | 5.48E−08 | 818 | 2.95E−08 | 1000 | 2.42E−07 |
| 42 | 725 | 1.39E−07 | 1648 | 1.41E−07 | 1390 | 5.64E−08 | 551 | 2.97E−08 | 2092 | 2.42E−07 |
| 43 | 1154 | 1.44E−07 | 2141 | 1.43E−07 | 939 | 5.76E−08 | 1548 | 3.02E−08 | 1295 | 2.44E−07 |
| 44 | 461 | 1.45E−07 | 2092 | 1.49E−07 | 1184 | 5.80E−08 | 1576 | 3.16E−08 | 1582 | 2.46E−07 |
| 45 | 2287 | 1.45E−07 | 931 | 1.50E−07 | 1620 | 5.81E−08 | 889 | 3.17E−08 | 678 | 2.52E−07 |
| 46 | 197 | 1.47E−07 | 1705 | 1.52E−07 | 702 | 5.85E−08 | 436 | 3.35E−08 | 1954 | 2.53E−07 |
| 47 | 554 | 1.49E−07 | 684 | 1.54E−07 | 2065 | 5.87E−08 | 450 | 3.46E−08 | 538 | 2.75E−07 |
| 48 | 586 | 1.52E−07 | 468 | 1.60E−07 | 1705 | 6.04E−08 | 1 | 3.60E−08 | 1663 | 2.75E−07 |
| 49 | 151 | 1.56E−07 | 839 | 1.62E−07 | 2308 | 6.14E−08 | 1455 | 3.70E−08 | 2216 | 2.82E−07 |
| 50 | 1158 | 1.56E−07 | 120 | 1.64E−07 | 151 | 6.51E−08 | 711 | 3.72E−08 | 845 | 2.84E−07 |

TABLE 11
Herpesvirus predicted inhibitors 3

| | Seed 3 | | | | | | | | | |
|---|---|---|---|---|---|---|---|---|---|---|
| | HSV2 | | VZV | | EBV | | CMV | | KSHV | |
| | Drug | Calculated | Drug | Calculated | Drug | Calculated | Drug | Calculated | Drug | Calculated |
| Rank | ID | Ki | ID | Ki | ID | Ki | ID | Ki | ID | Ki |
| 1 | 2216 | 8.16E−09 | 1721 | 1.27E−08 | 2303 | 3.20E−09 | 1607 | 3.87E−10 | 642 | 1.32E−08 |
| 2 | 642 | 1.69E−08 | 2303 | 1.49E−08 | 550 | 5.34E−09 | 553 | 1.29E−09 | 845 | 1.50E−08 |
| 3 | 2303 | 2.89E−08 | 2321 | 1.89E−08 | 952 | 5.65E−09 | 2303 | 1.54E−09 | 639 | 2.37E−08 |
| 4 | 553 | 4.39E−08 | 2216 | 2.18E−08 | 577 | 9.29E−09 | 2216 | 5.71E−09 | 2303 | 3.49E−08 |
| 5 | 702 | 1.15E−07 | 71 | 2.34E−08 | 429 | 1.32E−08 | 1154 | 6.05E−09 | 702 | 4.58E−08 |
| 6 | 151 | 1.42E−07 | 1420 | 2.34E−08 | 350 | 2.23E−08 | 436 | 8.26E−09 | 551 | 5.74E−08 |
| 7 | 2322 | 1.50E−07 | 1801 | 2.58E−08 | 1917 | 2.40E−08 | 247 | 8.90E−09 | 1727 | 5.94E−08 |
| 8 | 1240 | 1.66E−07 | 1103 | 2.81E−08 | 624 | 2.97E−08 | 2287 | 9.26E−09 | 553 | 6.13E−08 |
| 9 | 448 | 1.69E−07 | 1306 | 3.18E−08 | 2321 | 3.87E−08 | 2321 | 1.14E−08 | 535 | 6.82E−08 |
| 10 | 577 | 1.75E−07 | 545 | 3.29E−08 | 2232 | 4.25E−08 | 546 | 1.26E−08 | 1839 | 6.86E−08 |
| 11 | 2174 | 1.82E−07 | 463 | 3.90E−08 | 362 | 4.45E−08 | 2232 | 1.58E−08 | 346 | 6.91E−08 |
| 12 | 1596 | 1.92E−07 | 577 | 4.36E−08 | 2329 | 4.93E−08 | 551 | 1.70E−08 | 545 | 8.23E−08 |
| 13 | 872 | 2.09E−07 | 27 | 5.65E−08 | 1000 | 5.99E−08 | 2301 | 1.81E−08 | 550 | 8.26E−08 |
| 14 | 678 | 2.18E−07 | 1672 | 5.67E−08 | 1658 | 6.33E−08 | 550 | 1.89E−08 | 637 | 1.04E−07 |
| 15 | 1158 | 2.23E−07 | 2287 | 6.56E−08 | 574 | 6.43E−08 | 462 | 1.95E−08 | 1984 | 1.06E−07 |
| 16 | 2134 | 2.38E−07 | 2308 | 6.66E−08 | 1910 | 6.44E−08 | 450 | 2.11E−08 | 549 | 1.25E−07 |
| 17 | 247 | 2.51E−07 | 1910 | 6.82E−08 | 2065 | 7.35E−08 | 586 | 2.16E−08 | 1974 | 1.25E−07 |
| 18 | 586 | 2.90E−07 | 1576 | 6.87E−08 | 1989 | 7.36E−08 | 1 | 2.32E−08 | 2322 | 1.31E−07 |
| 19 | 548 | 2.99E−07 | 462 | 7.04E−08 | 749 | 7.53E−08 | 554 | 2.50E−08 | 586 | 1.35E−07 |
| 20 | 545 | 3.09E−07 | 2277 | 8.27E−08 | 1727 | 7.76E−08 | 448 | 2.83E−08 | 1629 | 1.43E−07 |
| 21 | 463 | 3.26E−07 | 1807 | 8.90E−08 | 151 | 7.80E−08 | 2018 | 3.11E−08 | 2232 | 1.50E−07 |
| 22 | 1064 | 3.27E−07 | 469 | 8.91E−08 | 635 | 8.24E−08 | 151 | 3.13E−08 | 1954 | 1.54E−07 |
| 23 | 1679 | 3.31E−07 | 639 | 9.44E−08 | 119 | 8.60E−08 | 1418 | 3.27E−08 | 2321 | 1.60E−07 |
| 24 | 1631 | 3.50E−07 | 546 | 9.63E−08 | 586 | 8.61E−08 | 2136 | 3.43E−08 | 546 | 1.62E−07 |
| 25 | 1724 | 3.51E−07 | 1089 | 9.65E−08 | 423 | 8.67E−08 | 1922 | 4.05E−08 | 2018 | 1.80E−07 |
| 26 | 245 | 3.53E−07 | 1164 | 1.08E−07 | 1014 | 8.74E−08 | 670 | 4.25E−08 | 635 | 1.92E−07 |
| 27 | 282 | 3.67E−07 | 548 | 1.18E−07 | 557 | 8.98E−08 | 470 | 5.27E−08 | 604 | 2.02E−07 |
| 28 | 460 | 3.90E−07 | 552 | 1.23E−07 | 426 | 9.04E−08 | 463 | 5.42E−08 | 547 | 2.13E−07 |
| 29 | 160 | 3.95E−07 | 906 | 1.23E−07 | 9 | 9.15E−08 | 818 | 5.42E−08 | 445 | 2.20E−07 |
| 30 | 17 | 4.23E−07 | 916 | 1.33E−07 | 591 | 9.84E−08 | 2009 | 5.50E−08 | 544 | 2.37E−07 |
| 31 | 1767 | 4.25E−07 | 1450 | 1.37E−07 | 247 | 1.00E−07 | 1431 | 5.65E−08 | 552 | 2.51E−07 |
| 32 | 1801 | 4.54E−07 | 1184 | 1.40E−07 | 1175 | 1.01E−07 | 274 | 5.69E−08 | 1270 | 2.73E−07 |
| 33 | 2092 | 4.70E−07 | 684 | 1.45E−07 | 544 | 1.02E−07 | 637 | 5.78E−08 | 484 | 2.85E−07 |
| 34 | 1576 | 4.74E−07 | 1705 | 1.54E−07 | 678 | 1.05E−07 | 2263 | 6.12E−08 | 436 | 3.07E−07 |
| 35 | 725 | 4.75E−07 | 2289 | 1.55E−07 | 1922 | 1.05E−07 | 538 | 6.49E−08 | 1223 | 3.08E−07 |
| 36 | 468 | 4.86E−07 | 725 | 1.58E−07 | 642 | 1.15E−07 | 603 | 7.01E−08 | 548 | 3.09E−07 |
| 37 | 552 | 4.92E−07 | 839 | 1.58E−07 | 1537 | 1.17E−07 | 1103 | 7.09E−08 | 621 | 3.14E−07 |
| 38 | 1651 | 4.94E−07 | 460 | 1.59E−07 | 1829 | 1.19E−07 | 725 | 7.24E−08 | 2092 | 3.26E−07 |
| 39 | 287 | 4.99E−07 | 2141 | 1.64E−07 | 637 | 1.21E−07 | 577 | 7.97E−08 | 1295 | 3.34E−07 |
| 40 | 2047 | 5.01E−07 | 131 | 1.70E−07 | 804 | 1.21E−07 | 711 | 8.17E−08 | 725 | 3.47E−07 |
| 41 | 637 | 5.13E−07 | 581 | 1.75E−07 | 310 | 1.25E−07 | 2277 | 8.81E−08 | 813 | 3.47E−07 |
| 42 | 1589 | 5.13E−07 | 550 | 1.76E−07 | 1322 | 1.33E−07 | 447 | 9.05E−08 | 1377 | 3.51E−07 |
| 43 | 40 | 5.28E−07 | 1629 | 1.78E−07 | 553 | 1.40E−07 | 238 | 9.25E−08 | 1548 | 3.52E−07 |
| 44 | 329 | 5.35E−07 | 1956 | 1.79E−07 | 1492 | 1.41E−07 | 1917 | 9.54E−08 | 463 | 3.55E−07 |
| 45 | 2330 | 5.36E−07 | 538 | 2.09E−07 | 446 | 1.43E−07 | 1393 | 9.66E−08 | 2216 | 3.62E−07 |
| 46 | 1147 | 5.48E−07 | 591 | 2.23E−07 | 724 | 1.44E−07 | 1721 | 9.69E−08 | 1733 | 3.64E−07 |
| 47 | 133 | 5.65E−07 | 1131 | 2.24E−07 | 2249 | 1.44E−07 | 723 | 9.79E−08 | 1919 | 3.72E−07 |
| 48 | 1388 | 5.85E−07 | 1295 | 2.31E−07 | 469 | 1.45E−07 | 2308 | 9.90E−08 | 1037 | 4.41E−07 |
| 49 | 2232 | 5.85E−07 | 3 | 2.32E−07 | 2288 | 1.48E−07 | 823 | 9.96E−08 | 390 | 4.42E−07 |
| 50 | 1607 | 6.26E−07 | 325 | 2.33E−07 | 2092 | 1.50E−07 | 2289 | 1.01E−07 | 1 | 4.43E−07 |

### Example 13

### Virtual Screening of HIV-1 Protease Inhibitors Against Human Cytomegalovirus Protease Using Docking and Molecular Dynamics

The use of docking with dynamics has been used to predict effectiveness of HIV protease inhibitors against CMV protease. Clearance of CMV viraemia in HIV-1 infected patients may result in part from inhibition of CMV protease by HIV-1 protease inhibitors contained in HAART. A computational method has been used to calculate the binding affinity of six HIV-1 protease inhibitors to CMV protease based on its x-ray crystallography structure. The calculations show that amprenavir and indinavir occupy the substrate-binding site of the CMV protease with high affinity and may be implicated in alleviating CMV infection. Cytomegalovirus (CMV) is an AIDS-related opportunistic pathogen that usually infects human immunodeficiency virus type 1 (HIV-1) patients with high level of plasma HIV-1 RNA and low CD4 counts (<200 cells/μL). Mentec et al., AIDS 8: 461-467, 1994; Alder et al., Ophthalmology 105: 651-657, 1998; Gellrich et al., Br J Ophthalmol 80: 818-822, 1996. Highly active antiretroviral therapy (HAART), consisting of HIV-1 protease and reverse transcriptase inhibitors, has been shown to lower plasma HIV-1 RNA levels and elevate CD4 cell counts, and is associated with a reduction in CMV replication and clearance of CMV viraemia. Macdonald et al., J Infect Dis 177: 1182-1187, 1998; Vrabec et al., Ophthalmology 105: 1259-1264, 1998; Casado et al., J Acquir Immune Defic Syndr Hum Retrovirol 19: 130-134, 1998; Jabs et al., Am J Ophthamol 126: 817-822, 1998; Deayton et al., AIDS 13: 1203-1206, 1999; Casado et al., AIDS 13: 1497-1502, 1999; Macdonald et al., Ophthalmology 107: 877-881, 2000; Reed et al., Retina 21: 339-343, 2001. Reports from several groups have shown that immune recovery that results from HAART without any specific anti-CMV therapy is able to suppress CMV infection in HIV-1 infected patients. Deayton et al., AIDS 13: 1203-1206, 1999; Casado et al., AIDS 13: 1497-1502, 1999; Macdonald et al., Ophthalmology 107: 877-881, 2000; Reed et al., Retina 21: 339-343, 2001. However, it is unresolved as to whether HIV-1 protease inhibitors aid clearance of CMV viraemia by inhibiting CMV protease activity.

In this study, an integrated molecular dynamics (MD) simulation and docking methods was used to calculate the ability of six Food and Drug Administration (FDA) approved HIV-1 protease inhibitors to bind to the CMV protease in terms of binding mode and binding energy. The x-ray crystallography structures of CMV protease and HIV-1 protease inhibitors were retrieved from the Protein Data Bank (PDB) (PDB codes: 1NKM for CMV protease, 1HPV for amprenavir, 1HSG for indinavir, 1MUI for lopinavir, 1OHR for nelfinavir, 1HXW for ritonavir and 1C6Z for saquinavir).

Docking calculations were carried out using AutoDock version 3.0.5 with a Lamarckian genetic algorithm. Morris et al, J Comput Chem 19: 1639-1662, 1998. Preliminary docking experiments were first performed to identify the potential binding sites of the inhibitors by generating a grid box that is big enough to cover the entire surface of the protein. The protein-inhibitor complexes derived from the first ranked docking solution in the preliminary docking procedure were consequently solvated in a TIP3-water shell and all atoms were allowed to relax using MD simulation. The MD simulation was carried out with the NAMD software version 2.5b18. Kale et al., J Comput Phys 151: 283-312, 1999. The topology and parameters for each inhibitor was obtained from the PRODRG server. van Aalten et al., J Comput Aided Mol Des 10: 255-262, 1996. One hundred steps of energy minimization of the protein-inhibitor-water complex were initially performed, followed by 0.1 picoseconds of MD simulation at 300 K. The simulations were repeated with three different starting seeds. The trajectories at 0.1 picoseconds were recorded and processed in a second docking step using similar docking parameters as used in the preliminary docking procedure. The primary exception was in the creation of a 3D affinity grid box, where the C-α atom of Ser132 of the catalytic triad was set as a grid center, and the number of grid points in the x, y, z-axes was set to 60×60×60.

AutoDock generates three energy terms: intermolecular energy, internal energy of the ligand, and torsional free energy. The final docked energy was calculated from the sum of the intermolecular energy and the internal energy of the ligand. The free energy of binding was calculated from the sum of the intermolecular and the torsional free energies, and consequently converted into an inhibitory constant (Kᵢ) according to Hess's law. The lowest-energy solution was accepted as the calculated binding energy and its Kᵢ value was used to define the binding affinity of the inhibitors. Further details of our MD simulation and docking protocols are given elsewhere. Jenwitheesuk et al., BMC Struct Biol 3: 2, 2003; Jenwitheesuk et al., Bioorg Med Chem Lett 13: 3989-3992, 2003.

Structural studies of the CMV protease show that it belongs to the serine protease family, with a novel Ser132-His63-His157 catalytic triad, with His157 representing the third member instead of the typical Asp. Tong et al., Nat Struct Biol 5: 819-826, 1998. The substrate-binding site is composed of several subsites: The S₁ subsite is formed by residues Leu32, Ser132, Leu133, Arg165 and Arg166. The S₁ and S₄ subsites are fused together, forming a large pocket with residues His63 and Asp64 on one side, Ser135 on the other, and Lys156 in the middle. The S₃ portion of this pocket is formed by salt bridges between residues Glu31, Ser135, Arg137, Arg165 and Arg166. Tong et al., Nat Struct Biol 5: 819-826, 1998. Theoretically, enzymatic activity would be significantly diminished if the catalytic triad, or part of the substrate-binding sites, were occupied by a small drug molecule or peptidomimetic inhibitor.

The first ranked docking solution derived from the preliminary docking procedure showed that all inhibitors bound to the substrate-binding site of the CMV protease. The binding energy and the calculated Kᵢ obtained after MD simulation and second round docking showed that amprenavir and indinavir had high affinity for the CMV protease (as indicated by calculated Kᵢ<10⁻⁸ and final docked energy <−14.00 kcal/mol) with the inhibitor occupying subsites S₁, S₂ and S₃. See Table 12. The other four inhibitors, lopinavir, nelfinavir, ritonavir and saquinavir, only partially fit into one or two subsites. The docked energy, the calculated Kᵢ and the binding modes of nelfinavir and lopinavir indicated that these two inhibitors bound the CMV protease more weakly than the other inhibitors. Identical results were obtained for all the three starting seeds used.

A number of studies suggest that protease inhibitors included in the HAART regimen have had a significant impact on CMV infection in decreasing the incidence, changing clinical course, and altering clinical presentation. Macdonald et al., J Infect Dis 177: 1182-1187, 1998; Vrabec et al., Ophthalmology 105: 1259-1264, 1998; Casado et al., J Acquir Immune Defic Syndr Hum Retrovirol 19: 130-134, 1998; Jabs et al., Am J Ophthalmol 126: 817-822, 1998; Deayton et al., AIDS 13: 1203-1206, 1999; Casado et al., AIDS 13: 1497-1502, 1999; Macdonald et al., Ophthalmology 107: 877-881, 2000; Reed et al., Retina 21: 339-343, 2001. However, none of them have identified the inhibitory activity of individual HIV-1 protease inhibitors against CMV.

This computational study provides evidence for the inhibitory activity of two approved inhibitors, amprenavir and indinavir, against the CMV protease. Including either of these two inhibitors in HAART regimen should help control the CMV viral load in HIV-1 infected patients. The activity of the CMV protease would be inhibited soon after starting HAART, in contrast to inhibition by promoting immune system restoration, which may take several weeks.

This study also provides a list of candidate inhibitors that may be experimentally tested for CMV protease inhibitory activity, and for further design of broad-spectrum inhibitors, to control both HIV-1 and CMV infection. Structural studies of human herpes proteases (of which CMV is one) indicate homology among several subtypes. Holwerda, Antiviral Res 35: 1-21, 1997; Qiu et al., Proc Natl Acad Sci USA 94: 2874-2879, 1997; Buisson et al., J Mol Biol 324: 89-103, 2002. Thus further studies to investigate the interaction and activity of these inhibitors, including approved drugs, against proteases from human herpesviruses may be fruitful in combating opportunistic infections originating in HIV-1 patients.

TABLE 12
Calculated energies and inhibitory constants (Kᵢ) of six FDA approved HIV-1 protease
inhibitors against the CMV protease ranked in ascending order of calculated Kᵢ.
Amprenavir and indinavir have high affinity for the CMV protease, with a final docked
energy <−14.00 kcal/mol and calculated Kᵢ < 1 × 10⁻⁸.

| | | Intermolecular | Internal energy | Torsional | Final docked | Calculated |
|---|---|---|---|---|---|---|
| PDB | | energy | of ligand | free energy | energy | inhibitory |
| ID | Inhibitor | (kcal/mol) | (kcal/mol) | (kcal/mol) | (kcal/mol) | constant (Kᵢ) |
| 1HPV | Amprenavir | −15.43 | −0.27 | 4.36 | −15.70 | 7.66 × 10⁻⁹ |
| 1HSG | Indinavir | −15.31 | 1.05 | 4.36 | −14.26 | 9.37 × 10⁻⁹ |
| 1C6Z | Saquinavir | −15.02 | 0.80 | 4.98 | −14.22 | 4.35 × 10⁻⁸ |
| 1HXW | Ritonavir | −15.62 | −0.37 | 6.85 | −15.99 | 3.69 × 10⁻⁷ |
| 1OHR | Nelfinavir | −12.23 | −0.64 | 3.74 | −12.87 | 5.93 × 10⁻⁷ |
| 1MUI | Lopinavir | −13.45 | 0.02 | 5.60 | −13.43 | 1.76 × 10⁻⁶ |

### Example 14

### Virtual Screening of Multitarget Small Chemical Molecule Inhibitors of HIV-1 Targeting HIV-1 Integrase and TAR Using Docking and Molecular Dynamics

Table 13 shows the results of screening broad spectrum small molecule chemical inhibitors against HIV-1 integrase and TAR. The inhibitors with the highest predicted activity against HIV-1 integrase include, but are not limited to, TMPyP4 (2303), calmidazolium chloride (1951), paromomycin (1565), aurintricarboxylic acid (1921), ro 31-8220 (548), Dichlorobenzamil (36), catenulin (1198), kanamycin (670), and capreomycin (893).

### Example 15

### Virtual Screening of Multitarget Small Chemical Molecule Inhibitors of HIV-1 Targeting HIV-1 Capsid Using Docking and Molecular Dynamics

Table 14 shows multitarget small molecule inhibitors of HIV-1 and predicted inhibitors of HIV-1 capsid.

### Example 16

### Virtual Screening of Multitarget Small Chemical Molecule Inhibitors of Mycobacterium tuberculosis Using Docking and Molecular Dynamics

Table 15 shows multitarget small molecule inhibitors of Mycobacterium tuberculosis.

TABLE 13
Predicted Inhibitors for HIV-1 Integrase and TAR

| | Seed 1 | | Seed 2 | | Seed 3 | |
|---|---|---|---|---|---|---|
| Rank | Drug ID | Ki | Drug ID | Ki | Drug ID | Ki |
| 1 | 2303 | 4.65E−10 | 2303 | 4.65E−10 | 2303 | 4.63E−10 |
| 2 | 1951 | 6.35E−09 | 1565 | 9.12E−10 | 1921 | 4.78E−09 |
| 3 | 1565 | 7.12E−09 | 1921 | 5.06E−09 | 1951 | 6.14E−09 |
| 4 | 1921 | 8.72E−09 | 1951 | 6.69E−09 | 1198 | 6.59E−09 |
| 5 | 1198 | 1.04E−08 | 548 | 1.45E−08 | 548 | 1.48E−08 |
| 6 | 670 | 2.02E−08 | 1117 | 2.08E−08 | 1565 | 1.87E−08 |
| 7 | 893 | 2.14E−08 | 685 | 2.49E−08 | 670 | 2.30E−08 |
| 8 | 548 | 2.78E−08 | 919 | 3.13E−08 | 893 | 2.41E−08 |
| 9 | 1469 | 3.35E−08 | 1462 | 3.14E−08 | 36 | 3.46E−08 |
| 10 | 36 | 3.51E−08 | 36 | 3.32E−08 | 482 | 3.48E−08 |
| 11 | 482 | 3.52E−08 | 553 | 3.45E−08 | 1570 | 3.51E−08 |
| 12 | 685 | 3.57E−08 | 482 | 3.62E−08 | 553 | 3.69E−08 |
| 13 | 553 | 3.69E−08 | 1570 | 4.12E−08 | 1462 | 4.30E−08 |
| 14 | 1570 | 3.99E−08 | 1469 | 4.40E−08 | 552 | 4.78E−08 |
| 15 | 1462 | 4.24E−08 | 552 | 4.68E−08 | 1469 | 5.24E−08 |
| 16 | 552 | 5.46E−08 | 2089 | 5.49E−08 | 1117 | 5.58E−08 |
| 17 | 2053 | 5.75E−08 | 1607 | 6.22E−08 | 2089 | 5.71E−08 |
| 18 | 2089 | 5.75E−08 | 357 | 6.69E−08 | 546 | 6.18E−08 |
| 19 | 546 | 6.38E−08 | 546 | 6.94E−08 | 1607 | 6.19E−08 |
| 20 | 357 | 6.92E−08 | 481 | 6.99E−08 | 481 | 6.73E−08 |
| 21 | 1607 | 6.96E−08 | 1198 | 8.00E−08 | 357 | 9.80E−08 |
| 22 | 481 | 7.08E−08 | 637 | 8.94E−08 | 155 | 9.86E−08 |
| 23 | 2321 | 8.25E−08 | 670 | 1.10E−07 | 685 | 1.96E−07 |
| 24 | 155 | 1.07E−07 | 155 | 1.13E−07 | 2232 | 1.89E−07 |
| 25 | 1065 | 1.71E−07 | 2053 | 1.74E−07 | 551 | 1.92E−07 |
| 26 | 551 | 1.85E−07 | 551 | 1.75E−07 | 2216 | 2.04E−07 |
| 27 | 2232 | 2.00E−07 | 893 | 1.84E−07 | 538 | 2.29E−07 |
| 28 | 2216 | 2.05E−07 | 1934 | 1.92E−07 | 663 | 2.34E−07 |
| 29 | 1117 | 2.29E−07 | 2232 | 1.97E−07 | 274 | 2.39E−07 |
| 30 | 538 | 2.31E−07 | 2216 | 2.12E−07 | 1 | 2.44E−07 |
| 31 | 1915 | 2.36E−07 | 274 | 2.28E−07 | 637 | 2.53E−07 |
| 32 | 1860 | 2.40E−07 | 538 | 2.34E−07 | 919 | 2.61E−07 |
| 33 | 663 | 2.61E−07 | 663 | 2.46E−07 | 2053 | 2.84E−07 |
| 34 | 637 | 3.01E−07 | 1556 | 2.89E−07 | 867 | 2.86E−07 |
| 35 | 2134 | 3.36E−07 | 1332 | 3.01E−07 | 1323 | 2.89E−07 |
| 36 | 393 | 3.79E−07 | 246 | 3.14E−07 | 486 | 2.94E−07 |
| 37 | 755 | 3.80E−07 | 1732 | 3.20E−07 | 1934 | 3.10E−07 |
| 38 | 1175 | 3.85E−07 | 1 | 3.30E−07 | 755 | 3.37E−07 |
| 39 | 1714 | 3.89E−07 | 755 | 3.43E−07 | 2134 | 3.37E−07 |
| 40 | 1732 | 3.90E−07 | 904 | 3.43E−07 | 890 | 3.63E−07 |
| 41 | 890 | 3.96E−07 | 890 | 3.56E−07 | 393 | 3.75E−07 |
| 42 | 486 | 4.06E−07 | 393 | 3.72E−07 | 1628 | 3.90E−07 |
| 43 | 586 | 4.09E−07 | 2134 | 3.86E−07 | 586 | 4.04E−07 |
| 44 | 649 | 4.11E−07 | 478 | 3.95E−07 | 1714 | 4.10E−07 |
| 45 | 1934 | 4.17E−07 | 1065 | 3.98E−07 | 478 | 4.19E−07 |
| 46 | 478 | 4.18E−07 | 1714 | 3.99E−07 | 491 | 4.43E−07 |
| 47 | 246 | 4.19E−07 | 550 | 4.02E−07 | 1915 | 4.43E−07 |
| 48 | 1628 | 4.23E−07 | 586 | 4.06E−07 | 282 | 4.57E−07 |
| 49 | 491 | 4.38E−07 | 1628 | 4.06E−07 | 1631 | 4.61E−07 |
| 50 | 282 | 4.57E−07 | 1175 | 4.10E−07 | 545 | 4.73E−07 |
| 51 | 545 | 4.59E−07 | 491 | 4.16E−07 | 818 | 4.93E−07 |
| 52 | 1631 | 4.62E−07 | 1323 | 4.28E−07 | 649 | 5.12E−07 |
| 53 | 492 | 4.88E−07 | 486 | 4.54E−07 | 429 | 5.14E−07 |
| 54 | 818 | 5.03E−07 | 282 | 4.57E−07 | 2221 | 5.16E−07 |
| 55 | 1323 | 5.08E−07 | 1631 | 4.61E−07 | 1099 | 5.18E−07 |
| 56 | 536 | 5.18E−07 | 545 | 4.66E−07 | 1860 | 5.23E−07 |
| 57 | 429 | 5.19E−07 | 818 | 4.80E−07 | 501 | 5.35E−07 |
| 58 | 1099 | 5.21E−07 | 485 | 4.96E−07 | 1917 | 5.39E−07 |
| 59 | 1917 | 5.39E−07 | 429 | 5.18E−07 | 2018 | 5.46E−07 |
| 60 | 2018 | 5.46E−07 | 1748 | 5.29E−07 | 719 | 5.50E−07 |
| 61 | 719 | 5.57E−07 | 867 | 5.34E−07 | 1748 | 5.50E−07 |
| 62 | 550 | 5.58E−07 | 1438 | 5.36E−07 | 1641 | 5.58E−07 |
| 63 | 1973 | 5.69E−07 | 1915 | 5.37E−07 | 1973 | 5.66E−07 |
| 64 | 1364 | 6.04E−07 | 1917 | 5.39E−07 | 1065 | 6.04E−07 |
| 65 | 501 | 6.14E−07 | 719 | 5.50E−07 | 566 | 6.19E−07 |
| 66 | 566 | 6.37E−07 | 2018 | 5.52E−07 | 1364 | 6.56E−07 |
| 67 | 503 | 6.44E−07 | 1099 | 5.57E−07 | 1438 | 6.67E−07 |
| 68 | 729 | 6.69E−07 | 1973 | 5.68E−07 | 2074 | 6.70E−07 |
| 69 | 636 | 6.85E−07 | 2262 | 5.80E−07 | 485 | 6.72E−07 |
| 70 | 485 | 6.90E−07 | 501 | 5.84E−07 | 1585 | 6.76E−07 |
| 71 | 593 | 6.91E−07 | 1364 | 6.36E−07 | 550 | 6.80E−07 |
| 72 | 867 | 6.97E−07 | 566 | 6.54E−07 | 636 | 6.82E−07 |
| 73 | 1 | 7.00E−07 | 2309 | 6.67E−07 | 2321 | 6.82E−07 |
| 74 | 702 | 7.04E−07 | 729 | 6.74E−07 | 729 | 6.93E−07 |
| 75 | 1075 | 7.05E−07 | 1075 | 6.94E−07 | 1075 | 6.98E−07 |
| 76 | 567 | 7.13E−07 | 492 | 7.08E−07 | 2309 | 6.99E−07 |
| 77 | 2309 | 7.25E−07 | 636 | 7.08E−07 | 593 | 7.00E−07 |
| 78 | 1748 | 7.50E−07 | 649 | 7.23E−07 | 536 | 7.07E−07 |
| 79 | 1692 | 7.55E−07 | 2321 | 7.29E−07 | 2262 | 7.30E−07 |
| 80 | 1013 | 8.04E−07 | 536 | 7.34E−07 | 567 | 7.32E−07 |
| 81 | 479 | 8.16E−07 | 593 | 7.34E−07 | 479 | 7.37E−07 |
| 82 | 502 | 8.17E−07 | 567 | 7.41E−07 | 1175 | 7.66E−07 |
| 83 | 1908 | 8.21E−07 | 702 | 7.43E−07 | 1777 | 7.67E−07 |
| 84 | 822 | 8.24E−07 | 2287 | 7.57E−07 | 1692 | 7.72E−07 |
| 85 | 1675 | 8.55E−07 | 483 | 7.58E−07 | 681 | 7.90E−07 |
| 86 | 1295 | 8.75E−07 | 502 | 7.58E−07 | 1013 | 7.92E−07 |
| 87 | 1009 | 8.88E−07 | 479 | 7.63E−07 | 2177 | 7.92E−07 |
| 88 | 1274 | 8.92E−07 | 1860 | 7.78E−07 | 483 | 8.42E−07 |
| 89 | 2287 | 9.03E−07 | 1692 | 7.92E−07 | 1675 | 8.55E−07 |
| 90 | 632 | 9.04E−07 | 1013 | 7.98E−07 | 1295 | 8.70E−07 |
| 91 | 904 | 9.07E−07 | 1675 | 8.48E−07 | 492 | 8.74E−07 |
| 92 | 1624 | 9.12E−07 | 632 | 8.79E−07 | 1624 | 8.74E−07 |
| 93 | 474 | 9.28E−07 | 1295 | 8.80E−07 | 502 | 8.82E−07 |
| 94 | 2259 | 9.32E−07 | 1274 | 8.82E−07 | 2259 | 8.95E−07 |
| 95 | 431 | 9.33E−07 | 759 | 9.05E−07 | 822 | 8.96E−07 |
| 96 | 238 | 9.39E−07 | 1009 | 9.09E−07 | 1009 | 8.97E−07 |
| 97 | 505 | 9.42E−07 | 474 | 9.30E−07 | 474 | 8.98E−07 |
| 98 | 1968 | 9.43E−07 | 1968 | 9.44E−07 | 632 | 8.99E−07 |
| 99 | 2262 | 9.50E−07 | 1908 | 9.52E−07 | 2058 | 9.07E−07 |
| 100 | 919 | 9.54E−07 | 1230 | 9.64E−07 | 1274 | 9.14E−07 |

TABLE 14
Predicted inhibitors of HIV capsid

| | |
|---|---|
| 1E6J C-terminal | 1E6J N-terminal |

| | | | | |
|---|---|---|---|---|
| Rank | Drug ID | Calculated Ki | Drug ID | Calculated Ki |

SEED 1

| | | | | |
|---|---|---|---|---|
| 1 | 553 | 7.84E−08 | 1951 | 2.55E−07 |
| 2 | 577 | 9.16E−08 | 2321 | 7.54E−07 |
| 3 | 548 | 1.03E−07 | 637 | 7.76E−07 |
| 4 | 552 | 1.53E−07 | 1672 | 1.00E−06 |
| 5 | 546 | 1.55E−07 | 1 | 1.67E−06 |
| 6 | 637 | 1.65E−07 | 553 | 1.90E−06 |
| 7 | 27 | 1.76E−07 | 591 | 2.06E−06 |
| 8 | 545 | 1.91E−07 | 164 | 2.10E−06 |
| 9 | 1176 | 2.14E−07 | 551 | 2.32E−06 |
| 10 | 681 | 2.25E−07 | 1176 | 2.33E−06 |
| 11 | 448 | 2.77E−07 | 1777 | 2.34E−06 |
| 12 | 550 | 3.04E−07 | 155 | 2.40E−06 |
| 13 | 36 | 3.46E−07 | 2322 | 2.44E−06 |
| 14 | 1460 | 3.46E−07 | 1332 | 2.76E−06 |
| 15 | 593 | 3.66E−07 | 1658 | 2.87E−06 |
| 16 | 818 | 3.97E−07 | 151 | 3.11E−06 |
| 17 | 1117 | 4.13E−07 | 1089 | 3.12E−06 |
| 18 | 2089 | 4.33E−07 | 2320 | 3.63E−06 |
| 19 | 164 | 4.65E−07 | 1323 | 3.68E−06 |
| 20 | 1607 | 4.94E−07 | 1611 | 3.69E−06 |
| 21 | 155 | 5.35E−07 | 393 | 3.72E−06 |
| 22 | 517 | 5.59E−07 | 1108 | 3.78E−06 |
| 23 | 2170 | 5.89E−07 | 2309 | 3.81E−06 |
| 24 | 1985 | 5.96E−07 | 491 | 3.87E−06 |
| 25 | 1099 | 6.28E−07 | 546 | 4.20E−06 |
| 26 | 2174 | 6.35E−07 | 2156 | 4.35E−06 |
| 27 | 551 | 6.40E−07 | 1989 | 4.65E−06 |
| 28 | 670 | 6.40E−07 | 2289 | 4.76E−06 |
| 29 | 647 | 7.22E−07 | 36 | 4.78E−06 |
| 30 | 1086 | 7.25E−07 | 1767 | 4.87E−06 |
| 31 | 501 | 7.33E−07 | 1666 | 4.92E−06 |
| 32 | 1628 | 7.58E−07 | 2091 | 5.00E−06 |
| 33 | 350 | 7.71E−07 | 738 | 5.10E−06 |
| 34 | 538 | 7.74E−07 | 521 | 5.11E−06 |
| 35 | 1562 | 7.88E−07 | 628 | 5.23E−06 |
| 36 | 131 | 7.99E−07 | 1939 | 5.48E−06 |
| 37 | 521 | 8.29E−07 | 2303 | 5.49E−06 |
| 38 | 549 | 8.44E−07 | 1837 | 5.70E−06 |
| 39 | 2155 | 8.50E−07 | 550 | 5.81E−06 |
| 40 | 1009 | 9.48E−07 | 545 | 5.82E−06 |
| 41 | 274 | 9.63E−07 | 1908 | 6.05E−06 |
| 42 | 626 | 9.95E−07 | 115 | 6.38E−06 |
| 43 | 284 | 1.04E−06 | 1711 | 6.43E−06 |
| 44 | 2009 | 1.04E−06 | 755 | 6.64E−06 |
| 45 | 632 | 1.06E−06 | 436 | 6.68E−06 |
| 46 | 1777 | 1.11E−06 | 577 | 6.73E−06 |
| 47 | 1748 | 1.14E−06 | 1431 | 6.78E−06 |
| 48 | 1991 | 1.16E−06 | 535 | 6.82E−06 |
| 49 | 1013 | 1.19E−06 | 1295 | 7.00E−06 |
| 50 | 7 | 1.26E−06 | 1924 | 7.06E−06 |
| 51 | 567 | 1.28E−06 | 552 | 7.25E−06 |
| 52 | 562 | 1.29E−06 | 2216 | 7.28E−06 |
| 53 | 357 | 1.36E−06 | 548 | 7.32E−06 |
| 54 | 1306 | 1.38E−06 | 1460 | 7.53E−06 |
| 55 | 1462 | 1.43E−06 | 576 | 7.54E−06 |
| 56 | 544 | 1.45E−06 | 1607 | 7.58E−06 |
| 57 | 1714 | 1.45E−06 | 485 | 7.67E−06 |
| 58 | 2286 | 1.45E−06 | 1151 | 7.68E−06 |
| 59 | 573 | 1.46E−06 | 488 | 7.79E−06 |
| 60 | 1603 | 1.46E−06 | 1985 | 7.89E−06 |
| 61 | 1598 | 1.47E−06 | 1639 | 7.93E−06 |
| 62 | 2138 | 1.49E−06 | 617 | 8.24E−06 |
| 63 | 1089 | 1.56E−06 | 593 | 8.54E−06 |
| 64 | 1090 | 1.63E−06 | 274 | 8.57E−06 |
| 65 | 1361 | 1.63E−06 | 464 | 8.63E−06 |
| 66 | 1585 | 1.65E−06 | 1462 | 8.96E−06 |
| 67 | 643 | 1.66E−06 | 1872 | 9.01E−06 |
| 68 | 1420 | 1.67E−06 | 350 | 9.09E−06 |
| 69 | 1450 | 1.67E−06 | 2219 | 9.48E−06 |
| 70 | 725 | 1.68E−06 | 282 | 9.64E−06 |
| 71 | 554 | 1.73E−06 | 357 | 9.84E−06 |
| 72 | 42 | 1.75E−06 | 2181 | 1.04E−05 |
| 73 | 1312 | 1.75E−06 | 17 | 1.09E−05 |
| 74 | 1703 | 1.75E−06 | 1135 | 1.09E−05 |
| 75 | 1075 | 1.76E−06 | 334 | 1.11E−05 |
| 76 | 561 | 1.79E−06 | 871 | 1.12E−05 |
| 77 | 616 | 1.79E−06 | 1973 | 1.12E−05 |
| 78 | 547 | 1.80E−06 | 3 | 1.13E−05 |
| 79 | 845 | 1.80E−06 | 502 | 1.14E−05 |
| 80 | 2342 | 1.81E−06 | 2155 | 1.15E−05 |
| 81 | 503 | 1.86E−06 | 880 | 1.17E−05 |
| 82 | 2214 | 1.86E−06 | 1938 | 1.17E−05 |
| 83 | 2549 | 1.89E−06 | 818 | 1.18E−05 |
| 84 | 566 | 1.96E−06 | 2009 | 1.21E−05 |
| 85 | 661 | 1.99E−06 | 1287 | 1.23E−05 |
| 86 | 1593 | 2.00E−06 | 520 | 1.24E−05 |
| 87 | 2134 | 2.01E−06 | 411 | 1.25E−05 |
| 88 | 944 | 2.04E−06 | 238 | 1.26E−05 |
| 89 | 615 | 2.05E−06 | 480 | 1.27E−05 |
| 90 | 151 | 2.12E−06 | 1712 | 1.29E−05 |
| 91 | 331 | 2.15E−06 | 567 | 1.31E−05 |
| 92 | 972 | 2.15E−06 | 472 | 1.32E−05 |
| 93 | 882 | 2.17E−06 | 1834 | 1.33E−05 |
| 94 | 2250 | 2.19E−06 | 1921 | 1.35E−05 |
| 95 | 1639 | 2.20E−06 | 566 | 1.37E−05 |
| 96 | 1666 | 2.32E−06 | 1129 | 1.37E−05 |
| 97 | 446 | 2.35E−06 | 1597 | 1.39E−05 |
| 98 | 880 | 2.38E−06 | 1640 | 1.40E−05 |
| 99 | 872 | 2.48E−06 | 681 | 1.42E−05 |
| 100 | 202 | 2.50E−06 | 1436 | 1.43E−05 |

SEED 2

| | | | | |
|---|---|---|---|---|
| 1 | 553 | 7.74E−08 | 2321 | 7.29E−07 |
| 2 | 577 | 9.29E−08 | 1332 | 9.24E−07 |
| 3 | 1921 | 9.55E−08 | 637 | 9.29E−07 |
| 4 | 548 | 1.07E−07 | 1089 | 9.54E−07 |
| 5 | 552 | 1.47E−07 | 1951 | 1.31E−06 |
| 6 | 546 | 1.53E−07 | 1 | 1.48E−06 |
| 7 | 27 | 1.67E−07 | 553 | 1.99E−06 |
| 8 | 637 | 1.71E−07 | 591 | 2.07E−06 |
| 9 | 545 | 1.75E−07 | 274 | 2.17E−06 |
| 10 | 1176 | 1.99E−07 | 2320 | 2.23E−06 |
| 11 | 1908 | 2.20E−07 | 155 | 2.30E−06 |
| 12 | 550 | 2.50E−07 | 1176 | 2.32E−06 |
| 13 | 681 | 2.58E−07 | 551 | 2.33E−06 |
| 14 | 448 | 2.89E−07 | 2322 | 2.59E−06 |
| 15 | 1460 | 3.30E−07 | 485 | 3.05E−06 |
| 16 | 36 | 3.44E−07 | 151 | 3.18E−06 |
| 17 | 1117 | 3.45E−07 | 36 | 3.28E−06 |
| 18 | 593 | 3.53E−07 | 1357 | 3.31E−06 |
| 19 | 818 | 3.89E−07 | 164 | 3.58E−06 |
| 20 | 2089 | 4.10E−07 | 393 | 3.72E−06 |
| 21 | 1837 | 4.71E−07 | 1108 | 3.79E−06 |
| 22 | 2009 | 4.74E−07 | 1611 | 3.87E−06 |
| 23 | 1607 | 4.99E−07 | 2309 | 3.91E−06 |
| 24 | 164 | 5.48E−07 | 1767 | 4.35E−06 |
| 25 | 517 | 5.57E−07 | 546 | 4.45E−06 |
| 26 | 551 | 5.62E−07 | 3 | 4.73E−06 |
| 27 | 2170 | 5.84E−07 | 2091 | 4.95E−06 |
| 28 | 1985 | 6.02E−07 | 521 | 5.07E−06 |
| 29 | 1086 | 6.19E−07 | 628 | 5.26E−06 |
| 30 | 1099 | 6.22E−07 | 550 | 5.37E−06 |
| 31 | 155 | 6.23E−07 | 1323 | 5.50E−06 |
| 32 | 1777 | 7.16E−07 | 2303 | 5.50E−06 |
| 33 | 647 | 7.22E−07 | 567 | 5.57E−06 |
| 34 | 1628 | 7.30E−07 | 1939 | 5.60E−06 |
| 35 | 350 | 7.66E−07 | 1985 | 5.65E−06 |
| 36 | 2155 | 7.75E−07 | 2039 | 5.67E−06 |
| 37 | 538 | 7.85E−07 | 545 | 5.76E−06 |
| 38 | 449 | 7.87E−07 | 2156 | 5.85E−06 |
| 39 | 1968 | 7.93E−07 | 1013 | 6.16E−06 |
| 40 | 2174 | 8.10E−07 | 535 | 6.20E−06 |
| 41 | 131 | 8.18E−07 | 1989 | 6.30E−06 |
| 42 | 501 | 8.41E−07 | 755 | 6.39E−06 |
| 43 | 1469 | 8.42E−07 | 115 | 6.40E−06 |
| 44 | 549 | 8.44E−07 | 1607 | 6.41E−06 |
| 45 | 521 | 8.54E−07 | 1018 | 6.66E−06 |
| 46 | 1009 | 8.85E−07 | 1837 | 6.81E−06 |
| 47 | 1951 | 9.24E−07 | 593 | 6.95E−06 |
| 48 | 626 | 1.01E−06 | 1431 | 6.95E−06 |
| 49 | 632 | 1.04E−06 | 1295 | 6.96E−06 |
| 50 | 1973 | 1.04E−06 | 1924 | 7.00E−06 |
| 51 | 1462 | 1.05E−06 | 1711 | 7.10E−06 |
| 52 | 284 | 1.06E−06 | 577 | 7.24E−06 |
| 53 | 436 | 1.08E−06 | 552 | 7.27E−06 |
| 54 | 1910 | 1.08E−06 | 576 | 7.48E−06 |
| 55 | 1945 | 1.08E−06 | 1460 | 7.50E−06 |
| 56 | 1013 | 1.09E−06 | 617 | 7.66E−06 |
| 57 | 1939 | 1.12E−06 | 681 | 8.02E−06 |
| 58 | 877 | 1.16E−06 | 880 | 8.32E−06 |
| 59 | 1991 | 1.16E−06 | 548 | 8.44E−06 |
| 60 | 2134 | 1.21E−06 | 1117 | 8.49E−06 |
| 61 | 7 | 1.25E−06 | 350 | 8.61E−06 |
| 62 | 42 | 1.28E−06 | 1151 | 8.92E−06 |
| 63 | 562 | 1.29E−06 | 1872 | 8.97E−06 |
| 64 | 357 | 1.30E−06 | 2089 | 9.21E−06 |
| 65 | 1090 | 1.31E−06 | 2219 | 9.49E−06 |
| 66 | 1960 | 1.31E−06 | 579 | 9.53E−06 |
| 67 | 1306 | 1.35E−06 | 282 | 9.64E−06 |
| 68 | 1714 | 1.38E−06 | 1287 | 9.66E−06 |
| 69 | 566 | 1.41E−06 | 357 | 9.77E−06 |
| 70 | 1361 | 1.46E−06 | 1777 | 9.91E−06 |
| 71 | 1598 | 1.46E−06 | 2216 | 1.02E−05 |
| 72 | 567 | 1.51E−06 | 436 | 1.05E−05 |
| 73 | 544 | 1.54E−06 | 1191 | 1.05E−05 |
| 74 | 2286 | 1.54E−06 | 2009 | 1.05E−05 |
| 75 | 573 | 1.57E−06 | 1233 | 1.06E−05 |
| 76 | 643 | 1.59E−06 | 2181 | 1.06E−05 |
| 77 | 1603 | 1.59E−06 | 17 | 1.07E−05 |
| 78 | 1420 | 1.66E−06 | 1973 | 1.07E−05 |
| 79 | 1450 | 1.68E−06 | 449 | 1.09E−05 |
| 80 | 1748 | 1.71E−06 | 1135 | 1.09E−05 |
| 81 | 554 | 1.73E−06 | 871 | 1.12E−05 |
| 82 | 1312 | 1.73E−06 | 334 | 1.14E−05 |
| 83 | 1075 | 1.74E−06 | 835 | 1.14E−05 |
| 84 | 547 | 1.78E−06 | 1129 | 1.15E−05 |
| 85 | 561 | 1.79E−06 | 818 | 1.16E−05 |
| 86 | 616 | 1.79E−06 | 411 | 1.17E−05 |
| 87 | 1703 | 1.80E−06 | 488 | 1.17E−05 |
| 88 | 2342 | 1.83E−06 | 1938 | 1.17E−05 |
| 89 | 1585 | 1.87E−06 | 238 | 1.27E−05 |
| 90 | 2214 | 1.89E−06 | 2289 | 1.28E−05 |
| 91 | 2549 | 1.90E−06 | 1921 | 1.29E−05 |
| 92 | 1323 | 1.92E−06 | 2286 | 1.31E−05 |
| 93 | 944 | 1.95E−06 | 566 | 1.32E−05 |
| 94 | 661 | 1.96E−06 | 472 | 1.34E−05 |
| 95 | 1834 | 1.97E−06 | 1834 | 1.40E−05 |
| 96 | 1593 | 2.00E−06 | 1597 | 1.42E−05 |
| 97 | 822 | 2.07E−06 | 480 | 1.43E−05 |
| 98 | 1562 | 2.09E−06 | 1712 | 1.43E−05 |
| 99 | 882 | 2.11E−06 | 462 | 1.44E−05 |
| 100 | 151 | 2.12E−06 | 1141 | 1.44E−05 |

SEED 3

| | | | | |
|---|---|---|---|---|
| 1 | 553 | 7.53E−08 | 2321 | 7.69E−07 |
| 2 | 577 | 9.10E−08 | 1672 | 1.01E−06 |
| 3 | 546 | 1.45E−07 | 1951 | 1.12E−06 |
| 4 | 552 | 1.52E−07 | 637 | 1.19E−06 |
| 5 | 27 | 1.59E−07 | 485 | 1.69E−06 |
| 6 | 1834 | 1.77E−07 | 164 | 1.77E−06 |
| 7 | 1908 | 1.80E−07 | 553 | 1.89E−06 |
| 8 | 545 | 1.84E−07 | 1176 | 2.03E−06 |
| 9 | 1176 | 1.99E−07 | 591 | 2.07E−06 |
| 10 | 1117 | 2.12E−07 | 1323 | 2.21E−06 |
| 11 | 1921 | 2.42E−07 | 155 | 2.23E−06 |
| 12 | 550 | 2.54E−07 | 1 | 2.27E−06 |
| 13 | 637 | 2.56E−07 | 1332 | 2.33E−06 |
| 14 | 448 | 2.89E−07 | 551 | 2.36E−06 |
| 15 | 1460 | 3.25E−07 | 274 | 2.41E−06 |
| 16 | 36 | 3.49E−07 | 1658 | 2.45E−06 |
| 17 | 548 | 3.50E−07 | 2322 | 2.59E−06 |
| 18 | 593 | 3.73E−07 | 2320 | 2.66E−06 |
| 19 | 818 | 3.98E−07 | 151 | 3.05E−06 |
| 20 | 2089 | 4.17E−07 | 2216 | 3.25E−06 |
| 21 | 1607 | 4.60E−07 | 1108 | 3.71E−06 |
| 22 | 1837 | 4.72E−07 | 393 | 3.75E−06 |
| 23 | 164 | 5.30E−07 | 2309 | 3.84E−06 |
| 24 | 2174 | 5.41E−07 | 1611 | 3.88E−06 |
| 25 | 155 | 5.61E−07 | 1680 | 3.96E−06 |
| 26 | 517 | 5.62E−07 | 546 | 4.42E−06 |
| 27 | 2170 | 5.84E−07 | 1767 | 4.53E−06 |
| 28 | 1985 | 5.99E−07 | 2156 | 4.73E−06 |
| 29 | 1099 | 6.12E−07 | 36 | 4.83E−06 |
| 30 | 551 | 6.40E−07 | 2091 | 4.95E−06 |
| 31 | 681 | 6.40E−07 | 1666 | 4.96E−06 |
| 32 | 501 | 7.08E−07 | 521 | 5.10E−06 |
| 33 | 647 | 7.22E−07 | 1989 | 5.18E−06 |
| 34 | 1086 | 7.23E−07 | 628 | 5.29E−06 |
| 35 | 1628 | 7.28E−07 | 2303 | 5.50E−06 |
| 36 | 2009 | 7.29E−07 | 1939 | 5.57E−06 |
| 37 | 350 | 7.55E−07 | 545 | 5.64E−06 |
| 38 | 1968 | 7.84E−07 | 1837 | 5.65E−06 |
| 39 | 1777 | 7.86E−07 | 550 | 5.84E−06 |
| 40 | 131 | 7.97E−07 | 115 | 6.03E−06 |
| 41 | 2155 | 7.98E−07 | 1287 | 6.11E−06 |
| 42 | 1009 | 8.43E−07 | 1013 | 6.20E−06 |
| 43 | 549 | 8.46E−07 | 2039 | 6.22E−06 |
| 44 | 521 | 8.66E−07 | 1607 | 6.49E−06 |
| 45 | 538 | 8.98E−07 | 755 | 6.59E−06 |
| 46 | 1013 | 9.40E−07 | 535 | 6.71E−06 |
| 47 | 626 | 1.02E−06 | 593 | 6.74E−06 |
| 48 | 632 | 1.03E−06 | 577 | 6.85E−06 |
| 49 | 1910 | 1.03E−06 | 1295 | 6.99E−06 |
| 50 | 1973 | 1.04E−06 | 1924 | 6.99E−06 |
| 51 | 284 | 1.06E−06 | 1711 | 7.04E−06 |
| 52 | 1939 | 1.12E−06 | 1431 | 7.10E−06 |
| 53 | 1991 | 1.16E−06 | 552 | 7.13E−06 |
| 54 | 1945 | 1.20E−06 | 1018 | 7.16E−06 |
| 55 | 357 | 1.23E−06 | 576 | 7.45E−06 |
| 56 | 1469 | 1.23E−06 | 488 | 7.52E−06 |
| 57 | 236 | 1.24E−06 | 1460 | 7.57E−06 |
| 58 | 7 | 1.26E−06 | 511 | 8.02E−06 |
| 59 | 1960 | 1.31E−06 | 1639 | 8.06E−06 |
| 60 | 562 | 1.38E−06 | 1985 | 8.20E−06 |
| 61 | 1714 | 1.39E−06 | 617 | 8.32E−06 |
| 62 | 42 | 1.41E−06 | 436 | 8.54E−06 |
| 63 | 544 | 1.42E−06 | 2089 | 8.62E−06 |
| 64 | 2286 | 1.46E−06 | 1151 | 8.92E−06 |
| 65 | 1090 | 1.49E−06 | 548 | 9.12E−06 |
| 66 | 566 | 1.50E−06 | 1462 | 9.14E−06 |
| 67 | 1361 | 1.50E−06 | 2219 | 9.50E−06 |
| 68 | 1603 | 1.53E−06 | 2181 | 9.86E−06 |
| 69 | 573 | 1.56E−06 | 357 | 1.00E−05 |
| 70 | 643 | 1.63E−06 | 282 | 1.02E−05 |
| 71 | 561 | 1.68E−06 | 3 | 1.06E−05 |
| 72 | 1450 | 1.70E−06 | 472 | 1.07E−05 |
| 73 | 1703 | 1.71E−06 | 738 | 1.08E−05 |
| 74 | 554 | 1.73E−06 | 1135 | 1.09E−05 |
| 75 | 1075 | 1.74E−06 | 17 | 1.11E−05 |
| 76 | 2549 | 1.77E−06 | 1357 | 1.11E−05 |
| 77 | 1312 | 1.78E−06 | 871 | 1.12E−05 |
| 78 | 547 | 1.80E−06 | 334 | 1.13E−05 |
| 79 | 616 | 1.81E−06 | 1436 | 1.14E−05 |
| 80 | 2342 | 1.82E−06 | 1921 | 1.15E−05 |
| 81 | 2134 | 1.87E−06 | 818 | 1.17E−05 |
| 82 | 2214 | 1.87E−06 | 1938 | 1.17E−05 |
| 83 | 670 | 1.95E−06 | 1089 | 1.21E−05 |
| 84 | 944 | 1.98E−06 | 1973 | 1.22E−05 |
| 85 | 1462 | 1.98E−06 | 411 | 1.23E−05 |
| 86 | 274 | 2.00E−06 | 2155 | 1.25E−05 |
| 87 | 386 | 2.03E−06 | 1640 | 1.26E−05 |
| 88 | 2262 | 2.03E−06 | 567 | 1.28E−05 |
| 89 | 2154 | 2.04E−06 | 1777 | 1.31E−05 |
| 90 | 615 | 2.05E−06 | 1117 | 1.32E−05 |
| 91 | 661 | 2.05E−06 | 1304 | 1.32E−05 |
| 92 | 1357 | 2.05E−06 | 464 | 1.35E−05 |
| 93 | 567 | 2.09E−06 | 566 | 1.35E−05 |
| 94 | 151 | 2.12E−06 | 480 | 1.38E−05 |
| 95 | 882 | 2.14E−06 | 626 | 1.38E−05 |
| 96 | 331 | 2.16E−06 | 2286 | 1.40E−05 |
| 97 | 972 | 2.20E−06 | 1597 | 1.41E−05 |
| 98 | 2250 | 2.21E−06 | 238 | 1.42E−05 |
| 99 | 449 | 2.27E−06 | 1070 | 1.42E−05 |
| 100 | 1631 | 2.40E−06 | 462 | 1.45E−05 |

TABLE 15
Predicted inhibitors of Mycobaterium tuberculosis [symbol]
EED 1

| | 1F61 | | 1GR0 | | 1IDS | | 1N2E | | 1N8W | |
|---|---|---|---|---|---|---|---|---|---|---|
| [symbol] ank | Drug ID | Calculated Ki | Drug ID | Calculated Ki | Drug ID | Calculated Ki | Drug ID | Calculated Ki | Drug ID | Calculated Ki |
| 1 | 1921 | 2.83E−13 | 2303 | 9.39E−15 | 1647 | 6.30E−10 | 1012 | 1.39E−10 | 2303 | 5.22E−10 |
| 2 | 1462 | 7.13E−13 | 893 | 7.50E−12 | 1584 | 2.40E−07 | 2330 | 1.68E−10 | 1117 | 8.30E−09 |
| 3 | 2303 | 1.16E−12 | 1559 | 1.31E−11 | 1332 | 2.88E−07 | 275 | 2.63E−10 | 2287 | 9.17E−09 |
| 4 | 1570 | 3.56E−12 | 2287 | 1.65E−11 | 1921 | 4.99E−07 | 499 | 2.97E−10 | 1921 | 1.47E−08 |
| 5 | 1565 | 3.92E−12 | 685 | 2.91E−11 | 550 | 5.18E−07 | 1777 | 3.17E−10 | 637 | 1.58E−08 |
| 6 | 1469 | 4.31E−12 | 919 | 4.53E−11 | 1909 | 6.82E−07 | 560 | 3.21E−10 | 499 | 2.04E−08 |
| 7 | 685 | 9.07E−12 | 1117 | 5.87E−11 | 2174 | 7.12E−07 | 274 | 3.53E−10 | 1973 | 2.30E−08 |
| 8 | 2262 | 1.98E−11 | 492 | 6.38E−11 | 2216 | 9.93E−07 | 1921 | 3.56E−10 | 2125 | 2.59E−08 |
| 9 | 1607 | 7.50E−11 | 1921 | 6.67E−11 | 2289 | 1.38E−06 | 551 | 6.06E−10 | 550 | 2.78E−08 |
| 10 | 1364 | 8.26E−11 | 548 | 6.88E−11 | 1721 | 1.87E−06 | 1680 | 6.44E−10 | 155 | 3.04E−08 |
| 11 | 546 | 8.35E−11 | 1951 | 1.04E−10 | 551 | 1.99E−06 | 472 | 8.02E−10 | 551 | 3.30E−08 |
| 12 | 2287 | 1.15E−10 | 2174 | 1.29E−10 | 472 | 2.01E−06 | 2321 | 8.86E−10 | 2018 | 3.44E−08 |
| 13 | 1065 | 1.43E−10 | 1565 | 1.51E−10 | 36 | 2.03E−06 | 1780 | 1.06E−09 | 548 | 3.65E−08 |
| 14 | 904 | 1.86E−10 | 553 | 1.92E−10 | 1767 | 2.18E−06 | 236 | 1.45E−09 | 2216 | 3.67E−08 |
| 15 | 893 | 1.92E−10 | 245 | 1.99E−10 | 1309 | 2.22E−06 | 1951 | 1.46E−09 | 1680 | 5.42E−08 |
| 16 | 2330 | 2.18E−10 | 552 | 2.10E−10 | 552 | 2.32E−06 | 2216 | 1.78E−09 | 2058 | 5.53E−08 |
| 17 | 919 | 2.46E−10 | 1915 | 2.17E−10 | 545 | 2.52E−06 | 545 | 1.98E−09 | 492 | 5.56E−08 |
| 18 | 553 | 2.56E−10 | 1680 | 2.37E−10 | 1 | 2.59E−06 | 1666 | 2.34E−09 | 553 | 5.96E−08 |
| 19 | 637 | 3.20E−10 | 1138 | 3.17E−10 | 637 | 2.75E−06 | 2289 | 2.37E−09 | 350 | 6.19E−08 |
| 20 | 2174 | 4.26E−10 | 1198 | 3.23E−10 | 449 | 2.86E−06 | 318 | 3.07E−09 | 1607 | 6.30E−08 |
| 21 | 877 | 4.28E−10 | 2300 | 3.42E−10 | 71 | 2.96E−06 | 205 | 3.15E−09 | 2322 | 6.53E−08 |
| 22 | 1117 | 4.28E−10 | 2321 | 3.95E−10 | 1839 | 2.98E−06 | 1762 | 3.29E−09 | 2321 | 6.87E−08 |
| 23 | 490 | 5.55E−10 | 488 | 3.98E−10 | 1968 | 3.08E−06 | 2329 | 3.36E−09 | 2300 | 6.91E−08 |
| 24 | 552 | 7.15E−10 | 482 | 4.05E−10 | 553 | 3.53E−06 | 479 | 3.52E−09 | 603 | 7.35E−08 |
| 25 | 2216 | 8.52E−10 | 554 | 5.19E−10 | 9 | 3.56E−06 | 1920 | 3.64E−09 | 17 | 7.44E−08 |
| 26 | 670 | 8.58E−10 | 2328 | 5.29E−10 | 533 | 3.62E−06 | 2171 | 3.69E−09 | 439 | 7.60E−08 |
| 27 | 1934 | 1.13E−09 | 1065 | 5.48E−10 | 482 | 3.78E−06 | 1585 | 3.84E−09 | 1985 | 8.26E−08 |
| 28 | 491 | 1.31E−09 | 491 | 6.00E−10 | 278 | 3.80E−06 | 553 | 3.95E−09 | 245 | 8.68E−08 |
| 29 | 2053 | 1.43E−09 | 2216 | 6.56E−10 | 546 | 3.87E−06 | 867 | 4.16E−09 | 2289 | 8.71E−08 |
| 30 | 1951 | 1.45E−09 | 551 | 7.05E−10 | 566 | 4.13E−06 | 2301 | 4.46E−09 | 2301 | 9.03E−08 |
| 31 | 399 | 1.52E−09 | 670 | 7.08E−10 | 1437 | 4.14E−06 | 1569 | 4.62E−09 | 2174 | 9.52E−08 |
| 32 | 2259 | 1.54E−09 | 2123 | 7.74E−10 | 334 | 4.34E−06 | 663 | 4.68E−09 | 1951 | 9.80E−08 |
| 33 | 2295 | 1.86E−09 | 1989 | 8.51E−10 | 2308 | 4.36E−06 | 637 | 5.25E−09 | 1989 | 9.92E−08 |
| 34 | 232 | 1.89E−09 | 550 | 8.99E−10 | 1716 | 4.42E−06 | 993 | 5.32E−09 | 472 | 1.02E−07 |
| 35 | 2009 | 2.00E−09 | 603 | 9.39E−10 | 618 | 4.46E−06 | 1973 | 5.34E−09 | 521 | 1.02E−07 |
| 36 | 1198 | 2.03E−09 | 490 | 1.06E−09 | 502 | 4.56E−06 | 1176 | 5.46E−09 | 893 | 1.04E−07 |
| 37 | 1438 | 2.18E−09 | 1438 | 1.07E−09 | 2134 | 4.60E−06 | 679 | 6.04E−09 | 501 | 1.10E−07 |
| 38 | 379 | 2.19E−09 | 1732 | 1.12E−09 | 2320 | 4.62E−06 | 566 | 6.08E−09 | 1462 | 1.11E−07 |
| 39 | 482 | 2.96E−09 | 478 | 1.20E−09 | 440 | 4.69E−06 | 1512 | 6.19E−09 | 271 | 1.16E−07 |
| 40 | 2018 | 3.02E−09 | 1462 | 1.23E−09 | 2155 | 4.99E−06 | 2288 | 6.33E−09 | 736 | 1.41E−07 |
| 41 | 1561 | 3.10E−09 | 1973 | 1.36E−09 | 1939 | 5.20E−06 | 800 | 6.48E−09 | 670 | 1.46E−07 |
| 42 | 548 | 3.78E−09 | 127 | 1.39E−09 | 1783 | 5.24E−06 | 494 | 6.66E−09 | 2134 | 1.69E−07 |
| 43 | 2171 | 4.01E−09 | 489 | 1.39E−09 | 1663 | 5.57E−06 | 554 | 6.78E−09 | 554 | 1.72E−07 |
| 44 | 1323 | 4.67E−09 | 1469 | 1.44E−09 | 348 | 5.68E−06 | 759 | 6.82E−09 | 1332 | 1.73E−07 |
| 45 | 1631 | 4.94E−09 | 2074 | 1.45E−09 | 548 | 5.73E−06 | 436 | 6.89E−09 | 759 | 1.78E−07 |
| 46 | 1609 | 5.26E−09 | 485 | 1.46E−09 | 993 | 5.76E−06 | 1603 | 6.89E−09 | 1666 | 1.79E−07 |
| 47 | 1860 | 5.48E−09 | 271 | 1.54E−09 | 590 | 5.96E−06 | 473 | 6.94E−09 | 574 | 1.82E−07 |
| 48 | 1433 | 5.63E−09 | 545 | 1.64E−09 | 1807 | 5.97E−06 | 1418 | 7.13E−09 | 164 | 1.83E−07 |
| 49 | 1708 | 6.03E−09 | 1521 | 1.64E−09 | 535 | 6.02E−06 | 208 | 7.31E−09 | 482 | 1.84E−07 |
| 50 | 1915 | 6.85E−09 | 2330 | 1.65E−09 | 1140 | 6.03E−06 | 573 | 7.57E−09 | 567 | 1.93E−07 |
| 51 | 1246 | 6.94E−09 | 232 | 1.69E−09 | 755 | 6.04E−06 | 548 | 7.59E−09 | 613 | 1.95E−07 |
| 52 | 1552 | 7.39E−09 | 1287 | 1.80E−09 | 2284 | 6.08E−06 | 711 | 7.72E−09 | 328 | 1.99E−07 |
| 53 | 238 | 7.61E−09 | 2329 | 1.81E−09 | 488 | 6.24E−06 | 2156 | 7.90E−09 | 593 | 1.99E−07 |
| 54 | 2046 | 7.91E−09 | 637 | 1.82E−09 | 1634 | 6.43E−06 | 893 | 8.03E−09 | 545 | 2.01E−07 |
| 55 | 155 | 8.27E−09 | 493 | 1.91E−09 | 822 | 6.56E−06 | 310 | 8.28E−09 | 1837 | 2.04E−07 |
| 56 | 618 | 8.61E−09 | 2343 | 1.94E−09 | 567 | 6.64E−06 | 2174 | 8.31E−09 | 546 | 2.09E−07 |
| 57 | 1972 | 8.98E−09 | 2288 | 2.01E−09 | 1237 | 6.69E−06 | 448 | 8.53E−09 | 1672 | 2.10E−07 |
| 58 | 551 | 9.64E−09 | 494 | 2.15E−09 | 506 | 6.94E−06 | 2303 | 8.53E−09 | 552 | 2.11E−07 |
| 59 | 2289 | 9.90E−09 | 2239 | 2.23E−09 | 818 | 7.00E−06 | 1395 | 8.76E−09 | 2288 | 2.20E−07 |
| 60 | 449 | 1.21E−08 | 2289 | 2.23E−09 | 1960 | 7.12E−06 | 1831 | 8.84E−09 | 1938 | 2.52E−07 |
| 61 | 702 | 1.27E−08 | 591 | 2.41E−09 | 621 | 7.15E−06 | 164 | 1.08E−08 | 586 | 2.55E−07 |
| 62 | 431 | 1.29E−08 | 1570 | 2.46E−09 | 2078 | 7.30E−06 | 1390 | 1.08E−08 | 1 | 2.70E−07 |
| 63 | 27 | 1.32E−08 | 2009 | 2.68E−09 | 563 | 7.35E−06 | 552 | 1.15E−08 | 274 | 2.73E−07 |
| 64 | 2321 | 1.34E−08 | 2089 | 2.68E−09 | 1067 | 7.48E−06 | 2125 | 1.18E−08 | 822 | 2.83E−07 |
| 65 | 1866 | 1.35E−08 | 439 | 2.89E−09 | 736 | 7.50E−06 | 1497 | 1.19E−08 | 867 | 2.85E−07 |
| 66 | 2288 | 1.41E−08 | 2134 | 3.08E−09 | 1525 | 7.60E−06 | 2154 | 1.20E−08 | 449 | 2.89E−07 |
| 67 | 2074 | 1.42E−08 | 1968 | 3.32E−09 | 1985 | 7.70E−06 | 271 | 1.21E−08 | 2308 | 2.91E−07 |
| 68 | 1 | 1.45E−08 | 1641 | 3.43E−09 | 549 | 7.80E−06 | 550 | 1.21E−08 | 1967 | 2.94E−07 |
| 69 | 1680 | 1.47E−08 | 2232 | 3.49E−09 | 628 | 7.85E−06 | 1099 | 1.22E−08 | 445 | 3.03E−07 |
| 70 | 1658 | 1.48E−08 | 318 | 3.61E−09 | 1973 | 8.18E−06 | 2277 | 1.33E−08 | 1437 | 3.05E−07 |
| 71 | 2177 | 1.62E−08 | 546 | 3.63E−09 | 450 | 8.25E−06 | 1321 | 1.34E−08 | 1748 | 3.05E−07 |
| 72 | 2215 | 1.63E−08 | 481 | 3.69E−09 | 17 | 8.55E−06 | 2239 | 1.38E−08 | 115 | 3.06E−07 |
| 73 | 873 | 1.70E−08 | 505 | 3.82E−09 | 547 | 8.77E−06 | 603 | 1.40E−08 | 1476 | 3.06E−07 |
| 74 | 504 | 1.71E−08 | 2156 | 3.96E−09 | 1823 | 8.89E−06 | 702 | 1.41E−08 | 566 | 3.12E−07 |
| 75 | 939 | 1.80E−08 | 2332 | 4.13E−09 | 605 | 8.90E−06 | 1647 | 1.42E−08 | 939 | 3.21E−07 |
| 76 | 617 | 1.82E−08 | 238 | 4.24E−09 | 155 | 8.95E−06 | 1634 | 1.43E−08 | 1774 | 3.22E−07 |
| 77 | 496 | 1.86E−08 | 346 | 4.40E−09 | 2309 | 9.11E−06 | 2055 | 1.46E−08 | 1418 | 3.26E−07 |
| 78 | 205 | 1.93E−08 | 1 | 4.57E−09 | 984 | 9.27E−06 | 944 | 1.48E−08 | 2155 | 3.28E−07 |
| 79 | 1556 | 2.05E−08 | 604 | 4.64E−09 | 1611 | 9.49E−06 | 2215 | 1.53E−08 | 544 | 3.53E−07 |
| 80 | 488 | 2.11E−08 | 2018 | 4.65E−09 | 1453 | 9.54E−06 | 1108 | 1.54E−08 | 2152 | 3.53E−07 |
| 81 | 1620 | 2.12E−08 | 880 | 4.67E−09 | 1748 | 9.76E−06 | 1908 | 1.54E−08 | 882 | 3.54E−07 |
| 82 | 2328 | 2.16E−08 | 350 | 4.86E−09 | 1834 | 9.82E−06 | 247 | 1.55E−08 | 429 | 3.65E−07 |
| 83 | 350 | 2.18E−08 | 2125 | 4.92E−09 | 481 | 1.01E−05 | 486 | 1.57E−08 | 357 | 3.66E−07 |
| 84 | 1985 | 2.20E−08 | 1472 | 4.95E−09 | 1951 | 1.03E−05 | 1708 | 1.57E−08 | 36 | 3.75E−07 |
| 85 | 502 | 2.23E−08 | 1666 | 4.96E−09 | 2322 | 1.08E−05 | 2155 | 1.66E−08 | 494 | 3.78E−07 |
| 86 | 1973 | 2.29E−08 | 2155 | 5.06E−09 | 641 | 1.10E−05 | 192 | 1.68E−08 | 1658 | 3.84E−07 |
| 87 | 992 | 2.30E−08 | 2215 | 5.20E−09 | 1961 | 1.12E−05 | 1175 | 1.68E−08 | 818 | 3.86E−07 |
| 88 | 1009 | 2.30E−08 | 2322 | 5.22E−09 | 1357 | 1.13E−05 | 2320 | 1.69E−08 | 1469 | 3.86E−07 |
| 89 | 495 | 2.41E−08 | 1672 | 5.39E−09 | 1938 | 1.13E−05 | 1137 | 1.71E−08 | 681 | 3.89E−07 |
| 90 | 857 | 2.46E−08 | 567 | 5.52E−09 | 152 | 1.14E−05 | 1287 | 1.74E−08 | 2257 | 3.94E−07 |
| 91 | 2125 | 2.69E−08 | 199 | 5.55E−09 | 2321 | 1.14E−05 | 2091 | 1.81E−08 | 1831 | 4.08E−07 |
| 92 | 36 | 2.77E−08 | 544 | 5.56E−09 | 1872 | 1.15E−05 | 1086 | 1.84E−08 | 2156 | 4.27E−07 |
| 93 | 1566 | 2.79E−08 | 484 | 5.78E−09 | 1358 | 1.20E−05 | 873 | 1.89E−08 | 592 | 4.29E−07 |
| 94 | 545 | 2.92E−08 | 1917 | 5.87E−09 | 639 | 1.22E−05 | 1445 | 1.89E−08 | 284 | 4.32E−07 |
| 95 | 2308 | 2.98E−08 | 486 | 5.98E−09 | 77 | 1.23E−05 | 1594 | 1.89E−08 | 436 | 4.36E−07 |
| 96 | 332 | 3.13E−08 | 432 | 6.06E−09 | 647 | 1.23E−05 | 593 | 1.91E−08 | 446 | 4.37E−07 |
| 97 | 1722 | 3.18E−08 | 1810 | 6.07E−09 | 1628 | 1.25E−05 | 278 | 1.92E−08 | 1274 | 4.43E−07 |
| 98 | 1628 | 3.19E−08 | 759 | 6.19E−09 | 2195 | 1.27E−05 | 1015 | 2.00E−08 | 79 | 4.44E−07 |
| 99 | 1831 | 3.20E−08 | 1934 | 6.32E−09 | 274 | 1.34E−05 | 935 | 2.01E−08 | 617 | 4.51E−07 |
| [symbol] 00 | 2232 | 3.23E−08 | 573 | 6.60E−09 | 593 | 1.36E−05 | 1107 | 2.02E−08 | 393 | 4.70E−07 |

| | | | |
|---|---|---|---|
| 1RQ2 | 1UZR | 1ZAU | 2C27 |

| [symbol] ank | Drug ID | Calculated Ki | Drug ID | Calculated Ki | Drug ID | Calculated Ki | Drug ID | Calculated Ki |
|---|---|---|---|---|---|---|---|---|
| 1 | 2303 | 1.61E−09 | 551 | 4.54E−08 | 893 | 2.85E−11 | 274 | 3.43E−10 |
| 2 | 2321 | 1.75E−09 | 2232 | 2.12E−07 | 637 | 2.54E−10 | 1585 | 6.04E−10 |
| 3 | 2330 | 2.65E−09 | 550 | 2.38E−07 | 1921 | 2.92E−10 | 2216 | 9.56E−10 |
| 4 | 2287 | 3.39E−09 | 586 | 3.90E−07 | 482 | 3.52E−10 | 492 | 1.26E−09 |
| 5 | 2216 | 3.41E−09 | 553 | 4.09E−07 | 2174 | 4.81E−10 | 1256 | 1.33E−09 |
| 6 | 759 | 5.60E−09 | 546 | 4.65E−07 | 490 | 6.70E−10 | 1257 | 1.65E−09 |
| 7 | 274 | 6.09E−09 | 547 | 5.65E−07 | 818 | 6.77E−10 | 2174 | 2.06E−09 |
| 8 | 1921 | 8.87E−09 | 1899 | 6.14E−07 | 1805 | 6.97E−10 | 1710 | 2.18E−09 |
| 9 | 893 | 8.94E−09 | 2295 | 6.32E−07 | 685 | 8.19E−10 | 880 | 2.43E−09 |
| 10 | 1585 | 8.94E−09 | 1246 | 8.06E−07 | 2303 | 8.23E−10 | 436 | 2.50E−09 |
| 11 | 449 | 9.41E−09 | 593 | 8.27E−07 | 1951 | 9.63E−10 | 505 | 3.19E−09 |
| 12 | 232 | 9.66E−09 | 1866 | 8.29E−07 | 552 | 1.14E−09 | 2289 | 4.25E−09 |
| 13 | 2288 | 9.82E−09 | 758 | 8.61E−07 | 566 | 1.40E−09 | 2172 | 4.27E−09 |
| 14 | 702 | 1.16E−08 | 637 | 8.91E−07 | 2330 | 1.73E−09 | 448 | 4.46E−09 |
| 15 | 1934 | 1.27E−08 | 36 | 1.15E−06 | 2216 | 1.93E−09 | 1250 | 4.61E−09 |
| 16 | 552 | 1.29E−08 | 2089 | 1.17E−06 | 485 | 2.30E−09 | 2303 | 5.20E−09 |
| 17 | 492 | 1.46E−08 | 893 | 1.26E−06 | 488 | 2.98E−09 | 1594 | 5.70E−09 |
| 18 | 155 | 1.53E−08 | 642 | 1.27E−06 | 2089 | 3.68E−09 | 499 | 5.76E−09 |
| 19 | 1680 | 1.64E−08 | 2306 | 1.28E−06 | 484 | 3.69E−09 | 1089 | 6.06E−09 |
| 20 | 1 | 1.67E−08 | 1951 | 1.32E−06 | 36 | 3.76E−09 | 1834 | 6.53E−09 |
| 21 | 1445 | 1.73E−08 | 2303 | 1.36E−06 | 553 | 4.04E−09 | 346 | 7.20E−09 |
| 22 | 553 | 1.74E−08 | 1 | 1.41E−06 | 1198 | 4.09E−09 | 545 | 7.93E−09 |
| 23 | 1721 | 1.81E−08 | 517 | 1.41E−06 | 1469 | 4.47E−09 | 2321 | 8.42E−09 |
| 24 | 164 | 2.32E−08 | 448 | 1.45E−06 | 495 | 4.52E−09 | 488 | 8.53E−09 |
| 25 | 462 | 2.44E−08 | 2216 | 1.45E−06 | 2287 | 4.76E−09 | 478 | 8.91E−09 |
| 26 | 489 | 2.84E−08 | 643 | 1.46E−06 | 2289 | 5.08E−09 | 577 | 9.13E−09 |
| 27 | 2322 | 2.97E−08 | 434 | 1.51E−06 | 546 | 5.71E−09 | 1248 | 9.19E−09 |
| 28 | 436 | 3.02E−08 | 591 | 1.56E−06 | 759 | 5.75E−09 | 2023 | 9.55E−09 |
| 29 | 2009 | 3.03E−08 | 1860 | 1.57E−06 | 603 | 5.84E−09 | 1125 | 9.82E−09 |
| 30 | 637 | 3.26E−08 | 1968 | 1.61E−06 | 1462 | 5.92E−09 | 310 | 1.02E−08 |
| 31 | 548 | 3.47E−08 | 463 | 1.67E−06 | 2009 | 6.20E−09 | 2308 | 1.04E−08 |
| 32 | 445 | 3.64E−08 | 2289 | 1.67E−06 | 1 | 6.37E−09 | 2055 | 1.05E−08 |
| 33 | 486 | 3.77E−08 | 2287 | 1.73E−06 | 2134 | 6.69E−09 | 552 | 1.07E−08 |
| 34 | 472 | 4.21E−08 | 1017 | 1.91E−06 | 1497 | 6.84E−09 | 1896 | 1.07E−08 |
| 35 | 2289 | 4.26E−08 | 2302 | 1.92E−06 | 1570 | 7.36E−09 | 462 | 1.12E−08 |
| 36 | 551 | 4.35E−08 | 393 | 1.99E−06 | 499 | 7.41E−09 | 1420 | 1.15E−08 |
| 37 | 550 | 4.40E−08 | 1973 | 2.06E−06 | 479 | 8.33E−09 | 550 | 1.16E−08 |
| 38 | 71 | 4.54E−08 | 282 | 2.10E−06 | 2232 | 8.38E−09 | 491 | 1.19E−08 |
| 39 | 478 | 4.58E−08 | 603 | 2.13E−06 | 1249 | 8.47E−09 | 1450 | 1.39E−08 |
| 40 | 1679 | 5.46E−08 | 17 | 2.17E−06 | 550 | 9.07E−09 | 486 | 1.46E−08 |
| 41 | 1274 | 5.57E−08 | 544 | 2.17E−06 | 586 | 9.50E−09 | 1154 | 1.52E−08 |
| 42 | 880 | 5.71E−08 | 1332 | 2.22E−06 | 2125 | 1.03E−08 | 1274 | 1.52E−08 |
| 43 | 823 | 5.73E−08 | 163 | 2.24E−06 | 1559 | 1.05E−08 | 472 | 1.55E−08 |
| 44 | 448 | 6.01E−08 | 618 | 2.32E−06 | 492 | 1.08E−08 | 823 | 1.58E−08 |
| 45 | 473 | 6.31E−08 | 1921 | 2.32E−06 | 551 | 1.17E−08 | 889 | 1.63E−08 |
| 46 | 1260 | 6.37E−08 | 1117 | 2.35E−06 | 1834 | 1.26E−08 | 450 | 1.66E−08 |
| 47 | 944 | 6.57E−08 | 457 | 2.37E−06 | 545 | 1.36E−08 | 1 | 1.68E−08 |
| 48 | 546 | 6.97E−08 | 270 | 2.41E−06 | 486 | 1.37E−08 | 1086 | 1.76E−08 |
| 49 | 2301 | 7.13E−08 | 1033 | 2.45E−06 | 1548 | 1.38E−08 | 546 | 1.77E−08 |
| 50 | 1462 | 7.22E−08 | 928 | 2.53E−06 | 1585 | 1.43E−08 | 2232 | 1.77E−08 |
| 51 | 681 | 7.34E−08 | 1837 | 2.56E−06 | 567 | 1.45E−08 | 502 | 1.84E−08 |
| 52 | 1672 | 7.38E−08 | 979 | 2.62E−06 | 1973 | 1.46E−08 | 1908 | 1.85E−08 |
| 53 | 1512 | 7.46E−08 | 725 | 2.63E−06 | 314 | 1.52E−08 | 449 | 1.87E−08 |
| 54 | 1951 | 7.48E−08 | 507 | 2.68E−06 | 476 | 1.59E−08 | 1973 | 1.87E−08 |
| 55 | 1250 | 7.65E−08 | 556 | 2.69E−06 | 548 | 1.66E−08 | 2288 | 1.90E−08 |
| 56 | 1622 | 7.79E−08 | 577 | 2.72E−06 | 127 | 1.68E−08 | 1951 | 2.05E−08 |
| 57 | 867 | 7.80E−08 | 1815 | 2.83E−06 | 449 | 1.85E−08 | 845 | 2.12E−08 |
| 58 | 882 | 7.86E−08 | 596 | 2.96E−06 | 487 | 1.88E−08 | 1647 | 2.15E−08 |
| 59 | 1785 | 8.04E−08 | 628 | 2.98E−06 | 1767 | 1.90E−08 | 1989 | 2.16E−08 |
| 60 | 1944 | 8.04E−08 | 936 | 2.98E−06 | 483 | 1.95E−08 | 553 | 2.21E−08 |
| 61 | 438 | 8.06E−08 | 450 | 3.01E−06 | 975 | 2.00E−08 | 216 | 2.28E−08 |
| 62 | 993 | 8.17E−08 | 1133 | 3.02E−06 | 1117 | 2.02E−08 | 702 | 2.36E−08 |
| 63 | 115 | 8.36E−08 | 616 | 3.05E−06 | 521 | 2.03E−08 | 164 | 2.43E−08 |
| 64 | 593 | 8.72E−08 | 612 | 3.11E−06 | 274 | 2.15E−08 | 890 | 2.47E−08 |
| 65 | 467 | 8.87E−08 | 529 | 3.14E−06 | 2288 | 2.22E−08 | 496 | 2.52E−08 |
| 66 | 1985 | 8.93E−08 | 155 | 3.20E−06 | 1908 | 2.30E−08 | 2287 | 2.66E−08 |
| 67 | 554 | 9.50E−08 | 1431 | 3.24E−06 | 1710 | 2.38E−08 | 711 | 2.85E−08 |
| 68 | 877 | 9.56E−08 | 571 | 3.26E−06 | 593 | 2.40E−08 | 1748 | 2.88E−08 |
| 69 | 605 | 9.62E−08 | 1322 | 3.27E−06 | 1125 | 2.41E−08 | 818 | 3.07E−08 |
| 70 | 2343 | 9.63E−08 | 1492 | 3.38E−06 | 670 | 2.48E−08 | 1603 | 3.13E−08 |
| 71 | 1576 | 9.90E−08 | 1295 | 3.46E−06 | 2300 | 2.51E−08 | 538 | 3.19E−08 |
| 72 | 661 | 1.01E−07 | 1198 | 3.47E−06 | 334 | 2.69E−08 | 1076 | 3.35E−08 |
| 73 | 1947 | 1.01E−07 | 502 | 3.54E−06 | 478 | 2.75E−08 | 1947 | 3.37E−08 |
| 74 | 460 | 1.02E−07 | 386 | 3.66E−06 | 2018 | 2.75E−08 | 554 | 3.43E−08 |
| 75 | 2136 | 1.02E−07 | 3 | 3.69E−06 | 501 | 2.77E−08 | 548 | 3.45E−08 |
| 76 | 1634 | 1.06E−07 | 615 | 3.77E−06 | 350 | 2.84E−08 | 27 | 3.52E−08 |
| 77 | 1631 | 1.07E−07 | 2065 | 3.82E−06 | 822 | 2.88E−08 | 348 | 3.54E−08 |
| 78 | 350 | 1.09E−07 | 1919 | 3.83E−06 | 1205 | 2.89E−08 | 2322 | 3.59E−08 |
| 79 | 663 | 1.10E−07 | 312 | 3.86E−06 | 1253 | 2.89E−08 | 1418 | 3.61E−08 |
| 80 | 545 | 1.14E−07 | 617 | 3.86E−06 | 1777 | 2.90E−08 | 127 | 3.62E−08 |
| 81 | 27 | 1.17E−07 | 971 | 3.86E−06 | 2156 | 3.01E−08 | 1287 | 3.70E−08 |
| 82 | 505 | 1.18E−07 | 1844 | 3.94E−06 | 1274 | 3.04E−08 | 2336 | 3.73E−08 |
| 83 | 2125 | 1.23E−07 | 357 | 3.95E−06 | 2213 | 3.04E−08 | 1306 | 3.77E−08 |
| 84 | 1154 | 1.24E−07 | 871 | 3.95E−06 | 489 | 3.08E−08 | 567 | 3.78E−08 |
| 85 | 1973 | 1.25E−07 | 756 | 3.97E−06 | 1569 | 3.13E−08 | 236 | 3.83E−08 |
| 86 | 2171 | 1.28E−07 | 427 | 3.98E−06 | 1722 | 3.16E−08 | 1355 | 3.93E−08 |
| 87 | 573 | 1.29E−07 | 875 | 4.00E−06 | 480 | 3.36E−08 | 1708 | 4.00E−08 |
| 88 | 760 | 1.30E−07 | 657 | 4.01E−06 | 1108 | 3.52E−08 | 935 | 4.06E−08 |
| 89 | 1748 | 1.30E−07 | 1683 | 4.05E−06 | 882 | 3.53E−08 | 1652 | 4.06E−08 |
| 90 | 346 | 1.34E−07 | 1806 | 4.14E−06 | 2172 | 3.54E−08 | 131 | 4.08E−08 |
| 91 | 1301 | 1.34E−07 | 534 | 4.15E−06 | 2309 | 3.55E−08 | 551 | 4.17E−08 |
| 92 | 483 | 1.35E−07 | 2174 | 4.16E−06 | 1596 | 3.61E−08 | 1629 | 4.19E−08 |
| 93 | 1248 | 1.36E−07 | 1570 | 4.17E−06 | 477 | 3.67E−08 | 1607 | 4.20E−08 |
| 94 | 2089 | 1.37E−07 | 1917 | 4.21E−06 | 1016 | 3.94E−08 | 1666 | 4.20E−08 |
| 95 | 577 | 1.40E−07 | 1915 | 4.22E−06 | 463 | 3.97E−08 | 566 | 4.28E−08 |
| 96 | 1176 | 1.40E−07 | 1985 | 4.22E−06 | 1985 | 4.16E−08 | 601 | 4.37E−08 |
| 97 | 2262 | 1.42E−07 | 545 | 4.24E−06 | 1666 | 4.41E−08 | 893 | 4.43E−08 |
| 98 | 1548 | 1.43E−07 | 1912 | 4.30E−06 | 1680 | 4.53E−08 | 477 | 4.44E−08 |
| 99 | 366 | 1.46E−07 | 458 | 4.34E−06 | 2322 | 4.66E−08 | 71 | 4.46E−08 |
| [symbol] 00 | 1910 | 1.46E−07 | 1534 | 4.34E−06 | 27 | 4.68E−08 | 637 | 4.72E−08 |

[symbol]

EED 2

| | 1F61 | | 1GR0 | | 1IDS | | 1N2E | | 1N8W | |
|---|---|---|---|---|---|---|---|---|---|---|
| [symbol] ank | Drug ID | Calculated Ki | Drug ID | Calculated Ki | Drug ID | Calculated Ki | Drug ID | Calculated Ki | Drug ID | Calculated Ki |
| 1 | 1462 | 6.79E−13 | 2303 | 9.33E−15 | 1647 | 1.22E−10 | 1253 | 4.34E−13 | 2303 | 5.25E−10 |
| 2 | 2303 | 1.15E−12 | 893 | 7.10E−12 | 1584 | 1.50E−07 | 560 | 2.56E−11 | 1921 | 1.21E−08 |
| 3 | 685 | 1.27E−12 | 2287 | 1.77E−11 | 1909 | 1.57E−07 | 275 | 1.49E−10 | 1117 | 1.38E−08 |
| 4 | 1921 | 1.80E−12 | 1559 | 2.53E−11 | 1979 | 3.42E−07 | 274 | 1.65E−10 | 1951 | 1.75E−08 |
| 5 | 670 | 7.28E−12 | 492 | 4.16E−11 | 1921 | 4.30E−07 | 1921 | 2.55E−10 | 637 | 1.87E−08 |
| 6 | 1065 | 8.44E−12 | 1921 | 4.32E−11 | 550 | 4.87E−07 | 1780 | 5.80E−10 | 1973 | 2.36E−08 |
| 7 | 1570 | 1.17E−11 | 1198 | 4.43E−11 | 1780 | 9.55E−07 | 472 | 7.57E−10 | 155 | 2.63E−08 |
| 8 | 1469 | 1.32E−11 | 548 | 5.84E−11 | 2216 | 1.08E−06 | 499 | 8.26E−10 | 2125 | 2.81E−08 |
| 9 | 1565 | 4.23E−11 | 670 | 7.19E−11 | 2174 | 1.16E−06 | 318 | 8.68E−10 | 2216 | 3.01E−08 |
| 10 | 2262 | 5.11E−11 | 1565 | 8.76E−11 | 36 | 1.62E−06 | 2321 | 9.63E−10 | 2018 | 3.10E−08 |
| 11 | 1607 | 5.99E−11 | 919 | 1.24E−10 | 637 | 1.74E−06 | 2330 | 1.03E−09 | 550 | 3.14E−08 |
| 12 | 919 | 9.02E−11 | 1117 | 1.24E−10 | 1 | 2.03E−06 | 551 | 1.10E−09 | 893 | 3.24E−08 |
| 13 | 546 | 9.26E−11 | 552 | 1.78E−10 | 502 | 2.38E−06 | 1257 | 1.22E−09 | 551 | 3.30E−08 |
| 14 | 2287 | 1.41E−10 | 245 | 1.84E−10 | 1767 | 2.45E−06 | 2308 | 1.54E−09 | 2321 | 3.74E−08 |
| 15 | 1364 | 2.13E−10 | 553 | 1.91E−10 | 71 | 2.53E−06 | 2216 | 1.55E−09 | 274 | 3.82E−08 |
| 16 | 2174 | 2.57E−10 | 1951 | 2.00E−10 | 1309 | 2.53E−06 | 1920 | 1.56E−09 | 1607 | 4.01E−08 |
| 17 | 553 | 2.69E−10 | 2328 | 3.46E−10 | 2289 | 2.70E−06 | 1777 | 1.81E−09 | 548 | 4.20E−08 |
| 18 | 1117 | 2.94E−10 | 1915 | 3.51E−10 | 545 | 2.90E−06 | 545 | 1.99E−09 | 2287 | 4.33E−08 |
| 19 | 637 | 3.03E−10 | 482 | 3.52E−10 | 1839 | 2.98E−06 | 1666 | 2.15E−09 | 1680 | 5.91E−08 |
| 20 | 2053 | 4.75E−10 | 2174 | 3.55E−10 | 1968 | 3.01E−06 | 1951 | 2.93E−09 | 2174 | 6.20E−08 |
| 21 | 877 | 5.02E−10 | 2300 | 3.66E−10 | 1721 | 3.19E−06 | 2329 | 3.20E−09 | 553 | 6.25E−08 |
| 22 | 893 | 5.77E−10 | 2321 | 4.09E−10 | 506 | 3.21E−06 | 205 | 3.21E−09 | 2058 | 6.35E−08 |
| 23 | 490 | 6.19E−10 | 685 | 4.19E−10 | 553 | 3.50E−06 | 479 | 3.53E−09 | 2322 | 6.54E−08 |
| 24 | 2330 | 6.26E−10 | 488 | 4.52E−10 | 551 | 3.51E−06 | 1585 | 3.64E−09 | 492 | 6.71E−08 |
| 25 | 552 | 7.32E−10 | 491 | 5.12E−10 | 9 | 3.57E−06 | 492 | 3.73E−09 | 350 | 6.76E−08 |
| 26 | 2259 | 1.06E−09 | 554 | 5.19E−10 | 533 | 3.59E−06 | 236 | 3.76E−09 | 1462 | 6.86E−08 |
| 27 | 491 | 1.29E−09 | 1138 | 5.19E−10 | 822 | 3.91E−06 | 2289 | 3.82E−09 | 17 | 7.69E−08 |
| 28 | 2216 | 1.37E−09 | 1680 | 5.35E−10 | 278 | 3.98E−06 | 1569 | 4.11E−09 | 603 | 7.73E−08 |
| 29 | 2171 | 1.51E−09 | 2330 | 5.80E−10 | 482 | 4.31E−06 | 553 | 4.17E−09 | 1777 | 7.85E−08 |
| 30 | 1680 | 1.54E−09 | 2074 | 5.82E−10 | 2320 | 4.35E−06 | 2288 | 4.25E−09 | 2134 | 8.05E−08 |
| 31 | 1438 | 1.93E−09 | 1065 | 6.05E−10 | 993 | 4.37E−06 | 679 | 4.32E−09 | 1985 | 8.28E−08 |
| 32 | 399 | 1.98E−09 | 2216 | 6.17E−10 | 1437 | 4.37E−06 | 548 | 4.49E−09 | 2289 | 8.53E−08 |
| 33 | 1915 | 2.10E−09 | 2123 | 6.52E−10 | 334 | 4.40E−06 | 2301 | 4.53E−09 | 2301 | 8.97E−08 |
| 34 | 1198 | 2.20E−09 | 318 | 6.57E−10 | 1716 | 4.47E−06 | 1908 | 4.69E−09 | 1989 | 9.30E−08 |
| 35 | 379 | 2.22E−09 | 1732 | 7.17E−10 | 618 | 4.52E−06 | 663 | 4.82E−09 | 245 | 9.58E−08 |
| 36 | 2009 | 2.35E−09 | 2343 | 8.31E−10 | 2155 | 4.68E−06 | 488 | 5.04E−09 | 439 | 1.02E−07 |
| 37 | 2295 | 2.35E−09 | 490 | 9.06E−10 | 1939 | 5.20E−06 | 993 | 5.28E−09 | 521 | 1.03E−07 |
| 38 | 232 | 2.53E−09 | 1989 | 9.32E−10 | 1783 | 5.24E−06 | 2171 | 5.37E−09 | 164 | 1.26E−07 |
| 39 | 496 | 2.70E−09 | 603 | 9.49E−10 | 566 | 5.30E−06 | 893 | 5.44E−09 | 501 | 1.34E−07 |
| 40 | 482 | 2.75E−09 | 478 | 9.62E−10 | 348 | 5.32E−06 | 1973 | 5.50E−09 | 271 | 1.35E−07 |
| 41 | 2018 | 3.04E−09 | 1438 | 1.01E−09 | 1634 | 5.58E−06 | 1176 | 5.61E−09 | 1332 | 1.44E−07 |
| 42 | 1860 | 3.13E−09 | 1223 | 1.02E−09 | 1663 | 5.59E−06 | 208 | 5.94E−09 | 2300 | 1.44E−07 |
| 43 | 548 | 3.26E−09 | 550 | 1.14E−09 | 552 | 5.90E−06 | 566 | 6.08E−09 | 1357 | 1.62E−07 |
| 44 | 1556 | 3.49E−09 | 505 | 1.17E−09 | 1140 | 5.96E−06 | 711 | 6.35E−09 | 567 | 1.63E−07 |
| 45 | 1323 | 3.58E−09 | 1973 | 1.20E−09 | 590 | 5.97E−06 | 867 | 6.40E−09 | 759 | 1.68E−07 |
| 46 | 1561 | 4.00E−09 | 1469 | 1.25E−09 | 2055 | 6.09E−06 | 714 | 6.48E−09 | 554 | 1.71E−07 |
| 47 | 2177 | 4.92E−09 | 485 | 1.30E−09 | 818 | 6.20E−06 | 1497 | 6.95E−09 | 482 | 1.75E−07 |
| 48 | 1631 | 4.95E−09 | 2156 | 1.33E−09 | 736 | 6.28E−06 | 1603 | 7.08E−09 | 552 | 1.78E−07 |
| 49 | 488 | 5.23E−09 | 546 | 1.34E−09 | 1951 | 6.36E−06 | 637 | 7.69E−09 | 670 | 1.79E−07 |
| 50 | 904 | 5.42E−09 | 127 | 1.38E−09 | 755 | 6.38E−06 | 1418 | 7.98E−09 | 574 | 1.92E−07 |
| 51 | 1246 | 5.81E−09 | 493 | 1.44E−09 | 563 | 6.70E−06 | 2174 | 8.30E−09 | 1666 | 1.92E−07 |
| 52 | 1708 | 6.09E−09 | 545 | 1.44E−09 | 567 | 7.09E−06 | 1834 | 8.42E−09 | 1837 | 1.92E−07 |
| 53 | 504 | 6.74E−09 | 489 | 1.47E−09 | 1960 | 7.12E−06 | 2303 | 8.45E−09 | 613 | 1.99E−07 |
| 54 | 1658 | 7.00E−09 | 1287 | 1.49E−09 | 1973 | 7.12E−06 | 552 | 8.80E−09 | 1672 | 2.09E−07 |
| 55 | 238 | 7.67E−09 | 271 | 1.57E−09 | 621 | 7.17E−06 | 2009 | 8.81E−09 | 593 | 2.10E−07 |
| 56 | 702 | 8.03E−09 | 1521 | 1.62E−09 | 2078 | 7.37E−06 | 448 | 8.84E−09 | 545 | 2.12E−07 |
| 57 | 2074 | 8.18E−09 | 494 | 1.66E−09 | 1985 | 7.54E−06 | 310 | 9.17E−09 | 491 | 2.14E−07 |
| 58 | 1552 | 8.25E−09 | 1462 | 1.71E−09 | 1525 | 7.57E−06 | 494 | 9.29E−09 | 546 | 2.15E−07 |
| 59 | 618 | 8.57E−09 | 2329 | 1.78E−09 | 1807 | 7.74E−06 | 486 | 9.43E−09 | 2154 | 2.25E−07 |
| 60 | 1934 | 9.12E−09 | 637 | 1.79E−09 | 628 | 7.85E−06 | 1748 | 9.63E−09 | 2288 | 2.31E−07 |
| 61 | 551 | 9.42E−09 | 1570 | 1.90E−09 | 549 | 7.92E−06 | 554 | 9.76E−09 | 736 | 2.35E−07 |
| 62 | 2046 | 9.83E−09 | 2239 | 2.21E−09 | 1067 | 8.15E−06 | 759 | 1.00E−08 | 1 | 2.37E−07 |
| 63 | 2321 | 9.84E−09 | 2289 | 2.22E−09 | 450 | 8.26E−06 | 1762 | 1.02E−08 | 1938 | 2.52E−07 |
| 64 | 1609 | 1.02E−08 | 481 | 2.40E−09 | 1037 | 8.58E−06 | 1594 | 1.04E−08 | 586 | 2.56E−07 |
| 65 | 1951 | 1.02E−08 | 591 | 2.41E−09 | 472 | 8.71E−06 | 1680 | 1.05E−08 | 566 | 2.60E−07 |
| 66 | 1641 | 1.06E−08 | 232 | 2.67E−09 | 1823 | 8.86E−06 | 573 | 1.06E−08 | 445 | 2.62E−07 |
| 67 | 2289 | 1.09E−08 | 1125 | 2.85E−09 | 488 | 8.91E−06 | 2125 | 1.06E−08 | 379 | 2.64E−07 |
| 68 | 1155 | 1.15E−08 | 2009 | 3.04E−09 | 1748 | 8.98E−06 | 702 | 1.09E−08 | 822 | 2.71E−07 |
| 69 | 246 | 1.24E−08 | 2089 | 3.08E−09 | 17 | 9.05E−06 | 1390 | 1.10E−08 | 867 | 2.84E−07 |
| 70 | 1866 | 1.27E−08 | 2288 | 3.27E−09 | 547 | 9.10E−06 | 271 | 1.13E−08 | 1774 | 2.85E−07 |
| 71 | 27 | 1.37E−08 | 1968 | 3.30E−09 | 2309 | 9.15E−06 | 1099 | 1.13E−08 | 1418 | 2.86E−07 |
| 72 | 2288 | 1.37E−08 | 480 | 3.42E−09 | 984 | 9.27E−06 | 550 | 1.20E−08 | 36 | 3.03E−07 |
| 73 | 1 | 1.43E−08 | 2232 | 3.42E−09 | 681 | 9.30E−06 | 1417 | 1.25E−08 | 115 | 3.04E−07 |
| 74 | 492 | 1.43E−08 | 2134 | 3.57E−09 | 1453 | 9.44E−06 | 1647 | 1.25E−08 | 1967 | 3.06E−07 |
| 75 | 499 | 1.46E−08 | 238 | 3.97E−09 | 310 | 9.46E−06 | 199 | 1.27E−08 | 1476 | 3.15E−07 |
| 76 | 431 | 1.51E−08 | 1666 | 4.24E−09 | 605 | 9.54E−06 | 436 | 1.27E−08 | 939 | 3.21E−07 |
| 77 | 1972 | 1.52E−08 | 486 | 4.33E−09 | 1089 | 9.87E−06 | 480 | 1.28E−08 | 1437 | 3.31E−07 |
| 78 | 495 | 1.58E−08 | 449 | 4.34E−09 | 1611 | 9.96E−06 | 2215 | 1.29E−08 | 544 | 3.41E−07 |
| 79 | 332 | 1.63E−08 | 2018 | 4.38E−09 | 1969 | 1.04E−05 | 1250 | 1.31E−08 | 357 | 3.49E−07 |
| 80 | 386 | 1.74E−08 | 2332 | 4.43E−09 | 546 | 1.05E−05 | 1395 | 1.32E−08 | 882 | 3.57E−07 |
| 81 | 873 | 1.74E−08 | 502 | 4.59E−09 | 1810 | 1.08E−05 | 1831 | 1.33E−08 | 429 | 3.60E−07 |
| 82 | 205 | 1.77E−08 | 604 | 4.59E−09 | 548 | 1.09E−05 | 2239 | 1.35E−08 | 681 | 3.64E−07 |
| 83 | 155 | 1.79E−08 | 2322 | 4.63E−09 | 641 | 1.10E−05 | 17 | 1.41E−08 | 1469 | 3.74E−07 |
| 84 | 939 | 1.80E−08 | 1255 | 4.67E−09 | 1357 | 1.11E−05 | 490 | 1.45E−08 | 449 | 3.90E−07 |
| 85 | 617 | 1.82E−08 | 439 | 4.69E−09 | 1961 | 1.12E−05 | 2277 | 1.45E−08 | 1658 | 3.92E−07 |
| 86 | 502 | 2.01E−08 | 1472 | 4.74E−09 | 1938 | 1.13E−05 | 853 | 1.49E−08 | 2257 | 4.00E−07 |
| 87 | 350 | 2.10E−08 | 350 | 4.75E−09 | 152 | 1.16E−05 | 1205 | 1.52E−08 | 818 | 4.08E−07 |
| 88 | 1620 | 2.18E−08 | 346 | 4.77E−09 | 2284 | 1.17E−05 | 1641 | 1.53E−08 | 1086 | 4.11E−07 |
| 89 | 1985 | 2.21E−08 | 1672 | 5.13E−09 | 1872 | 1.18E−05 | 2154 | 1.53E−08 | 436 | 4.13E−07 |
| 90 | 503 | 2.29E−08 | 567 | 5.40E−09 | 1358 | 1.20E−05 | 603 | 1.54E−08 | 499 | 4.18E−07 |
| 91 | 346 | 2.30E−08 | 199 | 5.52E−09 | 647 | 1.27E−05 | 944 | 1.54E−08 | 284 | 4.31E−07 |
| 92 | 1009 | 2.31E−08 | 2215 | 5.55E−09 | 725 | 1.27E−05 | 1583 | 1.54E−08 | 617 | 4.48E−07 |
| 93 | 1973 | 2.31E−08 | 155 | 5.56E−09 | 2039 | 1.27E−05 | 247 | 1.55E−08 | 592 | 4.53E−07 |
| 94 | 449 | 2.40E−08 | 544 | 5.66E−09 | 1994 | 1.29E−05 | 1137 | 1.55E−08 | 393 | 4.70E−07 |
| 95 | 857 | 2.46E−08 | 759 | 5.69E−09 | 1628 | 1.32E−05 | 1634 | 1.57E−08 | 1223 | 4.75E−07 |
| 96 | 2125 | 2.49E−08 | 1641 | 5.74E−09 | 535 | 1.34E−05 | 1512 | 1.63E−08 | 1939 | 4.76E−07 |
| 97 | 36 | 2.66E−08 | 1679 | 5.83E−09 | 593 | 1.37E−05 | 1108 | 1.65E−08 | 536 | 4.88E−07 |
| 98 | 2156 | 2.82E−08 | 1831 | 5.84E−09 | 906 | 1.37E−05 | 192 | 1.69E−08 | 1274 | 4.96E−07 |
| 99 | 481 | 2.87E−08 | 1917 | 5.87E−09 | 1631 | 1.37E−05 | 1175 | 1.70E−08 | 2262 | 5.04E−07 |
| [symbol] 00 | 545 | 3.10E−08 | 2125 | 5.89E−09 | 374 | 1.39E−05 | 1086 | 1.72E−08 | 1831 | 5.08E−07 |

| | | | |
|---|---|---|---|
| 1RQ2 | 1UZR | 1ZAU | 2C27 |

| [symbol] ank | Drug ID | Calculated Ki | Drug ID | Calculated Ki | Drug ID | Calculated Ki | Drug ID | Calculated Ki |
|---|---|---|---|---|---|---|---|---|
| 1 | 2303 | 1.60E−09 | 551 | 6.33E−08 | 637 | 2.35E−10 | 1585 | 2.65E−10 |
| 2 | 2321 | 1.70E−09 | 2232 | 1.96E−07 | 482 | 3.23E−10 | 274 | 3.83E−10 |
| 3 | 2287 | 2.01E−09 | 586 | 3.90E−07 | 490 | 6.41E−10 | 2216 | 9.27E−10 |
| 4 | 2216 | 3.74E−09 | 553 | 4.16E−07 | 818 | 6.47E−10 | 1257 | 1.29E−09 |
| 5 | 759 | 5.90E−09 | 550 | 4.50E−07 | 1469 | 7.48E−10 | 492 | 1.31E−09 |
| 6 | 1921 | 7.11E−09 | 1899 | 4.71E−07 | 2303 | 8.18E−10 | 1256 | 1.35E−09 |
| 7 | 232 | 7.99E−09 | 546 | 4.88E−07 | 919 | 9.47E−10 | 505 | 1.71E−09 |
| 8 | 155 | 9.65E−09 | 547 | 5.67E−07 | 2174 | 9.86E−10 | 1834 | 2.75E−09 |
| 9 | 2288 | 9.82E−09 | 521 | 6.52E−07 | 552 | 1.25E−09 | 845 | 2.99E−09 |
| 10 | 449 | 1.09E−08 | 637 | 7.64E−07 | 893 | 1.29E−09 | 1710 | 3.08E−09 |
| 11 | 552 | 1.29E−08 | 1246 | 8.16E−07 | 566 | 1.52E−09 | 236 | 4.25E−09 |
| 12 | 1585 | 1.29E−08 | 593 | 8.29E−07 | 2089 | 1.75E−09 | 499 | 4.30E−09 |
| 13 | 1 | 1.37E−08 | 661 | 8.64E−07 | 2216 | 1.92E−09 | 448 | 4.43E−09 |
| 14 | 893 | 1.51E−08 | 758 | 8.94E−07 | 1117 | 2.50E−09 | 1089 | 4.73E−09 |
| 15 | 1951 | 1.55E−08 | 1866 | 9.30E−07 | 2134 | 2.93E−09 | 488 | 5.07E−09 |
| 16 | 553 | 1.86E−08 | 1973 | 1.06E−06 | 488 | 3.24E−09 | 2303 | 5.20E−09 |
| 17 | 1721 | 1.90E−08 | 1860 | 1.08E−06 | 1565 | 3.71E−09 | 1250 | 5.28E−09 |
| 18 | 702 | 1.92E−08 | 552 | 1.11E−06 | 553 | 3.79E−09 | 2289 | 6.03E−09 |
| 19 | 1250 | 2.29E−08 | 2089 | 1.11E−06 | 36 | 3.83E−09 | 346 | 7.09E−09 |
| 20 | 2009 | 2.32E−08 | 2306 | 1.14E−06 | 759 | 3.86E−09 | 436 | 7.17E−09 |
| 21 | 274 | 2.46E−08 | 434 | 1.21E−06 | 1125 | 4.27E−09 | 545 | 7.55E−09 |
| 22 | 2322 | 2.60E−08 | 642 | 1.26E−06 | 495 | 4.55E−09 | 880 | 8.31E−09 |
| 23 | 681 | 2.62E−08 | 2289 | 1.28E−06 | 2289 | 5.08E−09 | 2174 | 8.51E−09 |
| 24 | 462 | 2.63E−08 | 2138 | 1.33E−06 | 1249 | 5.29E−09 | 577 | 8.75E−09 |
| 25 | 2162 | 2.91E−08 | 2303 | 1.37E−06 | 2287 | 6.16E−09 | 2330 | 8.81E−09 |
| 26 | 2174 | 3.01E−08 | 979 | 1.38E−06 | 685 | 6.23E−09 | 550 | 9.10E−09 |
| 27 | 993 | 3.09E−08 | 517 | 1.43E−06 | 603 | 6.55E−09 | 1896 | 9.78E−09 |
| 28 | 637 | 3.10E−08 | 643 | 1.44E−06 | 485 | 6.56E−09 | 815 | 9.79E−09 |
| 29 | 550 | 3.18E−08 | 2216 | 1.46E−06 | 1570 | 7.29E−09 | 310 | 9.89E−09 |
| 30 | 1512 | 3.43E−08 | 603 | 1.53E−06 | 479 | 7.59E−09 | 2023 | 9.97E−09 |
| 31 | 478 | 3.67E−08 | 591 | 1.55E−06 | 546 | 8.20E−09 | 486 | 1.01E−08 |
| 32 | 436 | 3.69E−08 | 1968 | 1.60E−06 | 2232 | 8.46E−09 | 2172 | 1.02E−08 |
| 33 | 445 | 3.72E−08 | 1951 | 1.66E−06 | 484 | 8.60E−09 | 2287 | 1.02E−08 |
| 34 | 1445 | 3.98E−08 | 725 | 1.76E−06 | 274 | 8.72E−09 | 2321 | 1.05E−08 |
| 35 | 1934 | 4.16E−08 | 1016 | 1.83E−06 | 1462 | 9.19E−09 | 552 | 1.07E−08 |
| 36 | 551 | 4.31E−08 | 544 | 1.89E−06 | 586 | 9.47E−09 | 2055 | 1.08E−08 |
| 37 | 2289 | 4.43E−08 | 457 | 1.94E−06 | 2156 | 9.87E−09 | 462 | 1.11E−08 |
| 38 | 71 | 4.75E−08 | 393 | 1.99E−06 | 1 | 1.02E−08 | 1594 | 1.12E−08 |
| 39 | 548 | 4.81E−08 | 282 | 2.10E−06 | 2125 | 1.04E−08 | 1420 | 1.19E−08 |
| 40 | 2262 | 5.38E−08 | 548 | 2.18E−06 | 548 | 1.17E−08 | 1973 | 1.27E−08 |
| 41 | 448 | 5.39E−08 | 17 | 2.20E−06 | 478 | 1.36E−08 | 484 | 1.33E−08 |
| 42 | 823 | 5.43E−08 | 163 | 2.23E−06 | 314 | 1.37E−08 | 2288 | 1.34E−08 |
| 43 | 1274 | 5.53E−08 | 618 | 2.27E−06 | 481 | 1.39E−08 | 1125 | 1.36E−08 |
| 44 | 944 | 5.96E−08 | 270 | 2.40E−06 | 567 | 1.45E−08 | 1450 | 1.36E−08 |
| 45 | 1666 | 6.01E−08 | 1033 | 2.46E−06 | 1548 | 1.48E−08 | 1989 | 1.36E−08 |
| 46 | 1576 | 6.15E−08 | 155 | 2.48E−06 | 2009 | 1.51E−08 | 823 | 1.38E−08 |
| 47 | 1679 | 6.22E−08 | 1837 | 2.56E−06 | 127 | 1.69E−08 | 1076 | 1.41E−08 |
| 48 | 1947 | 6.23E−08 | 1017 | 2.60E−06 | 1253 | 1.70E−08 | 1274 | 1.49E−08 |
| 49 | 1287 | 6.35E−08 | 1291 | 2.61E−06 | 499 | 1.72E−08 | 1154 | 1.53E−08 |
| 50 | 1785 | 6.68E−08 | 1566 | 2.62E−06 | 476 | 1.75E−08 | 449 | 1.56E−08 |
| 51 | 164 | 6.84E−08 | 556 | 2.69E−06 | 521 | 2.03E−08 | 1777 | 1.60E−08 |
| 52 | 486 | 7.06E−08 | 502 | 2.73E−06 | 1596 | 2.10E−08 | 889 | 1.61E−08 |
| 53 | 546 | 7.07E−08 | 1985 | 2.73E−06 | 593 | 2.26E−08 | 450 | 1.68E−08 |
| 54 | 1570 | 7.25E−08 | 1963 | 2.74E−06 | 1569 | 2.26E−08 | 1086 | 1.80E−08 |
| 55 | 1672 | 7.42E−08 | 1631 | 2.80E−06 | 477 | 2.27E−08 | 2232 | 1.83E−08 |
| 56 | 447 | 8.00E−08 | 928 | 2.82E−06 | 1205 | 2.34E−08 | 478 | 1.84E−08 |
| 57 | 2330 | 8.09E−08 | 500 | 2.85E−06 | 2300 | 2.57E−08 | 502 | 1.89E−08 |
| 58 | 2274 | 8.10E−08 | 936 | 2.88E−06 | 486 | 2.59E−08 | 546 | 1.93E−08 |
| 59 | 115 | 8.28E−08 | 1076 | 2.88E−06 | 2018 | 2.75E−08 | 472 | 1.96E−08 |
| 60 | 1607 | 8.31E−08 | 628 | 2.95E−06 | 480 | 2.79E−08 | 702 | 1.96E−08 |
| 61 | 1944 | 8.53E−08 | 1815 | 2.95E−06 | 350 | 2.83E−08 | 27 | 2.02E−08 |
| 62 | 489 | 8.60E−08 | 2287 | 2.97E−06 | 545 | 2.87E−08 | 1622 | 2.02E−08 |
| 63 | 882 | 8.63E−08 | 616 | 3.01E−06 | 1631 | 2.88E−08 | 553 | 2.12E−08 |
| 64 | 1634 | 8.64E−08 | 507 | 3.05E−06 | 492 | 2.90E−08 | 2308 | 2.18E−08 |
| 65 | 2301 | 8.66E−08 | 465 | 3.06E−06 | 550 | 2.91E−08 | 538 | 2.21E−08 |
| 66 | 1985 | 8.85E−08 | 529 | 3.12E−06 | 2213 | 2.91E−08 | 1908 | 2.25E−08 |
| 67 | 501 | 8.99E−08 | 596 | 3.13E−06 | 2288 | 2.91E−08 | 1603 | 2.31E−08 |
| 68 | 460 | 9.37E−08 | 969 | 3.23E−06 | 822 | 2.99E−08 | 1248 | 2.35E−08 |
| 69 | 593 | 9.49E−08 | 1 | 3.26E−06 | 1198 | 2.99E−08 | 1708 | 2.44E−08 |
| 70 | 554 | 9.62E−08 | 571 | 3.26E−06 | 2309 | 2.99E−08 | 890 | 2.46E−08 |
| 71 | 467 | 9.64E−08 | 1431 | 3.28E−06 | 1274 | 3.04E−08 | 1 | 2.76E−08 |
| 72 | 1631 | 9.75E−08 | 1322 | 3.30E−06 | 2155 | 3.06E−08 | 1748 | 2.85E−08 |
| 73 | 919 | 9.81E−08 | 1683 | 3.30E−06 | 551 | 3.09E−08 | 1951 | 2.94E−08 |
| 74 | 1248 | 1.01E−07 | 2302 | 3.30E−06 | 501 | 3.11E−08 | 1306 | 2.96E−08 |
| 75 | 545 | 1.02E−07 | 1492 | 3.31E−06 | 1438 | 3.11E−08 | 711 | 2.98E−08 |
| 76 | 350 | 1.03E−07 | 1295 | 3.46E−06 | 2330 | 3.22E−08 | 893 | 3.05E−08 |
| 77 | 661 | 1.04E−07 | 450 | 3.49E−06 | 1780 | 3.25E−08 | 818 | 3.15E−08 |
| 78 | 1117 | 1.06E−07 | 1009 | 3.54E−06 | 1722 | 3.27E−08 | 164 | 3.19E−08 |
| 79 | 477 | 1.07E−07 | 906 | 3.57E−06 | 483 | 3.30E−08 | 759 | 3.24E−08 |
| 80 | 605 | 1.13E−07 | 3 | 3.76E−06 | 487 | 3.31E−08 | 1418 | 3.25E−08 |
| 81 | 27 | 1.17E−07 | 386 | 3.80E−06 | 1108 | 3.31E−08 | 1629 | 3.36E−08 |
| 82 | 663 | 1.17E−07 | 615 | 3.80E−06 | 1585 | 3.32E−08 | 1652 | 3.38E−08 |
| 83 | 2320 | 1.17E−07 | 2065 | 3.82E−06 | 1666 | 3.38E−08 | 554 | 3.51E−08 |
| 84 | 2136 | 1.18E−07 | 1919 | 3.83E−06 | 813 | 3.44E−08 | 127 | 3.56E−08 |
| 85 | 1301 | 1.22E−07 | 357 | 3.84E−06 | 1497 | 3.45E−08 | 348 | 3.56E−08 |
| 86 | 483 | 1.23E−07 | 312 | 3.89E−06 | 882 | 3.49E−08 | 2322 | 3.57E−08 |
| 87 | 1748 | 1.23E−07 | 617 | 3.90E−06 | 2034 | 3.53E−08 | 1810 | 3.63E−08 |
| 88 | 573 | 1.28E−07 | 971 | 3.93E−06 | 1089 | 3.56E−08 | 1641 | 3.65E−08 |
| 89 | 577 | 1.29E−07 | 871 | 3.95E−06 | 334 | 3.63E−08 | 71 | 3.66E−08 |
| 90 | 760 | 1.29E−07 | 657 | 3.96E−06 | 670 | 3.65E−08 | 491 | 3.66E−08 |
| 91 | 1462 | 1.31E−07 | 2134 | 3.99E−06 | 1985 | 3.70E−08 | 637 | 3.75E−08 |
| 92 | 1973 | 1.33E−07 | 875 | 4.00E−06 | 1248 | 3.83E−08 | 2134 | 3.80E−08 |
| 93 | 494 | 1.34E−07 | 1616 | 4.04E−06 | 1287 | 3.94E−08 | 438 | 3.87E−08 |
| 94 | 1845 | 1.35E−07 | 795 | 4.13E−06 | 163 | 4.00E−08 | 1780 | 3.87E−08 |
| 95 | 276 | 1.37E−07 | 1806 | 4.13E−06 | 538 | 4.06E−08 | 567 | 3.93E−08 |
| 96 | 2125 | 1.38E−07 | 896 | 4.15E−06 | 2320 | 4.11E−08 | 1287 | 3.94E−08 |
| 97 | 346 | 1.39E−07 | 534 | 4.17E−06 | 463 | 4.24E−08 | 1654 | 3.98E−08 |
| 98 | 1989 | 1.44E−07 | 1921 | 4.20E−06 | 1620 | 4.65E−08 | 1607 | 3.99E−08 |
| 99 | 1910 | 1.45E−07 | 1917 | 4.21E−06 | 449 | 4.71E−08 | 1647 | 4.07E−08 |
| 100 | 1075 | 1.49E−07 | 1534 | 4.33E−06 | 1910 | 4.78E−08 | 131 | 4.08E−08 |

SEED 3

| | 1F61 | | 1GR0 | | 1IDS | | 1N2E | | 1N8W | |
|---|---|---|---|---|---|---|---|---|---|---|
| [symbol] ank | Drug ID | Calculated Ki | Drug ID | Calculated Ki | Drug ID | Calculated Ki | Drug ID | Calculated Ki | Drug ID | Calculated Ki |
| 1 | 1921 | 5.84E−13 | 2303 | 9.35E−15 | 1647 | 2.40E−10 | 1253 | 5.82E−14 | 2303 | 5.23E−10 |
| 2 | 2303 | 1.16E−12 | 2287 | 9.63E−12 | 1909 | 2.24E−07 | 560 | 6.07E−11 | 1921 | 9.39E−09 |
| 3 | 685 | 2.67E−12 | 893 | 1.34E−11 | 1584 | 4.49E−07 | 275 | 2.74E−10 | 1117 | 1.25E−08 |
| 4 | 1570 | 2.71E−12 | 1559 | 3.51E−11 | 1921 | 4.56E−07 | 1921 | 3.41E−10 | 2287 | 1.31E−08 |
| 5 | 1469 | 3.69E−12 | 685 | 3.67E−11 | 550 | 4.77E−07 | 472 | 4.71E−10 | 637 | 1.45E−08 |
| 6 | 1462 | 4.15E−12 | 1565 | 3.74E−11 | 2174 | 8.87E−07 | 551 | 5.94E−10 | 499 | 1.95E−08 |
| 7 | 1065 | 1.53E−11 | 1117 | 5.12E−11 | 2216 | 1.05E−06 | 274 | 7.46E−10 | 1462 | 2.48E−08 |
| 8 | 2262 | 2.45E−11 | 548 | 5.73E−11 | 2289 | 1.64E−06 | 2321 | 8.89E−10 | 1973 | 2.64E−08 |
| 9 | 893 | 4.79E−11 | 1921 | 7.02E−11 | 551 | 1.89E−06 | 2330 | 9.22E−10 | 2125 | 2.69E−08 |
| 10 | 2287 | 6.19E−11 | 492 | 7.72E−11 | 1721 | 1.89E−06 | 1777 | 9.58E−10 | 155 | 2.84E−08 |
| 11 | 1607 | 6.59E−11 | 1951 | 8.57E−11 | 818 | 1.99E−06 | 1647 | 1.00E−09 | 550 | 2.97E−08 |
| 12 | 1364 | 7.52E−11 | 1198 | 1.29E−10 | 1 | 2.26E−06 | 1920 | 1.21E−09 | 551 | 2.98E−08 |
| 13 | 1438 | 7.97E−11 | 2174 | 1.29E−10 | 1979 | 2.28E−06 | 1951 | 1.30E−09 | 2018 | 3.43E−08 |
| 14 | 546 | 8.84E−11 | 245 | 1.80E−10 | 36 | 2.34E−06 | 499 | 1.44E−09 | 492 | 3.76E−08 |
| 15 | 2174 | 1.46E−10 | 552 | 1.91E−10 | 1767 | 2.40E−06 | 2216 | 1.54E−09 | 548 | 3.81E−08 |
| 16 | 1565 | 1.62E−10 | 553 | 1.92E−10 | 1309 | 2.49E−06 | 545 | 1.94E−09 | 2216 | 4.03E−08 |
| 17 | 553 | 2.61E−10 | 919 | 2.06E−10 | 637 | 2.51E−06 | 1666 | 2.15E−09 | 1607 | 4.15E−08 |
| 18 | 670 | 2.70E−10 | 2328 | 3.25E−10 | 1839 | 2.96E−06 | 1680 | 2.44E−09 | 893 | 4.35E−08 |
| 19 | 637 | 3.00E−10 | 2300 | 3.41E−10 | 1968 | 3.01E−06 | 1257 | 2.54E−09 | 2322 | 5.03E−08 |
| 20 | 919 | 3.69E−10 | 482 | 3.73E−10 | 545 | 3.39E−06 | 2329 | 2.54E−09 | 553 | 5.33E−08 |
| 21 | 1117 | 4.02E−10 | 1680 | 3.75E−10 | 9 | 3.57E−06 | 205 | 3.12E−09 | 1951 | 5.98E−08 |
| 22 | 552 | 4.46E−10 | 2321 | 3.85E−10 | 533 | 3.60E−06 | 2289 | 3.55E−09 | 1680 | 6.14E−08 |
| 23 | 877 | 4.47E−10 | 1915 | 3.95E−10 | 1287 | 3.66E−06 | 479 | 3.92E−09 | 350 | 6.22E−08 |
| 24 | 1934 | 4.64E−10 | 1138 | 4.30E−10 | 553 | 3.76E−06 | 1569 | 4.04E−09 | 2321 | 6.41E−08 |
| 25 | 2330 | 6.41E−10 | 670 | 4.88E−10 | 822 | 3.88E−06 | 553 | 4.17E−09 | 439 | 7.10E−08 |
| 26 | 490 | 7.72E−10 | 554 | 5.22E−10 | 278 | 3.91E−06 | 2301 | 4.30E−09 | 2058 | 7.60E−08 |
| 27 | 2216 | 8.45E−10 | 489 | 6.16E−10 | 1437 | 4.01E−06 | 1641 | 4.38E−09 | 17 | 7.80E−08 |
| 28 | 1198 | 1.03E−09 | 2216 | 6.17E−10 | 552 | 4.04E−06 | 663 | 4.55E−09 | 274 | 7.90E−08 |
| 29 | 2295 | 1.33E−09 | 2074 | 7.39E−10 | 1716 | 4.42E−06 | 993 | 4.68E−09 | 670 | 8.01E−08 |
| 30 | 491 | 1.34E−09 | 1570 | 7.96E−10 | 334 | 4.45E−06 | 1973 | 4.80E−09 | 1985 | 8.24E−08 |
| 31 | 1556 | 1.42E−09 | 1805 | 8.10E−10 | 618 | 4.50E−06 | 236 | 4.82E−09 | 245 | 8.26E−08 |
| 32 | 399 | 1.44E−09 | 1989 | 8.44E−10 | 736 | 4.63E−06 | 2288 | 5.18E−09 | 603 | 8.38E−08 |
| 33 | 2009 | 2.06E−09 | 1065 | 8.59E−10 | 506 | 4.95E−06 | 494 | 5.43E−09 | 1989 | 9.26E−08 |
| 34 | 232 | 2.17E−09 | 603 | 8.62E−10 | 1939 | 5.19E−06 | 1176 | 5.60E−09 | 2301 | 1.01E−07 |
| 35 | 1951 | 2.28E−09 | 1462 | 9.04E−10 | 348 | 5.22E−06 | 893 | 5.69E−09 | 521 | 1.02E−07 |
| 36 | 2259 | 2.35E−09 | 490 | 9.06E−10 | 1783 | 5.24E−06 | 637 | 6.01E−09 | 501 | 1.08E−07 |
| 37 | 379 | 2.55E−09 | 1732 | 9.27E−10 | 548 | 5.26E−06 | 759 | 6.08E−09 | 2300 | 1.14E−07 |
| 38 | 1680 | 2.78E−09 | 2123 | 9.41E−10 | 1654 | 5.47E−06 | 711 | 6.20E−09 | 271 | 1.16E−07 |
| 39 | 482 | 2.85E−09 | 478 | 9.53E−10 | 488 | 5.55E−06 | 208 | 6.48E−09 | 2289 | 1.35E−07 |
| 40 | 2018 | 2.95E−09 | 505 | 9.84E−10 | 1634 | 5.57E−06 | 332 | 6.66E−09 | 2174 | 1.37E−07 |
| 41 | 488 | 2.97E−09 | 488 | 1.13E−09 | 1663 | 5.70E−06 | 164 | 6.74E−09 | 2134 | 1.41E−07 |
| 42 | 2177 | 3.07E−09 | 494 | 1.14E−09 | 567 | 5.89E−06 | 2171 | 7.14E−09 | 736 | 1.44E−07 |
| 43 | 2053 | 3.22E−09 | 546 | 1.16E−09 | 590 | 6.01E−06 | 492 | 7.20E−09 | 567 | 1.61E−07 |
| 44 | 548 | 3.52E−09 | 550 | 1.17E−09 | 1140 | 6.05E−06 | 1831 | 7.55E−09 | 545 | 1.62E−07 |
| 45 | 1323 | 3.53E−09 | 1973 | 1.33E−09 | 482 | 6.15E−06 | 1603 | 8.01E−09 | 554 | 1.63E−07 |
| 46 | 431 | 4.13E−09 | 545 | 1.36E−09 | 755 | 6.15E−06 | 488 | 8.22E−09 | 472 | 1.77E−07 |
| 47 | 1631 | 4.55E−09 | 127 | 1.37E−09 | 1780 | 6.38E−06 | 867 | 8.41E−09 | 574 | 1.85E−07 |
| 48 | 1860 | 4.79E−09 | 485 | 1.39E−09 | 1237 | 6.49E−06 | 2303 | 8.50E−09 | 482 | 1.87E−07 |
| 49 | 1246 | 4.91E−09 | 1438 | 1.42E−09 | 2155 | 6.69E−06 | 573 | 8.57E−09 | 1837 | 1.93E−07 |
| 50 | 1708 | 5.73E−09 | 2288 | 1.47E−09 | 1973 | 6.74E−06 | 448 | 9.20E−09 | 613 | 1.96E−07 |
| 51 | 1915 | 6.12E−09 | 1469 | 1.55E−09 | 621 | 7.07E−06 | 2308 | 9.20E−09 | 759 | 1.97E−07 |
| 52 | 1552 | 7.50E−09 | 637 | 1.57E−09 | 1960 | 7.12E−06 | 480 | 9.29E−09 | 552 | 2.00E−07 |
| 53 | 1972 | 7.57E−09 | 1521 | 1.62E−09 | 2078 | 7.31E−06 | 1834 | 9.30E−09 | 491 | 2.03E−07 |
| 54 | 502 | 7.96E−09 | 491 | 1.66E−09 | 563 | 7.42E−06 | 1 | 9.48E−09 | 2288 | 2.07E−07 |
| 55 | 1609 | 7.96E−09 | 2329 | 1.71E−09 | 546 | 7.55E−06 | 2125 | 9.49E−09 | 1672 | 2.17E−07 |
| 56 | 702 | 8.15E−09 | 232 | 1.78E−09 | 1985 | 7.64E−06 | 554 | 9.88E−09 | 546 | 2.25E−07 |
| 57 | 238 | 8.57E−09 | 493 | 1.82E−09 | 1525 | 7.70E−06 | 1585 | 9.90E−09 | 1938 | 2.49E−07 |
| 58 | 618 | 8.59E−09 | 271 | 1.83E−09 | 1807 | 7.71E−06 | 1708 | 1.07E−08 | 593 | 2.58E−07 |
| 59 | 496 | 9.08E−09 | 1622 | 2.00E−09 | 549 | 7.72E−06 | 1748 | 1.07E−08 | 586 | 2.61E−07 |
| 60 | 551 | 9.77E−09 | 481 | 2.03E−09 | 628 | 7.86E−06 | 1390 | 1.08E−08 | 164 | 2.68E−07 |
| 61 | 2289 | 1.02E−08 | 2239 | 2.14E−09 | 547 | 8.17E−06 | 2174 | 1.08E−08 | 1223 | 2.73E−07 |
| 62 | 904 | 1.10E−08 | 2289 | 2.17E−09 | 450 | 8.28E−06 | 1099 | 1.14E−08 | 1438 | 2.73E−07 |
| 63 | 495 | 1.16E−08 | 2151 | 2.23E−09 | 759 | 8.38E−06 | 1205 | 1.16E−08 | 822 | 2.75E−07 |
| 64 | 1658 | 1.16E−08 | 2343 | 2.31E−09 | 17 | 8.63E−06 | 552 | 1.19E−08 | 566 | 2.89E−07 |
| 65 | 2321 | 1.18E−08 | 318 | 2.37E−09 | 1357 | 8.73E−06 | 550 | 1.20E−08 | 36 | 3.02E−07 |
| 66 | 2288 | 1.21E−08 | 591 | 2.41E−09 | 1994 | 8.81E−06 | 845 | 1.20E−08 | 115 | 3.05E−07 |
| 67 | 1989 | 1.26E−08 | 2089 | 2.53E−09 | 1823 | 8.86E−06 | 486 | 1.22E−08 | 1967 | 3.12E−07 |
| 68 | 1866 | 1.28E−08 | 2134 | 2.66E−09 | 2309 | 9.16E−06 | 1321 | 1.22E−08 | 1418 | 3.16E−07 |
| 69 | 155 | 1.30E−08 | 711 | 3.20E−09 | 984 | 9.27E−06 | 2009 | 1.22E−08 | 939 | 3.21E−07 |
| 70 | 2171 | 1.38E−08 | 486 | 3.23E−09 | 1748 | 9.27E−06 | 1497 | 1.23E−08 | 2152 | 3.23E−07 |
| 71 | 1561 | 1.40E−08 | 1287 | 3.30E−09 | 1453 | 9.45E−06 | 271 | 1.26E−08 | 1774 | 3.25E−07 |
| 72 | 2215 | 1.42E−08 | 1968 | 3.35E−09 | 2195 | 9.62E−06 | 2239 | 1.27E−08 | 449 | 3.41E−07 |
| 73 | 873 | 1.64E−08 | 2215 | 3.35E−09 | 1067 | 9.80E−06 | 2215 | 1.30E−08 | 867 | 3.52E−07 |
| 74 | 27 | 1.65E−08 | 439 | 3.38E−09 | 566 | 9.85E−06 | 318 | 1.32E−08 | 544 | 3.57E−07 |
| 75 | 1433 | 1.73E−08 | 2232 | 3.46E−09 | 1969 | 1.01E−05 | 1583 | 1.37E−08 | 357 | 3.58E−07 |
| 76 | 939 | 1.80E−08 | 1934 | 3.68E−09 | 1611 | 1.02E−05 | 702 | 1.38E−08 | 1658 | 3.59E−07 |
| 77 | 617 | 1.84E−08 | 238 | 4.03E−09 | 2284 | 1.02E−05 | 1908 | 1.40E−08 | 818 | 3.60E−07 |
| 78 | 504 | 1.93E−08 | 1 | 4.18E−09 | 2039 | 1.07E−05 | 944 | 1.41E−08 | 1 | 3.76E−07 |
| 79 | 350 | 2.05E−08 | 1472 | 4.42E−09 | 310 | 1.10E−05 | 310 | 1.46E−08 | 429 | 3.78E−07 |
| 80 | 2221 | 2.06E−08 | 346 | 4.54E−09 | 641 | 1.10E−05 | 1634 | 1.49E−08 | 882 | 3.81E−07 |
| 81 | 332 | 2.15E−08 | 2125 | 4.57E−09 | 375 | 1.11E−05 | 873 | 1.52E−08 | 1437 | 3.84E−07 |
| 82 | 1620 | 2.18E−08 | 2018 | 4.68E−09 | 1961 | 1.12E−05 | 1086 | 1.53E−08 | 79 | 3.92E−07 |
| 83 | 1985 | 2.21E−08 | 604 | 4.69E−09 | 1938 | 1.13E−05 | 247 | 1.55E−08 | 1469 | 3.93E−07 |
| 84 | 481 | 2.23E−08 | 2336 | 4.79E−09 | 2134 | 1.14E−05 | 1108 | 1.55E−08 | 2257 | 4.00E−07 |
| 85 | 1973 | 2.25E−08 | 2155 | 4.86E−09 | 2287 | 1.15E−05 | 2154 | 1.55E−08 | 1274 | 4.15E−07 |
| 86 | 2028 | 2.25E−08 | 199 | 4.94E−09 | 152 | 1.16E−05 | 2155 | 1.58E−08 | 379 | 4.22E−07 |
| 87 | 205 | 2.29E−08 | 449 | 4.96E−09 | 1872 | 1.17E−05 | 2320 | 1.58E−08 | 1476 | 4.28E−07 |
| 88 | 2328 | 2.29E−08 | 1417 | 5.00E−09 | 1631 | 1.18E−05 | 436 | 1.60E−08 | 445 | 4.29E−07 |
| 89 | 346 | 2.44E−08 | 759 | 5.01E−09 | 1358 | 1.20E−05 | 503 | 1.62E−08 | 681 | 4.35E−07 |
| 90 | 857 | 2.46E−08 | 480 | 5.08E−09 | 77 | 1.23E−05 | 1418 | 1.62E−08 | 284 | 4.38E−07 |
| 91 | 1009 | 2.46E−08 | 2322 | 5.09E−09 | 1628 | 1.23E−05 | 2277 | 1.65E−08 | 1357 | 4.42E−07 |
| 92 | 1382 | 2.46E−08 | 1672 | 5.31E−09 | 436 | 1.25E−05 | 603 | 1.66E−08 | 502 | 4.48E−07 |
| 93 | 492 | 2.52E−08 | 1679 | 5.35E−09 | 2320 | 1.25E−05 | 192 | 1.67E−08 | 617 | 4.48E−07 |
| 94 | 36 | 2.63E−08 | 350 | 5.45E−09 | 639 | 1.34E−05 | 1175 | 1.67E−08 | 490 | 4.49E−07 |
| 95 | 545 | 2.79E−08 | 2156 | 5.48E−09 | 374 | 1.36E−05 | 1306 | 1.68E−08 | 495 | 4.55E−07 |
| 96 | 73 | 2.81E−08 | 567 | 5.55E−09 | 480 | 1.39E−05 | 502 | 1.70E−08 | 592 | 4.64E−07 |
| 97 | 1040 | 2.93E−08 | 484 | 5.59E−09 | 593 | 1.39E−05 | 506 | 1.70E−08 | 393 | 4.70E−07 |
| 98 | 246 | 3.00E−08 | 155 | 5.79E−09 | 1274 | 1.41E−05 | 1521 | 1.71E−08 | 1939 | 4.75E−07 |
| 99 | 992 | 3.06E−08 | 1917 | 5.87E−09 | 1593 | 1.41E−05 | 1395 | 1.80E−08 | 2343 | 4.81E−07 |
| [symbol] 00 | 2125 | 3.09E−08 | 544 | 6.04E−09 | 993 | 1.42E−05 | 1607 | 1.82E−08 | 536 | 4.83E−07 |

| | | | |
|---|---|---|---|
| 1RQ2 | 1UZR | 1ZAU | 2C27 |

| [symbol] ank | Drug ID | Calculated Ki | Drug ID | Calculated Ki | Drug ID | Calculated Ki | Drug ID | Calculated Ki |
|---|---|---|---|---|---|---|---|---|
| 1 | 2303 | 1.62E−09 | 551 | 4.81E−08 | 893 | 1.11E−10 | 274 | 2.18E−10 |
| 2 | 2321 | 1.67E−09 | 586 | 3.90E−07 | 637 | 2.20E−10 | 492 | 7.97E−10 |
| 3 | 2216 | 3.58E−09 | 553 | 4.09E−07 | 1921 | 2.55E−10 | 2216 | 9.04E−10 |
| 4 | 2330 | 4.41E−09 | 550 | 4.69E−07 | 919 | 3.22E−10 | 1585 | 1.49E−09 |
| 5 | 1921 | 5.82E−09 | 546 | 5.52E−07 | 2174 | 3.43E−10 | 2321 | 2.11E−09 |
| 6 | 232 | 6.51E−09 | 547 | 5.68E−07 | 482 | 3.51E−10 | 1256 | 2.21E−09 |
| 7 | 759 | 6.90E−09 | 1899 | 8.01E−07 | 1951 | 4.35E−10 | 2289 | 2.82E−09 |
| 8 | 2287 | 7.71E−09 | 637 | 8.19E−07 | 1805 | 5.87E−10 | 845 | 2.99E−09 |
| 9 | 1445 | 9.31E−09 | 593 | 8.42E−07 | 1565 | 6.53E−10 | 1834 | 3.16E−09 |
| 10 | 2288 | 9.92E−09 | 758 | 8.71E−07 | 490 | 6.57E−10 | 505 | 3.33E−09 |
| 11 | 637 | 1.03E−08 | 521 | 9.79E−07 | 818 | 7.15E−10 | 236 | 3.48E−09 |
| 12 | 893 | 1.04E−08 | 1866 | 1.04E−06 | 2303 | 8.24E−10 | 1710 | 3.79E−09 |
| 13 | 1585 | 1.10E−08 | 1631 | 1.08E−06 | 552 | 1.29E−09 | 448 | 4.66E−09 |
| 14 | 702 | 1.25E−08 | 2089 | 1.21E−06 | 566 | 1.45E−09 | 2303 | 5.21E−09 |
| 15 | 155 | 1.34E−08 | 1921 | 1.22E−06 | 2216 | 2.41E−09 | 1250 | 5.51E−09 |
| 16 | 1934 | 1.52E−08 | 642 | 1.27E−06 | 488 | 2.63E−09 | 880 | 5.52E−09 |
| 17 | 552 | 1.54E−08 | 552 | 1.33E−06 | 1469 | 3.03E−09 | 436 | 5.85E−09 |
| 18 | 472 | 1.62E−08 | 517 | 1.41E−06 | 36 | 3.78E−09 | 2288 | 6.00E−09 |
| 19 | 2162 | 1.63E−08 | 1570 | 1.42E−06 | 553 | 3.88E−09 | 1257 | 6.26E−09 |
| 20 | 1721 | 1.70E−08 | 386 | 1.47E−06 | 1462 | 3.96E−09 | 1089 | 6.32E−09 |
| 21 | 2009 | 1.72E−08 | 643 | 1.48E−06 | 2089 | 4.06E−09 | 2172 | 6.76E−09 |
| 22 | 449 | 1.78E−08 | 1968 | 1.52E−06 | 759 | 4.35E−09 | 1908 | 6.92E−09 |
| 23 | 553 | 1.86E−08 | 591 | 1.56E−06 | 499 | 4.69E−09 | 346 | 7.33E−09 |
| 24 | 164 | 2.45E−08 | 1332 | 1.61E−06 | 2289 | 5.98E−09 | 545 | 7.78E−09 |
| 25 | 462 | 2.51E−08 | 1860 | 1.79E−06 | 603 | 6.18E−09 | 2055 | 7.85E−09 |
| 26 | 1641 | 2.53E−08 | 561 | 1.84E−06 | 485 | 6.83E−09 | 552 | 8.62E−09 |
| 27 | 486 | 2.59E−08 | 1566 | 1.93E−06 | 479 | 7.56E−09 | 577 | 8.99E−09 |
| 28 | 681 | 2.76E−08 | 393 | 1.99E−06 | 481 | 7.84E−09 | 486 | 9.06E−09 |
| 29 | 1512 | 2.79E−08 | 544 | 2.03E−06 | 2232 | 8.38E−09 | 310 | 9.09E−09 |
| 30 | 2322 | 2.79E−08 | 282 | 2.10E−06 | 484 | 8.40E−09 | 1641 | 9.59E−09 |
| 31 | 548 | 3.38E−08 | 1607 | 2.11E−06 | 2134 | 8.54E−09 | 2023 | 9.63E−09 |
| 32 | 489 | 3.41E−08 | 434 | 2.12E−06 | 546 | 8.61E−09 | 462 | 1.01E−08 |
| 33 | 436 | 3.50E−08 | 17 | 2.19E−06 | 586 | 9.47E−09 | 2174 | 1.02E−08 |
| 34 | 550 | 3.58E−08 | 163 | 2.23E−06 | 1 | 9.76E−09 | 2287 | 1.03E−08 |
| 35 | 274 | 3.65E−08 | 237 | 2.24E−06 | 551 | 1.15E−08 | 491 | 1.08E−08 |
| 36 | 445 | 3.68E−08 | 618 | 2.28E−06 | 685 | 1.19E−08 | 2330 | 1.12E−08 |
| 37 | 1 | 3.83E−08 | 893 | 2.38E−06 | 567 | 1.28E−08 | 1154 | 1.16E−08 |
| 38 | 71 | 3.95E−08 | 507 | 2.43E−06 | 2156 | 1.29E−08 | 1420 | 1.16E−08 |
| 39 | 499 | 3.95E−08 | 270 | 2.44E−06 | 314 | 1.31E−08 | 1973 | 1.22E−08 |
| 40 | 478 | 4.45E−08 | 1033 | 2.46E−06 | 1548 | 1.49E−08 | 1450 | 1.40E−08 |
| 41 | 551 | 4.55E−08 | 1837 | 2.56E−06 | 1973 | 1.51E−08 | 1248 | 1.57E−08 |
| 42 | 2289 | 4.57E−08 | 36 | 2.62E−06 | 476 | 1.64E−08 | 889 | 1.59E−08 |
| 43 | 1679 | 4.95E−08 | 1523 | 2.68E−06 | 483 | 1.65E−08 | 893 | 1.63E−08 |
| 44 | 545 | 5.25E−08 | 556 | 2.73E−06 | 495 | 1.65E−08 | 450 | 1.67E−08 |
| 45 | 1274 | 5.25E−08 | 906 | 2.74E−06 | 2034 | 1.65E−08 | 1274 | 1.68E−08 |
| 46 | 448 | 5.64E−08 | 1815 | 2.82E−06 | 127 | 1.67E−08 | 1086 | 1.76E−08 |
| 47 | 1666 | 6.35E−08 | 628 | 2.94E−06 | 1585 | 1.68E−08 | 2232 | 1.82E−08 |
| 48 | 993 | 6.52E−08 | 616 | 2.99E−06 | 2287 | 1.71E−08 | 823 | 1.83E−08 |
| 49 | 823 | 6.73E−08 | 1322 | 3.02E−06 | 548 | 1.72E−08 | 1205 | 1.84E−08 |
| 50 | 546 | 7.02E−08 | 936 | 3.05E−06 | 71 | 1.75E−08 | 1680 | 1.89E−08 |
| 51 | 944 | 7.02E−08 | 596 | 3.12E−06 | 1089 | 1.78E−08 | 216 | 1.90E−08 |
| 52 | 2301 | 7.26E−08 | 612 | 3.13E−06 | 2125 | 1.79E−08 | 472 | 1.90E−08 |
| 53 | 904 | 7.27E−08 | 529 | 3.21E−06 | 1570 | 1.82E−08 | 546 | 1.91E−08 |
| 54 | 1672 | 7.40E−08 | 1683 | 3.22E−06 | 2330 | 1.82E−08 | 975 | 1.92E−08 |
| 55 | 882 | 7.57E−08 | 1431 | 3.25E−06 | 1710 | 1.91E−08 | 1622 | 1.93E−08 |
| 56 | 1622 | 7.86E−08 | 571 | 3.27E−06 | 521 | 1.99E−08 | 473 | 2.04E−08 |
| 57 | 115 | 7.87E−08 | 896 | 3.30E−06 | 1767 | 2.06E−08 | 553 | 2.19E−08 |
| 58 | 1099 | 7.90E−08 | 1492 | 3.32E−06 | 1438 | 2.07E−08 | 2308 | 2.21E−08 |
| 59 | 605 | 8.07E−08 | 1017 | 3.42E−06 | 274 | 2.16E−08 | 1603 | 2.22E−08 |
| 60 | 1944 | 8.25E−08 | 1295 | 3.46E−06 | 2009 | 2.17E−08 | 1355 | 2.26E−08 |
| 61 | 1785 | 8.95E−08 | 1117 | 3.49E−06 | 1569 | 2.29E−08 | 702 | 2.31E−08 |
| 62 | 1985 | 8.98E−08 | 450 | 3.52E−06 | 593 | 2.33E−08 | 1652 | 2.33E−08 |
| 63 | 593 | 9.01E−08 | 875 | 3.62E−06 | 1205 | 2.35E−08 | 488 | 2.34E−08 |
| 64 | 2262 | 9.12E−08 | 1821 | 3.64E−06 | 478 | 2.40E−08 | 538 | 2.41E−08 |
| 65 | 1260 | 9.32E−08 | 1 | 3.72E−06 | 2300 | 2.51E−08 | 890 | 2.46E−08 |
| 66 | 554 | 9.40E−08 | 615 | 3.77E−06 | 492 | 2.55E−08 | 499 | 2.55E−08 |
| 67 | 27 | 9.68E−08 | 3 | 3.78E−06 | 1596 | 2.55E−08 | 1748 | 2.57E−08 |
| 68 | 350 | 9.68E−08 | 2065 | 3.82E−06 | 2155 | 2.59E−08 | 550 | 2.59E−08 |
| 69 | 1576 | 9.86E−08 | 1919 | 3.84E−06 | 2288 | 2.61E−08 | 1708 | 2.59E−08 |
| 70 | 460 | 9.89E−08 | 617 | 3.92E−06 | 545 | 2.65E−08 | 502 | 2.64E−08 |
| 71 | 1845 | 9.94E−08 | 871 | 3.93E−06 | 2018 | 2.71E−08 | 1076 | 2.66E−08 |
| 72 | 467 | 9.98E−08 | 427 | 3.94E−06 | 350 | 2.82E−08 | 449 | 2.79E−08 |
| 73 | 1634 | 1.00E−07 | 1009 | 3.94E−06 | 449 | 2.90E−08 | 818 | 3.11E−08 |
| 74 | 1631 | 1.01E−07 | 1611 | 3.94E−06 | 550 | 2.90E−08 | 484 | 3.22E−08 |
| 75 | 1973 | 1.05E−07 | 657 | 3.97E−06 | 2309 | 2.92E−08 | 1607 | 3.25E−08 |
| 76 | 661 | 1.08E−07 | 1985 | 3.97E−06 | 480 | 2.94E−08 | 1287 | 3.26E−08 |
| 77 | 2320 | 1.19E−07 | 971 | 3.99E−06 | 1562 | 2.97E−08 | 759 | 3.29E−08 |
| 78 | 1176 | 1.21E−07 | 502 | 4.00E−06 | 1274 | 3.06E−08 | 1108 | 3.44E−08 |
| 79 | 1301 | 1.24E−07 | 312 | 4.01E−06 | 2138 | 3.13E−08 | 711 | 3.46E−08 |
| 80 | 1947 | 1.26E−07 | 357 | 4.04E−06 | 1631 | 3.14E−08 | 554 | 3.47E−08 |
| 81 | 272 | 1.30E−07 | 534 | 4.07E−06 | 1722 | 3.25E−08 | 27 | 3.54E−08 |
| 82 | 2125 | 1.30E−07 | 1806 | 4.13E−06 | 487 | 3.29E−08 | 348 | 3.55E−08 |
| 83 | 346 | 1.31E−07 | 916 | 4.15E−06 | 1249 | 3.36E−08 | 127 | 3.56E−08 |
| 84 | 760 | 1.31E−07 | 545 | 4.19E−06 | 334 | 3.37E−08 | 2322 | 3.66E−08 |
| 85 | 577 | 1.34E−07 | 1973 | 4.19E−06 | 1908 | 3.39E−08 | 1418 | 3.76E−08 |
| 86 | 1117 | 1.34E−07 | 1291 | 4.20E−06 | 2213 | 3.40E−08 | 506 | 3.84E−08 |
| 87 | 877 | 1.36E−07 | 1917 | 4.21E−06 | 882 | 3.48E−08 | 567 | 3.86E−08 |
| 88 | 1154 | 1.36E−07 | 457 | 4.33E−06 | 1129 | 3.51E−08 | 637 | 3.96E−08 |
| 89 | 1548 | 1.36E−07 | 1534 | 4.33E−06 | 436 | 3.54E−08 | 800 | 4.02E−08 |
| 90 | 339 | 1.39E−07 | 505 | 4.37E−06 | 1780 | 3.55E−08 | 131 | 4.03E−08 |
| 91 | 1075 | 1.44E−07 | 155 | 4.42E−06 | 1108 | 3.57E−08 | 478 | 4.10E−08 |
| 92 | 1910 | 1.45E−07 | 647 | 4.54E−06 | 1559 | 3.61E−08 | 1306 | 4.13E−08 |
| 93 | 1570 | 1.47E−07 | 1931 | 4.54E−06 | 1985 | 3.70E−08 | 566 | 4.27E−08 |
| 94 | 1951 | 1.48E−07 | 1868 | 4.68E−06 | 506 | 3.73E−08 | 1634 | 4.27E−08 |
| 95 | 601 | 1.52E−07 | 939 | 4.75E−06 | 463 | 3.94E−08 | 1 | 4.36E−08 |
| 96 | 2342 | 1.53E−07 | 1359 | 4.82E−06 | 1497 | 3.94E−08 | 482 | 4.38E−08 |
| 97 | 1569 | 1.54E−07 | 1912 | 4.88E−06 | 822 | 4.03E−08 | 551 | 4.42E−08 |
| 98 | 2274 | 1.55E−07 | 1111 | 4.89E−06 | 1666 | 4.07E−08 | 1629 | 4.47E−08 |
| 99 | 501 | 1.56E−07 | 603 | 4.91E−06 | 163 | 4.09E−08 | 496 | 4.62E−08 |
| 100 | 477 | 1.61E−07 | 31 | 5.00E−06 | 877 | 4.21E−08 | 2134 | 4.66E−08 |

[symbol]

indicates data missing or illegible when filed

### REFERENCES

1. Hammer, S. M., et al. JAMA 296: 827-843, 2006
2. Volberding, P. A., et al. (1995) A comparison of immediate with deferred zidovudine therapy for asymptomatic HIV-infected adults with CD4 cell counts of 500 or more per cubic millimeter. AIDS Clinical Trials Group. N Engl J Med 333, 401-407
3. Rusconi, S., et al. (1996) Viral load, viral phenotype modification, zidovudine susceptibility and reverse transcriptase mutations during the first 6 months of zidovudine monotherapy in HIV-1-infected people. Antivir Ther 1, 211-219
4. Metzner, K. J., et al. (2007) Rapid selection of drug-resistant HIV-1 during the first months of suppressive ART in treatment-naive patients. Aids 21, 703-711
5. Jones, R., and Gazzard, B. (2006) The cost of antiretroviral drugs and influence on prescribing policies. Int J STD AIDS 17, 499-506
6. Bisson, G. P., et al. (2006) Out-of-pocket costs of HAART limit HIV treatment responses in Botswana's private sector. Aids 20, 1333-1336
7. Moya, J., et al. (2006) Limitations of a simplification antiretroviral strategy for HIV-infected patients with decreasing adherence to a protease inhibitor regimen. HIV Clin Trials 7, 210-214
8. Faivre, S., et al. (2006) Current development of mTOR inhibitors as anticancer agents. Nat Rev Drug Discov 5, 671-688
9. Millan, M. J. (2004) The role of monoamines in the actions of established and “novel” antidepressant agents: a critical review. Eur J Pharmacol 500, 371-384
10. Wong, M. L., et al. (2003) Identification, characterization, and gene expression profiling of endotoxin-induced myocarditis. Proc Natl Acad Sci USA 100, 14241-14246
11. Celotti, F., and Laufer, S. (2001) Anti-inflammatory drugs: new multitarget compounds to face an old problem. The dual inhibition concept. Pharmacol Res 43, 429-436
12. Bugatti, A., et al. (2007) Heparin-mimicking sulfonic acid polymers as multitarget inhibitors of HIV-1 Tat and gp 120 proteins. Antimicrob Agents Chemother
13. Litovchick, A., et al. (2001) Neomycin B-arginine conjugate, a novel HIV-1 Tat antagonist: synthesis and anti-HIV activities. Biochemistry 40, 15612-15623
14. Santhosh, K. C., et al. (2001) Correlation of anti-HIV activity with anion spacing in a series of cosalane analogues with extended polycarboxylate pharmacophores. J Med Chem 44, 703-714
15. Kuchimanchi, K. R., et al. (2001) Binding of cosalane—a novel highly lipophilic anti-HIV agent—to albumin and glycoprotein. J Pharm Sci 90, 659-666
16. De Clercq, E. (2001) New developments in anti-HIV chemotherapy. Curr Med Chem 8, 1543-1572
17. Casimiro-Garcia, A., et al. (2000) Synthesis and anti-HIV activity of cosalane analogues incorporating nitrogen in the linker chain. Bioorg Med Chem 8, 191-200
18. Cushman, M., et al. (1995) Cosalane analogues with enhanced potencies as inhibitors of HIV-1 protease and integrase. J Med Chem 38, 443-452
19. Cushman, M., et al. (1994) Design, synthesis, and biological evaluation of cosalane, a novel anti-HIV agent which inhibits multiple features of virus reproduction. J Med Chem 37, 3040-3050
20. Shoichet, B. K. (2004) Virtual screening of chemical libraries. Nature 432, 862-865
21. Jenkins, J. L., et al. (2003) Virtual screening to enrich hit lists from high-throughput screening: a case study on small-molecule inhibitors of angiogenin. Proteins 50, 81-93
22. Mestres, J. (2002) Virtual screening: a real screening complement to high-throughput screening. Biochem Soc Trans 30, 797-799
23. Kovacs, J. A., and Masur, H. (2000) Prophylaxis against opportunistic infections in patients with human immunodeficiency virus infection. N Engl J Med 342, 1416-1429
24. Pitter, C., et al. (2007) Cost-effectiveness of cotrimoxazole prophylaxis among persons with HIV in Uganda. J Acquir Immune Defic Syndr 44, 336-343
25. Desai, P. V., et al. (2006) Identification of novel parasitic cysteine protease inhibitors by use of virtual screening. 2. The available chemical directory. J Med Chem 49, 1576-1584
26. Desai, P. V., et al. (2004) Identification of novel parasitic cysteine protease inhibitors using virtual screening. 1. The ChemBridge database. J Med Chem 47, 6609-6615
27. Chong, C. R., et al. (2006) A clinical drug library screen identifies astemizole as an antimalarial agent. Nat Chem Biol 2, 415-416
28. Weisman, J. L., et al. (2006) Searching for new antimalarial therapeutics amongst known drugs. Chem Biol Drug Des 67, 409-416
29. Jenwitheesuk, E., and Samudrala, R. (2005) Identification of potential multitarget antimalarial drugs. JAMA 294, 1490-1491
30. Jenwitheesuk, E., and Samudrala, R. (2003) Improved prediction of HIV-1 protease-inhibitor binding energies by molecular dynamics simulations. BMC Struct Biol 3, 2
31. Jenwitheesuk, E., and Samudrala, R. (2005) Prediction of HIV-1 protease inhibitor resistance using a protein-inhibitor flexible docking approach. Antivir Ther 10, 157-166
32. Barratt, M. D. (2000) Prediction of toxicity from chemical structure. Cell Biol Toxicol 16, 1-13
33. Benigni, R., and Zito, R. (2003) Designing safer drugs: (Q)SAR-based identification of mutagens and carcinogens. Curr Top Med Chem 3, 1289-1300
34. Dearden, J. C. (2003) In silico prediction of drug toxicity. J Comput Aided Mol Des 17, 119-127
35. Greene, N. (2002) Computer systems for the prediction of toxicity: an update. Adv Drug Deliv Rev 54, 417-431
36. Roche, O., and Guba, W. (2005) Computational chemistry as an integral component of lead generation. Mini Rev Med Chem 5, 677-683
37. Swamidass, S. J., et al. (2005) Kernels for small molecules and the prediction of mutagenicity, toxicity and anti-cancer activity. Bioinformatics 21 Suppl 1, i359-368
38. Zmuidinavicius, D., et al. (2003) Progress in toxinformatics: the challenge of predicting acute toxicity. Curr Top Med Chem 3, 1301-1314
39. Wexler, P. (1995) TOXNET: the National Library of Medicine's toxicology database. Am Fam Physician 52, 1677-1678
40. Li, A. P. (2001) Screening for human ADME/Tox drug properties in drug discovery. Drug Discov Today 6, 357-366
41. Vidal, D., et al. (2006) A novel search engine for virtual screening of very large databases. J Chem Inf Model 46, 836-843
42. Pereira, F. B., et al. (1997) Membrane fusion induced by the HIV type 1 fusion peptide: modulation by factors affecting glycoprotein 41 activity and potential anti-HIV compounds. AIDS Res Hum Retroviruses 13, 1203-1211
43. Oki, T., et al. (1990) Pradimicins A, B and C: new antifungal antibiotics. II. In vitro and in vivo biological activities. J Antibiot (Tokyo) 43, 763-770
44. Marco, F., et al. (1998) In vitro activities of voriconazole (UK-109,496) and four other antifungal agents against 394 clinical isolates of Candida spp. Antimicrob Agents Chemother 42, 161-163
45. Vazquez, J. A., et al. (1997) In vitro activity of a new pneumocandin antifungal, L-743,872, against azole-susceptible and -resistant Candida species. Antimicrob Agents Chemother 41, 1612-1614
46. Tzatzarakis, M. N., et al. (2001) Comparison of in vitro activities of amphotericin, clotrimazole, econazole, miconazole, and nystatin against Fusarium oxysporum. J Environ Sci Health B 36, 331-340
47. Kessler, H. A., et al. (1981) Effects of amphotericin B on hepatitis B virus. Antimicrob Agents Chemother 20, 826-833
48. Kohler, S., et al. (2000) Comparison of the echinocandin caspofungin with amphotericin B for treatment of histoplasmosis following pulmonary challenge in a murine model. Antimicrob Agents Chemother 44, 1850-1854
49. Fesen, M. R., et al. (1993) Inhibitors of human immunodeficiency virus integrase. Proc Natl Acad Sci USA 90, 2399-2403
50. Levitz, S. M., et al. (1997) Chloroquine induces human mononuclear phagocytes to inhibit and kill Cryptococcus neoformans by a mechanism independent of iron deprivation. J Clin Invest 100, 1640-1646
51. Tsai, W. P., et al. (1990) Inhibition of human immunodeficiency virus infectivity by chloroquine. AIDS Res Hum Retroviruses 6, 481-489
52. Crowle, A. J., and May, M. H. (1990) Inhibition of tubercle bacilli in cultured human macrophages by chloroquine used alone and in combination with streptomycin, isoniazid, pyrazinamide, and two metabolites of vitamin D3. Antimicrob Agents Chemother 34, 2217-2222
53. Jiang, M. C., et al. (1996) Inhibition of HIV-1 Tat-mediated transactivation by quinacrine and chloroquine. Biochem. Biophys Res Commun 226, 1-7
54. Ang, K. K., et al. (2000) In vivo antimalarial activity of the beta-carboline alkaloid manzamine A. Antimicrob Agents Chemother 44, 1645-1649
55. Fivelman, Q. L., et al. (1999) The effect of artesunate combined with standard antimalarials against chloroquine-sensitive and chloroquine-resistant strains of Plasmodium falciparum in vitro. Trans R Soc Trop Med Hyg 93, 429-432
56. Artico, M., et al. (1998) Geometrically and conformationally restrained cinnamoyl compounds as inhibitors of HIV-1 integrase: synthesis, biological evaluation, and molecular modeling. J Med Chem 41, 3948-3960
57. Yang, F., et al. (2005) Curcumin inhibits formation of amyloid beta oligomers and fibrils, binds plaques, and reduces amyloid in vivo. J Biol Chem 280, 5892-5901
58. Hooker, C. W., et al. (2001) Inhibitors of human immunodeficiency virus type 1 reverse transcriptase target distinct phases of early reverse transcription. J Virol 75, 3095-3104
59. Li, M., et al. (2007) Curcumin, a dietary component, has anticancer, chemosensitization, and radiosensitization effects by down-regulating the MDM2 oncogene through the PI3K/mTOR/ETS2 pathway. Cancer Res 67, 1988-1996
60. Srivastava, K. C., et al. (1995) Curcumin, a major component of food spice turmeric (Curcuma longa) inhibits aggregation and alters eicosanoid metabolism in human blood platelets. Prostaglandins Leukot Essent Fatty Acids 52, 223-227
61. Braaten, D., et al. (1996) Cyclosporine A-resistant human immunodeficiency virus type 1 mutants demonstrate that Gag encodes the functional target of cyclophilin A. J Virol 70, 5170-5176
62. Onyewu, C., et al. (2003) Ergosterol biosynthesis inhibitors become fungicidal when combined with calcineurin inhibitors against Candida albicans, Candida glabrata, and Candida krusei. Antimicrob Agents Chemother 47, 956-964
63. Odom, A., et al. (1997) The immunosuppressant FK506 and its nonimmunosuppressive analog L-685,818 are toxic to Cryptococcus neoformans by inhibition of a common target protein. Antimicrob Agents Chemother 41, 156-161
64. Perkins, M. E., et al. (1998) Cyclosporin analogs inhibit in vitro growth of Cryptosporidium parvum. Antimicrob Agents Chemother 42, 843-848
65. Nakagawa, M., et al. (2004) Specific inhibition of hepatitis C virus replication by cyclosporin A. Biochem Biophys Res Commun 313, 42-47
66. Silverman, J. A., et al. (1997) Characterization of anti-Toxoplasma activity of SDZ 215-918, a cyclosporin derivative lacking immunosuppressive and peptidyl-prolyl-isomerase-inhibiting activity: possible role of a P glycoprotein in Toxoplasma physiology. Antimicrob Agents Chemother 41, 1859-1866
67. Damaso, C. R., and Moussatche, N. (1998) Inhibition of vaccinia virus replication by cyclosporin A analogues correlates with their affinity for cellular cyclophilins. J Gen Virol 79 (Pt 2), 339-346
68. Jayasuriya, H., et al. (2002) Durhamycin A, a potent inhibitor of HIV Tat transactivation. J Nat Prod 65, 1091-1095
69. Gordon, M. A., and Lapa, E. W. (1966) Durhamycin, a Pentaene Antifungal Antibiotic from Streptomyces durhamensis sp. n. Appl Microbiol 14, 754-760
70. http://dtp.nci.nih.gov (2007)
71. Nikolaeva, L., and Galabov, A. S. (2000) Antiviral effect of the combination of enviroxime and disoxaril on coxsackievirus B1 infection. Acta Virol 44, 73-78
72. Ninomiya, Y., et al. (1990) Comparative studies on the antirhinovirus activity and the mode of action of the rhinovirus capsid binding agents, chalcone amides. Antiviral Res 13, 61-74
73. Nikolaeva, L., and Galabov, A. S. (1995) Synergistic inhibitory effect of enviroxime and disoxaril on poliovirus type 1 replication. Acta Virol 39, 235-241
74. Watanabe, N., et al. (2006) Fumagillin suppresses HIV-1 infection of macrophages through the inhibition of Vpr activity. FEBS Lett 580, 2598-2602
75. Franssen, F. F., et al. (1995) Susceptibility of Encephalitozoon cuniculi to several drugs in vitro. Antimicrob Agents Chemother 39, 1265-1268
76. Didier, E. S. (1997) Effects of albendazole, fumagillin, and TNP-470 on microsporidial replication in vitro. Antimicrob Agents Chemother 41, 1541-1546
77. Molina, J. M., et al. (1997) Potential efficacy of fumagillin in intestinal microsporidiosis due to Enterocytozoon bieneusi in patients with HIV infection: results of a drug screening study. The French Microsporidiosis Study Group. Aids 11, 1603-1610
78. Zhang, P., et al. (2002) Angiogenesis inhibitors specific for methionine aminopeptidase 2 as drugs for malaria and leishmaniasis. J Biomed Sci 9, 34-40
79. Warhurst, D. C., et al. (2003) Hydroxychloroquine is much less active than chloroquine against chloroquine-resistant Plasmodium falciparum, in agreement with its physicochemical properties. J Antimicrob Chemother 52, 188-193
80. Sperber, K., et al. (1993) Inhibition of human immunodeficiency virus type 1 replication by hydroxychloroquine in T cells and monocytes. AIDS Res Hum Retroviruses 9, 91-98
81. Mimoto, T., et al. (1999) Structure-activity relationship of small-sized HIV protease inhibitors containing allophenylnorstatine. J Med Chem 42, 1789-1802
82. Clemente, J. C., et al. (2006) Structure of the aspartic protease plasmepsin 4 from the malarial parasite Plasmodium malariae bound to an allophenylnorstatine-based inhibitor. Acta Crystallogr D Biol Crystallogr 62, 246-252
83. Wondrak, E. M., et al. (1988) Inhibition of HIV-1 RNA-dependent DNA polymerase and cellular DNA polymerases alpha, beta and gamma by phosphonoformic acid and other drugs. J Antimicrob Chemother 21, 151-161
84. Giacometti, A., et al. (1996) In-vitro activity of macrolides alone and in combination with artemisin, atovaquone, dapsone, minocycline or pyrimethamine against Cryptosporidium parvum. J Antimicrob Chemother 38, 399-408
85. Lemaitre, M., et al. (1990) Protective activity of tetracycline analogs against the cytopathic effect of the human immunodeficiency viruses in CEM cells. Res Virol 141, 5-16
86. Boucher, H. W., et al. (2000) In vitro activities of the glycylcycline GAR-936 against gram-positive bacteria. Antimicrob Agents Chemother 44, 2225-2229
87. Wallace, R. J., Jr., et al. (2002) Comparison of the in vitro activity of the glycylcycline tigecycline (formerly GAR-936) with those of tetracycline, minocycline, and doxycycline against isolates of nontuberculous mycobacteria. Antimicrob Agents Chemother 46, 3164-3167
88. Collins, L., and Franzblau, S. G. (1997) Microplate alamar blue assay versus BACTEC 460 system for high-throughput screening of compounds against Mycobacterium tuberculosis and Mycobacterium avium. Antimicrob Agents Chemother 41, 1004-1009
89. Arai, S., et al. (1992) Antimycoplasmal activities of new quinolones, tetracyclines, and macrolides against Mycoplasma pneumoniae. Antimicrob Agents Chemother 36, 1322-1324
90. Chang, H. R., et al. (1991) Activity of minocycline against Toxoplasma gondii infection in mice. J Antimicrob Chemother 27, 639-645
91. Yahi, N., et al. (1994) Suramin inhibits binding of the V3 region of HIV-1 envelope glycoprotein gp120 to galactosylceramide, the receptor for HIV-1 gp120 on human colon epithelial cells. J Biol Chem 269, 24349-24353
92. Baba, M., et al. (1993) Selective inhibition of human cytomegalovirus replication by naphthalenedisulfonic acid derivatives. Antiviral Res 20, 223-233
93. Tewtrakul, S., et al. (2003) HIV-1 integrase inhibitory substances from Coleus parvifolius. Phytother Res 17, 232-239
94. Aguilar, J. S., et al. (1999) The polysulfonated compound suramin blocks adsorption and lateral diffusion of herpes simplex virus type-1 in vero cells. Virology 258, 141-151
95. Debyser, Z., et al. (1991) An antiviral target on reverse transcriptase of human immunodeficiency virus type 1 revealed by tetrahydroimidazo-[4,5,1-jk] [1,4]benzodiazepin-2 (1H)-one and -thione derivatives. Proc Natl Acad Sci USA 88, 1451-1455
96. Hui, E. K., and Nayak, D. P. (2002) Role of G protein and protein kinase signalling in influenza virus budding in MDCK cells. J Gen Virol 83, 3055-3066
97. Crance, J. M., et al. (1997) Inhibition of sandfly fever Sicilian virus (Phlebovirus) replication in vitro by antiviral compounds. Res Virol 148, 353-365

Each recited range includes all combinations and sub-combinations of ranges, as well as specific numerals contained therein.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating herpesvirus infection in a mammalian subject comprising administering to the mammalian subject a pharmaceutical composition in an amount effective to reduce or eliminate infection by two or more classes or species of herpesvirus or to prevent its occurrence or recurrence in the mammalian subject.

2. The method of claim 1 wherein the class of herpesvirus is α-herpesvirus, β-herpesvirus, or γ-herpesvirus.

3. The method of claim 1 wherein the species of herpesvirus is herpes simplex virus, cytomegalovirus, Kaposi's sarcoma virus, varicella zoster virus, or Epstein Barr virus.

4. The method of claim 1 wherein the composition is an inhibitor of a herpesvirus protease.

5. The method of claim 3 wherein the composition comprises meso-5,10,15,20-Tetrakis-(N-methyl-4-pyridyl)porphine tetratosylate (TMPyP4).

6. The method of claim 4 further comprising administering the herpesvirus protease inhibitor in combination with a nucleoside analog.

7. The method of claim 6 wherein the herpesvirus protease inhibitor is TMPyP4 and the nucleoside analog is acyclovir.

8. A method for treating Plasmodium falciparum infection in a mammalian subject comprising administering to the mammalian subject a pharmaceutical composition capable of inhibiting two or more Plasmodium falciparum target proteins, in an amount effective to reduce or eliminate the Plasmodium falciparum infection or to prevent its occurrence or recurrence in the mammalian subject.

9. The method of claim 8 wherein the pharmaceutical composition is KN62 (ID 274).

10. The method of claim 8 wherein the pharmaceutical composition is u-74389g (ID 2321).

11. The method of claim 8 wherein the pharmaceutical composition is daunorubicin (ID 1989).

12. The method of claim 8 wherein the pharmaceutical composition is nitrotetrazolium bt (ID 2174).

13. The method of claim 8 wherein the pharmaceutical composition is STI-571/Imatinib (ID 637).

14. The method of claim 8 wherein the pharmaceutical composition is TMPyP4 (ID 2303).

15. The method of claim 8 wherein the pharmaceutical composition is telomerase inhibitor v (ID 2288), bisindolylmaleimide iii (ID 546), methylgene_{—}05 (ID 463), remiszewski_{—}013 (ID 449), remiszewski_{—}010 (ID 448), phthalylsulfathiazole (ID 1576), or sulfaphenazole (ID 916).

16. A method for treating human immunodeficiency virus infection in a mammalian subject comprising administering to the mammalian subject a pharmaceutical composition comprising an inhibitor of HIV integrase in an amount effective to reduce or eliminate infection by human immunodeficiency virus or to prevent its occurrence or recurrence in the mammalian subject.

17. The method of claim 16, wherein the HIV integrase inhibitor is TMPyP4, calmidazolium chloride, paromomycin, aurintricarboxylic acid, ro 31-8220 (548), dichlorobenzamil (36), catenulin (1198), kanamycin (670), or capreomycin (893).

18. A method for treating microbial infection in a mammalian subject comprising administering to the mammalian subject a pharmaceutical composition comprising meso-5,10,15,20-Tetrakis-(N-methyl-4-pyridyl)porphine tetratosylate (TMPyP4) in an amount effective to reduce or eliminate the microbial infection or to prevent its occurrence or recurrence in the mammalian subject.

19. The method of claim 18 wherein the microbial infection is a viral infection, bacterial infection, or parasitic infection.

20. The method claim 19 wherein the microbial infection is herpesvirus, human immunodeficiency virus, or Plasmodium falciparum.

21. A method for identifying a candidate peptide inhibitor or candidate peptidomimetic inhibitor of a protein target for treatment of disease comprising:
performing a stability analysis using a protein structure energy function to identify highly stable, partially surface-exposed elements of the protein target,
designing peptide inhibitors or peptidomimetic inhibitors having the same amino acid sequence as the highly stable elements or having amino acid sequences that interacts with the highly stable element,
designing derivative inhibitors by computationally mutating side chains of the peptide inhibitors or peptidomimetic inhibitors and evaluating the protein structure energy of the derivative inhibitors, and
identifying the derivative inhibitor having a lower protein structure energy than the peptide inhibitors or peptidomimetic inhibitors, wherein the derivative inhibitor is the candidate peptide inhibitor or peptidomimetic inhibitor of the protein target for treatment of disease.

22. The method of claim 21 further comprising identifying derivative inhibitors as candidate peptide inhibitors or candidate peptidomimetic inhibitors of two or more highly stable elements in one protein target.

23. The method of claim 22 wherein the candidate peptide inhibitors or candidate peptidomimetic inhibitors target one or more diseases.

24. The method of claim 21 further comprising identifying the candidate peptide inhibitor or the candidate peptidomimetic inhibitor of homologous highly stable elements in two or more protein targets.

25. The method of claim 24 wherein the candidate peptide inhibitor or the candidate peptidomimetic inhibitor target one or more diseases.

26. The method of claim 21, wherein the highly stable element is a secondary structure element, a tertiary structure element, or a quaternary structure element.

27. The method of claim 21 wherein the disease is bacterial disease, viral disease, parasitic disease, or neoplastic disease.

28. A computer readable medium bearing computer executable instructions for carrying out the method of claim 21.

29. A modulated data signal carrying computer executable instructions for performing the method of claim 21.

30. At least one computing device comprising means for performing the method of claim 21.

31. A method for predicting inhibitors of two or more protein targets for treatment of one or more diseases which comprises:
providing a set of experimentally-synthesized or naturally-occurring compounds,
calculating a binding affinity for each compound against a multiplicity of protein targets, and
ranking each compound by inhibitory concentration based upon calculation of binding affinity against each of the one or more protein targets for treatment of disease.

32. The method of claim 31 wherein the set of experimental or naturally-occurring compounds are approved by the U.S. Food and Drug Administration.

33. The method of claim 31 wherein the set of experimentally-synthesized or naturally-occurring compounds has been screened for one or more of toxicity, absorption, distribution, metabolism excretion, or pharmacokinetics.

34. The method of claim 31, further comprising calculating the binding affinity using a docking with dynamics protocol.

35. The method of claim 31 further comprising ranking each compound by inhibitory concentration against two or more protein targets for treatment of disease.

36. The method of claim 31 further comprising ranking each compound by inhibitory concentration against the protein target for treatment of two or more diseases.

37. The method of claim 31 wherein the disease is bacterial disease, viral disease, parasitic disease, or neoplastic disease.

38. The method of claim 31 further comprising predicting the inhibitor for treatment of disease by calculating the highest binding affinity.

39. A computer readable medium bearing computer executable instructions for carrying out the method of claim 31.

40. A modulated data signal carrying computer executable instructions for performing the method of claim 31.

41. At least one computing device comprising means for performing the method of claim 31.

42. A method for predicting inhibitors of one or more protein targets for treatment of one or more diseases which comprises:
providing a set of experimentally-synthesized or naturally-occurring compounds,
clustering the compounds by structural similarity,
calculating a binding affinity for one or more compounds representing each structurally similar cluster against one or more protein targets,
ranking each representative compound by inhibitory concentration based upon calculation of binding affinity against each of the one or more protein targets for the disease or the disease-causing organism,
selecting one or more high-ranking clusters of compounds,
ranking compounds within the one or more high-ranking clusters based upon calculation of binding affinity against each of the one or more protein targets for the disease, and
predicting high-ranking compounds as inhibitors of one or more protein target for treatment of the one or more diseases.

43. The method of claim 42 wherein the set of experimentally-synthesized or naturally-occurring compounds are approved by the U.S. Food and Drug Administration.

44. The method of claim 42 wherein the set of experimentally-synthesized or naturally-occurring compounds has been screened for one or more of toxicity, absorption, distribution, metabolism excretion, or pharmacokinetics.

45. The method of claim 42 further comprising calculating the binding affinity using a docking with dynamics protocol.

46. The method of claim 42 further comprising ranking each compound by inhibitory concentration against two or more protein targets for treatment of the disease.

47. The method of claim 42 further comprising ranking each compound by inhibitory concentration against the protein target for treatment of two or more diseases.

48. The method of claim 42 wherein the disease is bacterial disease, viral disease, parasitic disease, or neoplastic disease.

49. The method of claim 42 further comprising predicting the inhibitor for treatment of disease using the lowest inhibitory concentration to calculate the highest binding affinity.

50. The method of claim 42 further comprising reducing the screening time to predict inhibitors of one or more protein target for treatment of disease.

51. A computer readable medium bearing computer executable instructions for carrying out the method of claim 42.

52. A modulated data signal carrying computer executable instructions for performing the method of claim 42.

53. At least one computing device comprising means for performing the method of claim 42.

54. A method for predicting inhibitors of two or more protein targets for treatment of one or more disease in a mammalian subject which comprises:
providing a set of experimentally-synthesized or naturally-occurring compounds,
calculating a binding affinity using a docking with dynamics protocol for one or more compounds against two or more protein targets,
ranking compounds by inhibitory concentration based upon calculation of binding affinity against each of the two or more protein targets for the disease or a disease-causing organism, and
predicting high-ranking compounds as inhibitors of two or more protein target for treatment of the one or more diseases.

55. The method of claim 54 wherein the set of experimentally-synthesized or naturally-occurring compounds are approved by the U.S. Food and Drug Administration.

56. The method of claim 54 wherein the set of experimentally-synthesized or naturally-occurring compounds has been screened for one or more of toxicity, absorption, distribution, metabolism excretion, or pharmacokinetics.

57. The method of claim 54 wherein the disease is bacterial disease, viral disease, parasitic disease, or neoplastic disease.

58. The method of claim 54 further comprising predicting the inhibitor for treatment of disease using the lowest inhibitory concentration to calculate the highest binding affinity.

59. A computer readable medium bearing computer executable instructions for carrying out the method of claim 54.

60. A modulated data signal carrying computer executable instructions for performing the method of claim 54.

61. At least one computing device comprising means for performing the method of claim 54.
